SEQUENCE LISTING (124 sequences)

SEQ ID NO: 1 - DNA, 255 bp, Nostoc punctiforme
atgagccaaacggaactttttgaaaaggtcaagaaaatcgtcatcgaacaactgagtgttgaagatgcttccaaaatcactccacaagctaagtttatggaagatttaggagctgattccctggatactgttgaactcgtgatggctttggaagaagaatttgatatcgaaattcccgacgaagctgccgagcagattgtatcggttcaagacgcagtagattacatcaataacaaagttgctgcatcagcttaa

SEQ ID NO: 2 - protein, 84 aa, Nostoc punctiforme
MSQTELFEKVKKIVIEQLSVEDASKITPQAKFMEDLGADSLDTVELVMALEEEFDIEIPDEAAEQIVSVQDAVDYINNKVAASA

SEQ ID NO: 3 - DNA, 234 bp, Synechocystis sp.
atgaatcaggaaatttttgaaaaagtaaaaaaaatcgtcgtggaacagttggaagtggatcctgacaaagtgacccccgatgccacctttgccgaagatttaggggctgattccctcgatacagtggaattggtcatggccctggaagaagagtttgatattgaaattcccgatgaagtggcggaaaccattgataccgtgggcaaagccgttgagcatatcgaaagtaaataa

SEQ ID NO: 4 - protein, 77 aa, Synechocystis sp.
MNQEIFEKVKKIVVEQLEVDPDKVTPDATFAEDLGADSLDTVELVMALEEEFDIEIPDEVAETIDTVGKAVEHIESK

SEQ ID NO: 5 - DNA, 243 bp, Prochlorococcus marinus
atgtcacaagaagaaatccttcaaaaagtatgctctattgtttctgagcaactaagtgttgaatcagccgaagtaaaatctgattcaaactttcaaaatgatttaggtgcagactccctagacaccgtagagctagttatggctcttgaagaagcatttgatatcgagatacctgatgaagcagctgaaggtatcgcaacagtaggagatgctgttaaattcatcgaagaaaaaaaaggttaa

SEQ ID NO: 6 - protein, 80 aa, Prochlorococcus marinus
MSQEEILQKVCSIVSEQLSVESAEVKSDSNFQNDLGADSLDTVELVMALEEAFDIEIPDEAAEGIATVGDAVKFIEEKKG

SEQ ID NO: 7 - DNA, 243 bp, Synechococcus elongatus
atgagccaagaagacatcttcagcaaagtcaaagacattgtggctgagcagctgagtgtggatgtggctgaagtcaagccagaatccagcttccaaaacgatctgggagcggactcgctggacaccgtggaactggtgatggctctggaagaggctttcgatatcgaaatccccgatgaagccgctgaaggcattgcgaccgttcaagacgccgtcgatttcatcgctagcaaagctgcctag

SEQ ID NO: 8 - protein, 80 aa, Synechococcus elongatus
MSQEDIFSKVKDIVAEQLSVDVAEVKPESSFQNDLGADSLDTVELVMALEEAFDIEIPDEAAEGIATVQDAVDFIASKAA

SEQ ID NO: 9 - DNA, 255 bp, Nostoc sp.
atgagccaatcagaaacttttgaaaaagtcaaaaaaattgttatcgaacaactaagtgtggagaaccctgacacagtaactccagaagctagttttgccaacgatttacaggctgattccctcgatacagtagaactagtaatggctttggaagaagaatttgatatcgaaattcccgatgaagccgcagagaaaattaccactgttcaagaagcggtggattacatcaataaccaagttgccgcatcagcttaa

SEQ ID NO: 10 - protein, 84 aa, Nostoc sp.
MSQSETFEKVKKIVIEQLSVENPDTVTPEASFANDLQADSLDTVELVMALEEEFDIEIPDEAAEKITTVQEAVDYINNQVAASA

SEQ ID NO: 11 - DNA, 674 bp, Artificial Sequence
  source: /note=“Description of Artificial Sequence Synthetic polynucleotide”
atgaagatttacggaatttatatggaccgcccgctttcacaggaagaaaatgaacggttcatgactttcatatcacctgaaaaacgggagaaatgccggagattttatcataaagaagatgctcaccgcaccctgctgggagatgtgctcgttcgctcagtcataagcaggcagtatcagttggacaaatccgatatccgctttagcacgcaggaatacgggaagccgtgcatccctgatcttcccgacgctcatttcaacatttctcactccggccgctgggtcattggtgcgtttgattcacagccgatcggcatagatatcgaaaaaacgaaaccgatcagccttgagatcgccaagcgcttcttttcaaaaacagagtacagcgaccttttagcaaaagacaaggacgagcagacagactatttttatcatctatggtcaatgaaagaaagctttatcaaacaggaaggcaaaggcttatcgcttccgcttgattccttttcagtgcgcctgcatcaggacggacaagtatccattgagcttccggacagccattccccatgctatatcaaaacgtatgaggtcgatcccggctacaaaatggctgtatgcgccgcacaccctgtttccccgaggatatcacaatggtctcgtacgaagagcttttataa

SEQ ID NO: 12 - protein, 224 aa, Artificial Sequence
  source: /note=“Description of Artificial Sequence Synthetic polypeptide”
MKIYGIYMDRPLSQEENERFMTFISPEKREKCRRFYHKEDAHRTLLGDVLVRSVISRQYQLDKSDIRFSTQEYGKPCIPDLPDAHFNISHSGRWVIGAFDSQPIGIDIEKTKPISLEIAKRFFSKTEYSDLLAKDKDEQTDYFYHLWSMKESFIKQEGKGLSLPLDSFSVRLHQDGQVSIELPDSHSPCYIKTYEVDPGYKMAVCAAHPDFPEDITMVSYEELL

SEQ ID NO: 13 - DNA, 52 bp, Artificial Sequence
  source: /note=“Description of Artificial Sequence Synthetic primer”
ggcaatttgagaatttaaggaggaaaacaaaatgagccaagaagacatcttc

SEQ ID NO: 14 - DNA, 33 bp, Artificial Sequence
  source: /note=“Description of Artificial Sequence Synthetic primer”
cccaagcttcgaattcctaggcagctttgctag

SEQ ID NO: 15 - DNA, 56 bp, Artificial Sequence
  source: /note=“Description of Artificial Sequence Synthetic primer”
ggcaatttgagaatttaaggaggaaaacaaaatgaatcaggaaatttttgaaaaag

SEQ ID NO: 16 - DNA, 41 bp, Artificial Sequence
  source: /note=“Description of Artificial Sequence Synthetic primer”
cccaagcttcgaattcttatttactttcgatatgctcaacg

SEQ ID NO: 17 - DNA, 53 bp, Artificial Sequence
  source: /note=“Description of Artificial Sequence Synthetic primer”
ggcaatttgagaatttaaggaggaaaacaaaatgtcacaagaagaaatccttc

SEQ ID NO: 18 - DNA, 46 bp, Artificial Sequence
  source: /note=“Description of Artificial Sequence Synthetic primer”
cccaagcttcgaattcttaaccttttttttcttcgatgaatttaac

SEQ ID NO: 19 - DNA, 53 bp, Artificial Sequence
  source: /note=“Description of Artificial Sequence Synthetic primer”
ggcaatttgagaatttaaggaggaaaacaaaatgagccaaacggaactttttg

SEQ ID NO: 20 - DNA, 38 bp, Artificial Sequence
  source: /note=“Description of Artificial Sequence Synthetic primer”
cccaagcttcgaattcttaagctgatgcagcaactttg

SEQ ID NO: 21 - DNA, 53 bp, Artificial Sequence
  source: /note=“Description of Artificial Sequence Synthetic primer”
ggcaatttgagaatttaaggaggaaaacaaaatgagccaatcagaaacttttg

SEQ ID NO: 22 - DNA, 34 bp, Artificial Sequence
  source: /note=“Description of Artificial Sequence Synthetic primer”
cccaagcttcgaattcttaagctgatgcggcaac

SEQ ID NO: 23 - DNA, 43 bp, Artificial Sequence
  source: /note=“Description of Artificial Sequence Synthetic primer”
agctgcctaggaatttaaggaggaataaaccatgaagatttac

SEQ ID NO: 24 - DNA, 43 bp, Artificial Sequence
  source: /note=“Description of Artificial Sequence Synthetic primer”
aaaaggttaagaatttaaggaggaataaaccatgaagatttac

SEQ ID NO: 25 - DNA, 43 bp, Artificial Sequence
  source: /note=“Description of Artificial Sequence Synthetic primer”
atcagcttaagaatttaaggaggaataaaccatgaagatttac

SEQ ID NO: 26 - DNA, 41 bp, Artificial Sequence
  source: /note=“Description of Artificial Sequence Synthetic primer”
cccaagcttcgaattcttataaaagctcttcgtacgagacc

SEQ ID NO: 27 - DNA, 69 bp, Artificial Sequence
  source: /note=“Description of Artificial Sequence Synthetic primer”
gaaatcacgcatctgcgtttgcaataataatagaggaggataactaaatgagtacagttgaagagcgcg

SEQ ID NO: 28 - DNA, 45 bp, Artificial Sequence
  source: /note=“Description of Artificial Sequence Synthetic primer”
gccaagctggagaccgtttaaactcaggtgtgcgcgacaatgtag

SEQ ID NO: 29 - DNA, 23 bp, Artificial Sequence
  source: /note=“Description of Artificial Sequence Synthetic primer”
taatagaggaggataactaaatg

SEQ ID NO: 30 - DNA, 27 bp, Artificial Sequence
  source: /note=“Description of Artificial Sequence Synthetic primer”
ttattgcaaacgcagatgcgtgatttc

SEQ ID NO: 31 - DNA, 23 bp, Artificial Sequence
  source: /note=“Description of Artificial Sequence Synthetic primer”
gtttaaacggtctccagcttggc

SEQ ID NO: 32 - DNA, 69 bp, Artificial Sequence
  source: /note=“Description of Artificial Sequence Synthetic primer”
gaaatcacgcatctgcgtttgcaataataatagaggaggataactaaatgagcactatcgaagaacgcg

SEQ ID NO: 33 - DNA, 45 bp, Artificial Sequence
  source: /note=“Description of Artificial Sequence Synthetic primer”
gccaagctggagaccgtttaaacttacgcctggtggccgttgatg

SEQ ID NO: 34 - DNA, 696 bp, Synechococcus elongatus
atgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 35 - protein, 231 aa, Synechococcus elongatus
MPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 36 - DNA, 696 bp, Synechocystis sp.
atgcccgagcttgctgtccgcaccgaatttgactattccagcgaaatttacaaagacgcctatagccgcatcaacgccattgtcattgaaggcgaacaggaagcctacagcaactacctccagatggcggaactcttgccggaagacaaagaagagttgacccgcttggccaaaatggaaaaccgccataaaaaaggtttccaagcctgtggcaacaacctccaagtgaaccctgatatgccctatgcccaggaatttttcgccggtctccatggcaatttccagcacgcttttagcgaagggaaagttgttacctgtttattgatccaggctttgattatcgaagcttttgcgatcgccgcctataacatatatatccctgtggcggacgactttgctcggaaaatcactgagggcgtagtcaaggacgaatacacccacctcaactacggggaagaatggctaaaggccaactttgccaccgctaaggaagaactggagcaggccaacaaagaaaacctacccttagtgtggaaaatgctcaaccaagtgcagggggacgccaaggtattgggcatggaaaaagaagccctagtggaagattttatgatcagctacggcgaagccctcagtaacatcggcttcagcaccagggaaattatgcgtatgtcttcctacggtttggccggagtctag

SEQ ID NO: 37 - protein, 231 aa, Synechocystis sp.
MPELAVRTEFDYSSEIYKDAYSRINAIVIEGEQEAYSNYLQMAELLPEDKEELTRLAKMENRHKKGFQACGNNLQVNPDMPYAQEFFAGLHGNFQHAFSEGKVVTCLLIQALIIEAFAIAAYNIYIPVADDFARKITEGVVKDEYTHLNYGEEWLKANFATAKEELEQANKENLPLVWKMLNQVQGDAKVLGMEKEALVEDFMISYGEALSNIGFSTREIMRMSSYGLAGV

SEQ ID NO: 38 - DNA, 699 bp, Nostoc punctiforme
atgcagcagcttacagaccaatctaaagaattagatttcaagagcgaaacatacaaagatgcttatagccggattaatgcgatcgtgattgaaggggaacaagaagcccatgaaaattacatcacactagcccaactgctgccagaatctcatgatgaattgattcgcctatccaagatggaaagccgccataagaaaggatttgaagcttgtgggcgcaatttagctgttaccccagatttgcaatttgccaaagagtttttctccggcctacaccaaaattttcaaacagctgccgcagaagggaaagtggttacttgtctgttgattcagtctttaattattgaatgttttgcgatcgcagcatataacatttacatccccgttgccgacgatttcgcccgtaaaattactgaaggagtagttaaagaagaatacagccacctcaattttggagaagtttggttgaaagaacactttgcagaatccaaagctgaacttgaacttgcaaatcgccagaacctacccatcgtctggaaaatgctcaaccaagtagaaggtgatgcccacacaatggcaatggaaaaagatgctttggtagaagacttcatgattcagtatggtgaagcattgagtaacattggtttttcgactcgcgatattatgcgcttgtcagcctacggactcataggtgcttaa

SEQ ID NO: 39 - protein, 232 aa, Nostoc punctiforme
MQQLTDQSKELDFKSETYKDAYSRINAIVIEGEQEAHENYITLAQLLPESHDELIRLSKMESRHKKGFEACGRNLAVTPDLQFAKEFFSGLHQNFQTAAAEGKVVTCLLIQSLIIECFAIAAYNIYIPVADDFARKITEGVVKEEYSHLNFGEVWLKEHFAESKAELELANRQNLPIVWKMLNQVEGDAHTMAMEKDALVEDFMIQYGEALSNIGFSTRDIMRLSAYGLIGA

SEQ ID NO: 40 - DNA, 696 bp, Nostoc sp.
atgcagcaggttgcagccgatttagaaattgatttcaagagcgaaaaatataaagatgcctatagtcgcataaatgcgatcgtgattgaaggggaacaagaagcatacgagaattacattcaactatcccaactgctgccagacgataaagaagacctaattcgcctctcgaaaatggaaagccgtcacaaaaaaggatttgaagcttgtggacggaacctacaagtatcaccagatatggagtttgccaaagaattctttgctggactacacggtaacttccaaaaagcggcggctgaaggtaaaatcgttacctgtctattgattcagtccctgattattgaatgttttgcgatcgccgcatacaatatctacattcccgttgctgacgattttgctcgtaaaatcactgagggtgtagtcaaagatgaatacagccacctcaacttcggcgaagtttggttacagaaaaattttgcccaatccaaagcagaattagaagaagctaatcgtcataatcttcccatagtttggaaaatgctcaatcaagtcgcggatgatgccgcagtcttagctatggaaaaagaagccctagtcgaagattttatgattcagtacggcgaagcgttaagtaatattggcttcacaaccagagatattatgcggatgtcagcctacggacttacagcagcttaa

SEQ ID NO: 41 - protein, 231 aa, Nostoc sp.
MQQVAADLEIDFKSEKYKDAYSRINAIVIEGEQEAYENYIQLSQLLPDDKEDLIRLSKMESRHKKGFEACGRNLQVSPDMEFAKEFFAGLHGNFQKAAAEGKIVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 42 - DNA, 696 bp, Acaryochloris marina
atgccccaaactcaggctatttcagaaattgacttctatagtgacacctacaaagatgcttacagtcgtattgacggcattgtgatcgaaggtgagcaagaagcgcatgaaaactatattcgtcttggcgaaatgctgcctgagcaccaagacgactttatccgcctgtccaagatggaagcccgtcataagaaagggtttgaagcctgcggtcgcaacttaaaagtaacctgcgatctagactttgcccggcgtttcttttccgacttacacaagaattttcaagatgctgcagctgaggataaagtgccaacttgcttagtgattcagtccttgatcattgagtgttttgcgatcgcagcttacaacatctatatccccgtcgctgatgactttgcccgtaagattacagagtctgtggttaaggatgagtatcaacacctcaattatggtgaagagtggcttaaagctcacttcgatgatgtgaaagcagaaatccaagaagctaatcgcaaaaacctccccatcgtttggagaatgctgaacgaagtggacaaggatgcggccgttttaggaatggaaaaagaagccctggttgaagacttcatgatccagtatggtgaagcccttagcaatattggtttctctacaggcgaaattatgcggatgtctgcctatggtcttgtggctgcgtaa

SEQ ID NO: 43 - protein, 231 aa, Acaryochloris marina
MPQTQAISEIDFYSDTYKDAYSRIDGIVIEGEQEAHENYIRLGEMLPEHQDDFIRLSKMEARHKKGFEACGRNLKVTCDLDFARRFFSDLHKNFQDAAAEDKVPTCLVIQSLIIECFAIAAYNIYIPVADDFARKITESVVKDEYQHLNYGEEWLKAHFDDVKAEIQEANRKNLPIVWRMLNEVDKDAAVLGMEKEALVEDFMIQYGEALSNIGFSTGEIMRMSAYGLVAA

SEQ ID NO: 44 - DNA, 696 bp, Thermosynechococcus elongatus
atgacaacggctaccgctacacctgttttggactaccatagcgatcgctacaaggatgcctacagccgcattaacgccattgtcattgaaggtgaacaggaagctcacgataactatatcgatttagccaagctgctgccacaacaccaagaggaactcacccgccttgccaagatggaagctcgccacaaaaaggggtttgaggcctgtggtcgcaacctgagcgtaacgccagatatggaatttgccaaagccttctttgaaaaactgcgcgctaactttcagagggctctggcggagggaaaaactgcgacttgtcttctgattcaagctttgatcatcgaatcctttgcgatcgcggcctacaacatctacatcccaatggcggatcctttcgcccgtaaaattactgagagtgttgttaaggacgaatacagccacctcaactttggcgaaatctggctcaaggaacactttgaaagcgtcaaaggagagctcgaagaagccaatcgcgccaatttacccttggtctggaaaatgctcaaccaagtggaagcagatgccaaagtgctcggcatggaaaaagatgcccttgtggaagacttcatgattcagtacagtggtgccctagaaaatatcggctttaccacccgcgaaattatgaagatgtcagtttatggcctcactggggcataa

SEQ ID NO: 45 - protein, 231 aa, Thermosynechococcus elongatus
MTTATATPVLDYHSDRYKDAYSRINAIVIEGEQEAHDNYIDLAKLLPQHQEELTRLAKMEARHKKGFEACGRNLSVTPDMEFAKAFFEKLRANFQRALAEGKTATCLLIQALIIESFAIAAYNIYIPMADPFARKITESVVKDEYSHLNFGEIWLKEHFESVKGELEEANRANLPLVWKMLNQVEADAKVLGMEKDALVEDFMIQYSGALENIGFTTREIMKMSVYGLTGA

SEQ ID NO: 46 - DNA, 732 bp, Synechococcus sp.
atggccccagcgaacgtcctgcccaacacccccccgtcccccactgatgggggcggcactgccctagactacagcagcccaaggtatcggcaggcctactcccgcatcaacggtattgttatcgaaggcgaacaagaagcccacgacaactacctcaagctggccgaaatgctgccggaagctgcagaggagctgcgcaagctggccaagatggaattgcgccacatgaaaggcttccaggcctgcggcaaaaacctgcaggtggaacccgatgtggagtttgcccgcgcctttttcgcgcccttgcgggacaatttccaaagcgccgcagcggcaggggatctggtctcctgttttgtcattcagtctttgatcatcgagtgctttgccattgccgcctacaacatctacatcccggttgccgatgactttgcccgcaagatcaccgagggggtagttaaggacgagtatctgcacctcaattttggggagcgctggctgggcgagcactttgccgaggttaaagcccagatcgaagcagccaacgcccaaaatctgcctctagttcggcagatgctgcagcaggtagaggcggatgtggaagccatttacatggatcgcgaggccattgtagaagacttcatgatcgcctacggcgaggccctggccagcatcggcttcaacacccgcgaggtaatgcgcctctcggcccagggtctgcgggccgcctga

SEQ ID NO: 47 - protein, 243 aa, Synechococcus sp.
MAPANVLPNTPPSPTDGGGTALDYSSPRYRQAYSRINGIVIEGEQEAHDNYLKLAEMLPEAAEELRKLAKMELRHMKGFQACGKNLQVEPDVEFARAFFAPLRDNFQSAAAAGDLVSCFVIQSLIIECFAIAAYNIYIPVADDFARKITEGVVKDEYLHLNFGERWLGEHFAEVKAQIEAANAQNLPLVRQMLQQVEADVEAIYMDREAIVEDFMIAYGEALASIGFNTREVMRLSAQGLRAA

SEQ ID NO: 48 - DNA, 708 bp, Gloeobacter violaceus
gtgaaccgaaccgcaccgtccagcgccgcgcttgattaccgctccgacacctaccgcgatgcgtactcccgcatcaatgccatcgtccttgaaggcgagcgggaagcccacgccaactaccttaccctcgctgagatgctgccggaccatgccgaggcgctcaaaaaactggccgcgatggaaaatcgccacttcaaaggcttccagtcctgcgcccgcaacctcgaagtcacgccggacgacccgtttgcaagggcctacttcgaacagctcgacggcaactttcagcaggcggcggcagaaggtgaccttaccacctgcatggtcatccaggcactgatcatcgagtgcttcgcaattgcggcctacaacgtctacattccggtggccgacgcgtttgcccgcaaggtgaccgagggcgtcgtcaaggacgagtacacccacctcaactttgggcagcagtggctcaaagagcgcttcgtgaccgtgcgcgagggcatcgagcgcgccaacgcccagaatctgcccatcgtctggcggatgctcaacgccgtcgaagcggacaccgaagtgctgcagatggataaagaagcgatcgtcgaagactttatgatcgcctacggtgaagccttgggcgacatcggtttttcgatgcgcgacgtgatgaagatgtccgcccgcggccttgcctctgccccccgccagtga

SEQ ID NO: 49 - protein, 235 aa, Gloeobacter violaceus
MNRTAPSSAALDYRSDTYRDAYSRINAIVLEGEREAHANYLTLAEMLPDHAEALKKLAAMENRHFKGFQSCARNLEVTPDDPFARAYFEQLDGNFQQAAAEGDLTTCMVIQALIIECFAIAAYNVYIPVADAFARKVTEGVVKDEYTHLNFGQQWLKERFVTVREGIERANAQNLPIVWRMLNAVEADTEVLQMDKEAIVEDFMIAYGEALGDIGFSMRDVMKMSARGLASAPRQ

SEQ ID NO: 50 - DNA, 732 bp, Prochlorococcus marinus
atgcctacgcttgagatgcctgtggcagctgttcttgacagcactgttggatcttcagaagccctgccagacttcacttcagatagatataaggatgcatacagcagaatcaacgcaatagtcattgagggcgaacaggaagcccatgacaattacatcgcgattggcacgctgcttcccgatcatgtcgaagagctcaagcggcttgccaagatggagatgaggcacaagaagggctttacagcttgcggcaagaaccttggcgttgaggctgacatggacttcgcaagggagttttttgctcctttgcgtgacaacttccagacagctttagggcaggggaaaacacctacatgcttgctgatccaggcgctcttgattgaagcctttgctatttcggcttatcacacctatatccctgtttctgacccctttgctcgcaagattactgaaggtgtcgtgaaggacgagtacacacacctcaattatggcgaggcttggctcaaggccaatctggagagttgccgtgaggagttgcttgaggccaatcgcgagaacctgcctctgattcgccggatgcttgatcaggtagcaggtgatgctgccgtgctgcagatggataaggaagatctgattgaggatttcttaatcgcctaccaggaatctctcactgagattggctttaacactcgtgaaattacccgtatggcagcggcagctcttgtgagctga

SEQ ID NO: 51 - protein, 243 aa, Prochlorococcus marinus
MPTLEMPVAAVLDSTVGSSEALPDFTSDRYKDAYSRINAIVIEGEQEAHDNYIAIGTLLPDHVEELKRLAKMEMRHKKGFTACGKNLGVEADMDFAREFFAPLRDNFQTALGQGKTPTCLLIQALLIEAFAISAYHTYIPVSDPFARKITEGVVKDEYTHLNYGEAWLKANLESCREELLEANRENLPLIRRMLDQVAGDAAVLQMDKEDLIEDFLIAYQESLTEIGFNTREITRMAAAALVS

SEQ ID NO: 52 - DNA, 717 bp, Prochlorococcus marinus
atgcaaacactcgaatctaataaaaaaactaatctagaaaattctattgatttacccgattttactactgattcttacaaagacgcttatagcaggataaatgcaatagttattgaaggtgaacaagaggctcatgataattacatttccttagcaacattaattcctaacgaattagaagagttaactaaattagcgaaaatggagcttaagcacaaaagaggctttactgcatgtggaagaaatctaggtgttcaagctgacatgatttttgctaaagaattcttttccaaattacatggtaattttcaggttgcgttatctaatggcaagacaactacatgcctattaatacaggcaattttaattgaagcttttgctatatccgcgtatcacgtttacataagagttgctgatcctttcgcgaaaaaaattacccaaggtgttgttaaagatgaatatcttcatttaaattatggacaagaatggctaaaagaaaatttagcgacttgtaaagatgagctaatggaagcaaataaggttaaccttccattaatcaagaagatgttagatcaagtctcggaagatgcttcagtactagctatggatagggaagaattaatggaagaattcatgattgcctatcaggacactctccttgaaataggtttagataatagagaaattgcaagaatggcaatggctgctatagtttaa

SEQ ID NO: 53 - protein, 238 aa, Prochlorococcus marinus
MQTLESNKKTNLENSIDLPDFTTDSYKDAYSRINAIVIEGEQEAHDNYISLATLIPNELEELTKLAKMELKHKRGFTACGRNLGVQADMIFAKEFFSKLHGNFQVALSNGKTTTCLLIQAILIEAFAISAYHVYIRVADPFAKKITQGVVKDEYLHLNYGQEWLKENLATCKDELMEANKVNLPLIKKMLDQVSEDASVLAMDREELMEEFMIAYQDTLLEIGLDNREIARMAMAAIV

SEQ ID NO: 54 - DNA, 726 bp, Prochlorococcus marinus
atgcaagcttttgcatccaacaatttaaccgtagaaaaagaagagctaagttctaactctcttccagatttcacctcagaatcttacaaagatgcttacagcagaatcaatgcagttgtaattgaaggggagcaagaagcttattctaattttcttgatctcgctaaattgattcctgaacatgcagatgagcttgtgaggctagggaagatggagaaaaagcatatgaatggtttttgtgcttgcgggagaaatcttgctgtaaagcctgatatgccttttgcaaagacctttttctcaaaactccataataattttttagaggctttcaaagtaggagatacgactacctgtctcctaattcaatgcatcttgattgaatcttttgcaatatccgcatatcacgtttatatacgtgttgctgatccattcgccaaaagaatcacagagggtgttgtccaagatgaatacttgcatttgaactatggtcaagaatggcttaaggccaatctagagacagttaagaaagatcttatgagggctaataaggaaaacttgcctcttataaagtccatgctcgatgaagtttcaaacgacgccgaagtccttcatatggataaagaagagttaatggaggaatttatgattgcttatcaagattcccttcttgaaataggtcttgataatagagaaattgcaagaatggctcttgcagcggtgatataa

SEQ ID NO: 55 - protein, 241 aa, Prochlorococcus marinus
MQAFASNNLTVEKEELSSNSLPDFTSESYKDAYSRINAVVIEGEQEAYSNFLDLAKLIPEHADELVRLGKMEKKHMNGFCACGRNLAVKPDMPFAKTFFSKLHNNFLEAFKVGDTTTCLLIQCILIESFAISAYHVYIRVADPFAKRITEGVVQDEYLHLNYGQEWLKANLETVKKDLMRANKENLPLIKSMLDEVSNDAEVLHMDKEELMEEFMIAYQDSLLEIGLDNREIARMALAAVI

SEQ ID NO: 56 - DNA, 732 bp, Synechococcus sp.
atgccgacccttgagacgtctgaggtcgccgttcttgaagactcgatggcttcaggctcccggctgcctgatttcaccagcgaggcttacaaggacgcctacagccgcatcaatgcgatcgtgatcgagggtgagcaggaagcgcacgacaactacatcgccctcggcacgctgatccccgagcagaaggatgagctggcccgtctcgcccgcatggagatgaagcacatgaaggggttcacctcctgtggccgcaatctcggcgtggaggcagaccttccctttgctaaggaattcttcgcccccctgcacgggaacttccaggcagctctccaggagggcaaggtggtgacctgcctgttgattcaggcgctgctgattgaagcgttcgccatttccgcctatcacatctacatcccggtggcggatcccttcgctcgcaagatcactgaaggtgtggtgaaggatgagtacacccacctcaattacggccaggaatggctgaaggccaattttgaggccagcaaggatgagctgatggaggccaacaaggccaatctgcctctgatccgctcgatgctggagcaggtggcagccgacgccgccgtgctgcagatggaaaaggaagatctgatcgaagatttcctgatcgcttaccaggaggccctctgcgagatcggtttcagctcccgtgacattgctcgcatggccgccgctgccctcgcggtctga

SEQ ID NO: 57 - protein, 243 aa, Synechococcus sp.
MPTLETSEVAVLEDSMASGSRLPDFTSEAYKDAYSRINAIVIEGEQEAHDNYIALGTLIPEQKDELARLARMEMKHMKGFTSCGRNLGVEADLPFAKEFFAPLHGNFQAALQEGKVVTCLLIQALLIEAFAISAYHIYIPVADPFARKITEGVVKDEYTHLNYGQEWLKANFEASKDELMEANKANLPLIRSMLEQVAADAAVLQMEKEDLIEDFLIAYQEALCEIGFSSRDIARMAAAALAV

SEQ ID NO: 58 - DNA, 681 bp, Synechococcus sp.
atgacccagctcgactttgccagtgcggcctaccgcgaggcctacagccggatcaacggcgttgtgattgtgggcgaaggtctcgccaatcgccatttccagatgttggcgcggcgcattcccgctgatcgcgacgagctgcagcggctcggacgcatggagggagaccatgccagcgcctttgtgggctgtggtcgcaacctcggtgtggtggccgatctgcccctggcccggcgcctgtttcagcccctccatgatctgttcaaacgccacgaccacgacggcaatcgggccgaatgcctggtgatccaggggttgatcgtggaatgtttcgccgtggcggcttaccgccactacctgccggtggccgatgcctacgcccggccgatcaccgcagcggtgatgaacgatgaatcggaacacctcgactacgctgagacctggctgcagcgccatttcgatcaggtgaaggcccgggtcagcgcggtggtggtggaggcgttgccgctcaccctggcgatgttgcaatcgcttgctgcagacatgcgacagatcggcatggatccggtggagaccctggccagcttcagtgaactgtttcgggaagcgttggaatcggtggggtttgaggctgtggaggccaggcgactgctgatgcgagcggccgcccggatggtctga

SEQ ID NO: 59 - protein, 226 aa, Synechococcus sp.
MTQLDFASAAYREAYSRINGVVIVGEGLANRHFQMLARRIPADRDELQRLGRMEGDHASAFVGCGRNLGVVADLPLARRLFQPLHDLFKRHDHDGNRAECLVIQGLIVECFAVAAYRHYLPVADAYARPITAAVMNDESEHLDYAETWLQRHFDQVKARVSAVVVEALPLTLAMLQSLAADMRQIGMDPVETLASFSELFREALESVGFEAVEARRLLMRAAARMV

SEQ ID NO: 60 - DNA, 696 bp, Cyanothece sp.
atgcaagagcttgctttacgctcagagcttgattttaacagcgaaacctataaagatgcttacagtcgcatcaatgctattgtcattgaaggggaacaagaagcctatcaaaattatcttgatatggcgcaacttctcccagaagacgaggctgagttaattcgtctctccaagatggaaaaccgtcacaaaaaaggctttcaagcctgtggcaagaatttgaatgtgaccccagatatggactacgctcaacaattttttgctgaacttcatggcaacttccaaaaggcaaaagccgaaggcaaaattgtcacttgcttattaattcaatctttgatcatcgaagcctttgcgatcgccgcttataatatttatattcctgtggcagatccctttgctcgtaaaatcaccgaaggggtagttaaggatgaatatacccacctcaattttggggaagtctggttaaaagagcattttgaagcctctaaagcagaattagaagacgcaaataaagaaaatttaccccttgtttggcaaatgctcaaccaagttgaaaaagatgccgaagtgttagggatggagaaagaagccttagtggaagatttcatgattagttatggagaagctttaagtaatattggtttctctacccgtgagatcatgaaaatgtctgcttacgggctacgggctgcttaa

SEQ ID NO: 61 - protein, 231 aa, Cyanothece sp.
MQELALRSELDFNSETYKDAYSRINAIVIEGEQEAYQNYLDMAQLLPEDEAELIRLSKMENRHKKGFQACGKNLNVTPDMDYAQQFFAELHGNFQKAKAEGKIVTCLLIQSLIIEAFAIAAYNIYIPVADPFARKITEGVVKDEYTHLNFGEVWLKEHFEASKAELEDANKENLPLVWQMLNQVEKDAEVLGMEKEALVEDFMISYGEALSNIGFSTREIMKMSAYGLRAA

SEQ ID NO: 62 - DNA, 696 bp, Cyanothece sp.
atgcctcaagtgcagtccccatcggctatagacttctacagtgagacctaccaggatgcttacagccgcattgatgcgatcgtgatcgagggagaacaggaagcccacgacaattacctgaagctgacggaactgctgccggattgtcaagaagatctggtccggctggccaaaatggaagcccgtcacaaaaaagggtttgaagcttgtggccgcaatctcaaggtcacacccgatatggagtttgctcaacagttctttgctgacctgcacaacaatttccagaaagctgctgcggccaacaaaattgccacctgtctggtgatccaggccctgattattgagtgctttgccatcgccgcttataacatctatattcctgtcgctgatgactttgcccgcaaaattaccgaaaacgtggtcaaagacgaatacacccacctcaactttggtgaagagtggctcaaagctaactttgatagccagcgggaagaagtggaagcggccaaccgggaaaacctgccgatcgtctggcggatgctcaatcaggtagagactgatgctcacgttttaggtatggaaaaagaggctttagtggaaagcttcatgatccaatatggtgaagccctggaaaatattggtttctcgacccgtgagatcatgcgcatgtccgtttacggcctctctgcggcataa

SEQ ID NO: 63 - DNA, 702 bp, Cyanothece sp.
atgtctgattgcgccacgaacccagccctcgactattacagtgaaacctaccgcaatgcttaccggcgggtgaacggtattgtgattgaaggcgagaagcaagcctacgacaactttatccgcttagctgagctgctcccagagtatcaagcggaattaacccgtctggctaaaatggaagcccgccaccagaagagctttgttgcctgtggccaaaatctcaaggttagcccggacttagactttgcggcacagttttttgctgaactgcatcaaatttttgcatctgcagcaaatgcgggccaggtggctacctgtctggttgtgcaagccctgatcattgaatgctttgcgatcgccgcctacaatacctatttgccagtagcggatgaatttgcccgtaaagtcaccgcatccgttgttcaggacgagtacagccacctaaactttggtgaagtctggctgcagaatgcgtttgagcagtgtaaagacgaaattatcacagctaaccgtcttgctctgccgctgatctggaaaatgctcaaccaggtgacaggcgaattgcgcattctgggcatggacaaagcttctctggtagaagactttagcactcgctatggagaggccctgggccagattggtttcaaactatctgaaattctctccctgtccgttcagggtttacaggcggttacgccttag

SEQ ID NO: 64 - DNA, 702 bp, Cyanothece sp.; identical to SEQ ID NO: 63

SEQ ID NO: 65 - protein, 233 aa, Cyanothece sp.
MSDCATNPALDYYSETYRNAYRRVNGIVIEGEKQAYDNFIRLAELLPEYQAELTRLAKMEARHQKSFVACGQNLKVSPDLDFAAQFFAELHQIFASAANAGQVATCLVVQALIIECFAIAAYNTYLPVADEFARKVTASVVQDEYSHLNFGEVWLQNAFEQCKDEIITANRLALPLIWKMLNQVTGELRILGMDKASLVEDFSTRYGEALGQIGFKLSEILSLSVQGLQAVTP

SEQ ID NO: 66 - DNA, 696 bp, Anabaena variabilis
atgcagcaggttgcagccgatttagaaatcgatttcaagagcgaaaaatataaagatgcctatagtcgcataaatgcgatcgtgattgaaggggaacaagaagcatatgagaattacattcaactatcccaactgctgccagacgataaagaagacctaattcgcctctcgaaaatggaaagtcgccacaaaaaaggatttgaagcttgtggacggaacctgcaagtatccccagacatagagttcgctaaagaattctttgccgggctacacggtaatttccaaaaagcggcagctgaaggtaaagttgtcacttgcctattgattcaatccctgattattgaatgttttgcgatcgccgcatacaatatctacatccccgtggctgacgatttcgcccgtaaaatcactgagggtgtagttaaagatgaatacagtcacctcaacttcggcgaagtttggttacagaaaaatttcgctcaatcaaaagcagaactagaagaagctaatcgtcataatcttcccatagtctggaaaatgctcaatcaagttgccgatgatgcggcagtcttagctatggaaaaagaagccctagtggaagattttatgattcagtacggcgaagcactaagtaatattggcttcacaaccagagatattatgcggatgtcagcctacggactcacagcagcttaa

SEQ ID NO: 67 - protein, 231 aa, Anabaena variabilis
MQQVAADLEIDFKSEKYKDAYSRINAIVIEGEQEAYENYIQLSQLLPDDKEDLIRLSKMESRHKKGFEACGRNLQVSPDIEFAKEFFAGLHGNFQKAAAEGKVVTCLLIQSLIIECFAIAAYNIYIPVADDFARKITEGVVKDEYSHLNFGEVWLQKNFAQSKAELEEANRHNLPIVWKMLNQVADDAAVLAMEKEALVEDFMIQYGEALSNIGFTTRDIMRMSAYGLTAA

SEQ ID NO: 68 - DNA, 765 bp, Synechococcus elongatus
gtgcgtaccccctgggatccaccaaatcccacattctccctctcatccgtgtcaggagaccgcagactcatgccgcagcttgaagccagccttgaactggactttcaaagcgagtcctacaaagacgcttacagccgcatcaacgcgatcgtgattgaaggcgaacaagaggcgttcgacaactacaatcgccttgctgagatgctgcccgaccagcgggatgagcttcacaagctagccaagatggaacagcgccacatgaaaggctttatggcctgtggcaaaaatctctccgtcactcctgacatgggttttgcccagaaatttttcgagcgcttgcacgagaacttcaaagcggcggctgcggaaggcaaggtcgtcacctgcctactgattcaatcgctaatcatcgagtgctttgcgatcgcggcttacaacatctacatcccagtggcggatgcttttgcccgcaaaatcacggagggggtcgtgcgcgacgaatacctgcaccgcaacttcggtgaagagtggctgaaggcgaattttgatgcttccaaagccgaactggaagaagccaatcgtcagaacctgcccttggtttggctaatgctcaacgaagtggccgatgatgctcgcgaactcgggatggagcgtgagtcgctcgtcgaggactttatgattgcctacggtgaagctctggaaaacatcggcttcacaacgcgcgaaatcatgcgtatgtccgcctatggccttgcggccgtttga

SEQ ID NO: 69 - protein, 254 aa, Synechococcus elongatus
MRTPWDPPNPTFSLSSVSGDRRLMPQLEASLELDFQSESYKDAYSRINAIVIEGEQEAFDNYNRLAEMLPDQRDELHKLAKMEQRHMKGFMACGKNLSVTPDMGFAQKFFERLHENFKAAAAEGKVVTCLLIQSLIIECFAIAAYNIYIPVADAFARKITEGVVRDEYLHRNFGEEWLKANFDASKAELEEANRQNLPLVWLMLNEVADDARELGMERESLVEDFMIAYGEALENIGFTTREIMRMSAYGLAAV

SEQ ID NO: 70 - DNA, 1026 bp, Synechococcus elongatus
atgttcggtcttatcggtcatctcaccagtttggagcaggcccgcgacgtttctcgcaggatgggctacgacgaatacgccgatcaaggattggagttttggagtagcgctcctcctcaaatcgttgatgaaatcacagtcaccagtgccacaggcaaggtgattcacggtcgctacatcgaatcgtgtttcttgccggaaatgctggcggcgcgccgcttcaaaacagccacgcgcaaagttctcaatgccatgtcccatgcccaaaaacacggcatcgacatctcggccttggggggctttacctcgattattttcgagaatttcgatttggccagtttgcggcaagtgcgcgacactaccttggagtttgaacggttcaccaccggcaatactcacacggcctacgtaatctgtagacaggtggaagccgctgctaaaacgctgggcatcgacattacccaagcgacagtagcggttgtcggcgcgactggcgatatcggtagcgctgtctgccgctggctcgacctcaaactgggtgtcggtgatttgatcctgacggcgcgcaatcaggagcgtttggataacctgcaggctgaactcggccggggcaagattctgcccttggaagccgctctgccggaagctgactttatcgtgtgggtcgccagtatgcctcagggcgtagtgatcgacccagcaaccctgaagcaaccctgcgtcctaatcgacgggggctaccccaaaaacttgggcagcaaagtccaaggtgagggcatctatgtcctcaatggcggggtagttgaacattgcttcgacatcgactggcagatcatgtccgctgcagagatggcgcggcccgagcgccagatgtttgcctgctttgccgaggcgatgctcttggaatttgaaggctggcatactaacttctcctggggccgcaaccaaatcacgatcgagaagatggaagcgatcggtgaggcatcggtgcgccacggcttccaacccttggcattggcaatttga

SEQ ID NO: 71 - protein, 341 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALAI

SEQ ID NO: 72 - DNA, 1023 bp, Synechocystis sp.
atgtttggtcttattggtcatctcacgagtttagaacacgcccaagcggttgctgaagatttaggctatcctgagtacgccaaccaaggcctggatttttggtgttcggctcctccccaagtggttgataattttcaggtgaaaagtgtgacggggcaggtgattgaaggcaaatatgtggagtcttgctttttgccggaaatgttaacccaacggcggatcaaagcggccattcgtaaaatcctcaatgctatggccctggcccaaaaggtgggcttggatattacggccctgggaggcttttcttcaatcgtatttgaagaatttaacctcaagcaaaataatcaagtccgcaatgtggaactagattttcagcggttcaccactggtaatacccacaccgcttatgtgatctgccgtcaggtcgagtctggagctaaacagttgggtattgatctaagtcaggcaacggtagcggtttgtggcgccacgggagatattggtagcgccgtatgtcgttggttagatagcaaacatcaagttaaggaattattgctaattgcccgtaaccgccaaagattggaaaatctccaagaggaattgggtcggggcaaaattatggatttggaaacagccctgccccaggcagatattattgtttgggtggctagtatgcccaagggggtagaaattgcgggggaaatgctgaaaaagccctgtttgattgtggatgggggctatcccaagaatttagacaccagggtgaaagcggatggggtgcatattctcaagggggggattgtagaacattcccttgatattacctgggaaattatgaagattgtggagatggatattccctcccggcaaatgttcgcctgttttgcggaggccattttgctagagtttgagggctggcgcactaatttttcctggggccgcaaccaaatttccgttaataaaatggaggcgattggtgaagcttctgtcaagcatggcttttgccctttagtagctctttag

SEQ ID NO: 73 - protein, 340 aa, Synechocystis sp.
MFGLIGHLTSLEHAQAVAEDLGYPEYANQGLDFWCSAPPQVVDNFQVKSVTGQVIEGKYVESCFLPEMLTQRRIKAAIRKILNAMALAQKVGLDITALGGFSSIVFEEFNLKQNNQVRNVELDFQRFTTGNTHTAYVICRQVESGAKQLGIDLSQATVAVCGATGDIGSAVCRWLDSKHQVKELLLIARNRQRLENLQEELGRGKIMDLETALPQADIIVWVASMPKGVEIAGEMLKKPCLIVDGGYPKNLDTRVKADGVHILKGGIVEHSLDITWEIMKIVEMDIPSRQMFACFAEAILLEFEGWRTNFSWGRNQISVNKMEAIGEASVKHGFCPLVAL

SEQ ID NO: 74 - DNA, 1023 bp, Cyanothece sp.
atgtttggtttaattggtcatcttacaagtttagaacacgcccactccgttgctgatgcctttggctatggcccatacgccactcagggacttgatttgtggtgttctgctccaccccaattcgtcgagcattttcatgttactagcatcacaggacaaaccatcgaaggaaagtatatagaatccgctttcttaccagaaatgctgataaagcgacggattaaagcagcaattcgcaaaatactgaatgcgatggcctttgctcagaaaaataaccttaacatcacagcattagggggcttttcttcgattatttttgaagaatttaatctcaaagagaatagacaagttcgtaatgtctctttagagtttgatcgcttcaccaccggaaacacccatactgcttatatcatttgtcgtcaagttgaacaggcatccgctaaactagggattgacttatcccaagcaacggttgctatttgcggggcaaccggagatattggcagtgcagtgtgtcgttggttagatagaaaaaccgatacccaggaactattcttaattgctcgcaataaagaacgattacaacgactgcaagatgagttgggacggggtaaaattatgggattggaggaggctttacccgaagcagatattatcgtttgggtggcgagtatgcccaaaggagtggaaattaatgccgaaactctcaaaaaaccctgtttaattatcgatggtggttatcctaagaatttagacacaaaaattaaacatcctgatgtccatatcctgaaagggggaattgtagaacattctctagatattgactggaagattatggaaactgtcaatatggatgttccttctcgtcaaatgtttgcttgttttgccgaagccattttattagagtttgaacaatggcacactaatttttcttggggacgcaatcaaattacagtgactaaaatggaacaaataggagaagcttctgtcaaacatgggttacaaccgttgttgagttggtaa

SEQ ID NO: 75 - protein, 340 aa, Cyanothece sp.
MFGLIGHLTSLEHAHSVADAFGYGPYATQGLDLWCSAPPQFVEHFHVTSITGQTIEGKYIESAFLPEMLIKRRIKAAIRKILNAMAFAQKNNLNITALGGFSSIIFEEFNLKENRQVRNVSLEFDRFTTGNTHTAYIICRQVEQASAKLGIDLSQATVAICGATGDIGSAVCRWLDRKTDTQELFLIARNKERLQRLQDELGRGKIMGLEEALPEADIIVWVASMPKGVEINAETLKKPCLIIDGGYPKNLDTKIKHPDVHILKGGIVEHSLDIDWKIMETVNMDVPSRQMFACFAEAILLEFEQWHTNFSWGRNQITVTKMEQIGEASVKHGLQPLLSW

SEQ ID NO: 76 - DNA, 1041 bp, Prochlorococcus marinus
atgtttgggcttataggtcattcaactagttttgaagatgcaaaaagaaaggcttcattattgggctttgatcatattgcggatggtgatttagatgtttggtgcacagctccacctcaactagttgaaaatgtagaggttaaaagtgctataggtatatcaattgaaggttcttatattgattcatgtttcgttcctgaaatgctttcaagatttaaaacggcaagaagaaaagtattaaatgcaatggaattagctcaaaaaaaaggtattaatattaccgctttgggggggttcacttctatcatctttgaaaattttaatctccttcaacataagcagattagaaacacttcactagagtgggaaaggtttacaactggtaatactcatactgcgtgggttatttgcaggcaattagagatgaatgctcctaaaataggtattgatcttaaaagcgcaacagttgctgtagttggtgctactggagatataggcagtgctgtttgtcgatggttaatcaataaaacaggtattggggaacttcttttggtagctaggcaaaaggaacccttggattctttgcaaaaggaattagatggtggaactatcaaaaatctagatgaagcattgcctgaagcagatattgttgtatgggtagcaagtatgccaaagacaatggaaatcgatgctaataatcttaaacaaccatgtttaatgattgatggaggttatccaaagaatctagatgaaaaatttcaaggaaataatatacatgttgtaaaaggaggtatagtaagattcttcaatgatataggttggaatatgatggaactagctgaaatgcaaaatccccagagagaaatgtttgcatgctttgcagaagcaatgattttagaatttgaaaaatgtcatacaaactttagctggggaagaaataatatatctctcgagaaaatggagtttattggagctgcttctgtaaagcatggcttctctgcaattggcctagataagcatccaaaagtactagcagtttga

SEQ ID NO: 77 - protein, 346 aa, Prochlorococcus marinus
MFGLIGHSTSFEDAKRKASLLGFDHIADGDLDVWCTAPPQLVENVEVKSAIGISIEGSYIDSCFVPEMLSRFKTARRKVLNAMELAQKKGINITALGGFTSIIFENFNLLQHKQIRNTSLEWERFTTGNTHTAWVICRQLEMNAPKIGIDLKSATVAVVGATGDIGSAVCRWLINKTGIGELLLVARQKEPLDSLQKELDGGTIKNLDEALPEADIVVWVASMPKTMEIDANNLKQPCLMIDGGYPKNLDEKFQGNNIHVVKGGIVRFFNDIGWNMMELAEMQNPQREMFACFAEAMILEFEKCHTNFSWGRNNISLEKMEFIGAASVKHGFSAIGLDKHPKVLAV

SEQ ID NO: 78 - DNA, 1053 bp, Gloeobacter violaceus
atgtttggcctgatcggacacttgaccaatctttcccatgcccagcgggtcgcccgcgacctgggctacgacgagtatgcaagccacgacctcgaattctggtgcatggcccctccccaggcggtcgatgaaatcacgatcaccagcgtcaccggtcaggtgatccacggtcagtacgtcgaatcgtgctttctgccggagatgctcgcccagggccgcttcaagaccgccatgcgcaagatcctcaatgccatggccctggtccagaagcgcggcatcgacattacggccctgggaggcttctcgtcgatcatcttcgagaatttcagcctcgataaattgctcaacgtccgcgacatcaccctcgacatccagcgcttcaccaccggcaacacccacacggcctacatcctttgtcagcaggtcgagcagggtgcggtacgctacggcatcgatccggccaaagcgaccgtggcggtagtcggggccaccggcgacatcggtagcgccgtctgccgatggctcaccgaccgcgccggcatccacgaactcttgctggtggcccgcgacgccgaaaggctcgaccggctgcagcaggaactcggcaccggtcggatcctgccggtcgaagaagcacttcccaaagccgacatcgtcgtctgggtcgcctcgatgaaccagggcatggccatcgaccccgccggcctgcgcaccccctgcctgctcatcgacggcggctaccccaagaacatggccggcaccctgcagcgcccgggcatccatatcctcgacggcggcatggtcgagcactcgctcgacatcgactggcagatcatgtcgtttctaaatgtgcccaaccccgcccgccagttcttcgcctgcttcgccgagtcgatgctgctggaattcgaagggcttcacttcaatttttcctggggccgcaaccacatcaccgtcgagaagatggcccagatcggctcgctgtctaaaaaacatggctttcgtcccctgcttgaacccagtcagcgcagcggcgaactcgtacacggataa

SEQ ID NO: 79 - protein, 350 aa, Gloeobacter violaceus
MFGLIGHLTNLSHAQRVARDLGYDEYASHDLEFWCMAPPQAVDEITITSVTGQVIHGQYVESCFLPEMLAQGRFKTAMRKILNAMALVQKRGIDITALGGFSSIIFENFSLDKLLNVRDITLDIQRFTTGNTHTAYILCQQVEQGAVRYGIDPAKATVAVVGATGDIGSAVCRWLTDRAGIHELLLVARDAERLDRLQQELGTGRILPVEEALPKADIVVWVASMNQGMAIDPAGLRTPCLLIDGGYPKNMAGTLQRPGIHILDGGMVEHSLDIDWQIMSFLNVPNPARQFFACFAESMLLEFEGLHFNFSWGRNHITVEKMAQIGSLSKKHGFRPLLEPSQRSGELVHG

SEQ ID NO: 80 - DNA, 1020 bp, Nostoc punctiforme
atgtttggtctaattggacatctgactagtttagaacacgctcaagccgtagcccaagaattgggatacccagaatatgccgatcaagggctagacttttggtgcagcgccccgccgcaaattgtcgatagtattattgtcaccagtgttactgggcaacaaattgaaggacgatatgtagaatcttgctttttgccggaaatgctagctagtcgccgcatcaaagccgcaacacggaaaatcctcaacgctatggcccatgcacagaagcacggcattaacatcacagctttaggcggattttcctcgattatttttgaaaactttaagttagagcagtttagccaagtccgaaatatcaagctagagtttgaacgcttcaccacaggaaacacgcatactgcctacattatttgtaagcaggtggaagaagcatccaaacaactgggaattaatctatcaaacgcgactgttgcggtatgtggagcaactggggatattggtagtgccgttacacgctggctagatgcgagaacagatgtccaagaactcctgctaatcgcccgcgatcaagaacgtctcaaagagttgcaaggcgaactggggcgggggaaaatcatgggtttgacagaagcactaccccaagccgatgttgtagtttgggttgctagtatgcccagaggcgtggaaattgaccccaccactttgaaacaaccctgtttgttgattgatggtggctatcctaaaaacttagcaacaaaaattcaatatcctggcgtacacgtgttaaatggtgggattgtagagcattccctggatattgactggaaaattatgaaaatagtcaatatggacgtgccagcccgtcagttgtttgcctgttttgccgaatcaatgctactggaatttgagaagttatacacgaacttttcgtggggacggaatcagattaccgtagataaaatggagcagattggccgggtgtcagtaaaacatggatttagaccgttgttggtttag

SEQ ID NO: 81 - protein, 339 aa, Nostoc punctiforme
MFGLIGHLTSLEHAQAVAQELGYPEYADQGLDFWCSAPPQIVDSIIVTSVTGQQIEGRYVESCFLPEMLASRRIKAATRKILNAMAHAQKHGINITALGGFSSIIFENFKLEQFSQVRNIKLEFERFTTGNTHTAYIICKQVEEASKQLGINLSNATVAVCGATGDIGSAVTRWLDARTDVQELLLIARDQERLKELQGELGRGKIMGLTEALPQADVVVWVASMPRGVEIDPTTLKQPCLLIDGGYPKNLATKIQYPGVHVLNGGIVEHSLDIDWKIMKIVNMDVPARQLFACFAESMLLEFEKLYTNFSWGRNQITVDKMEQIGRVSVKHGFRPLLV

SEQ ID NO: 82 - DNA, 1020 bp, Anabaena variabilis
atgtttggtctaattggacatctgacaagtttagaacacgctcaagcggtagctcaagaactgggatacccagaatacgccgaccaagggctagatttttggtgcagcgctccaccgcaaatagttgaccacattaaagttactagcattactggtgaaataattgaagggaggtatgtagaatcttgctttttaccagaaatgctagccagccgtaggattaaagccgcaacccgcaaagtcctcaatgctatggctcatgctcaaaaacatggcattgacatcaccgctttgggtggtttctcctccattatttttgaaaacttcaaattggaacagtttagccaagttcgtaatgtcacactagagtttgaacgcttcactacaggcaacactcacacagcttatatcatttgtcggcaggtagaacaagcatcacaacaactcggcattgaactctcccaagcaacagtagctatatgtggggctactggtgacattggtagtgcagttactcgctggctggatgccaaaacagacgtaaaagaattactgttaatcgcccgtaatcaagaacgtctccaagagttgcaaagcgagttgggacgcggtaaaatcatgagcctagatgaagcattgcctcaagctgatattgtagtttgggtagctagtatgcctaaaggcgtggaaattaatcctcaagttttgaaacaaccctgtttattgattgatggtggttatccgaaaaacttgggtacaaaagttcagtatcctggtgtttatgtactgaacggaggtatcgtcgaacattccctagatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 83 - protein, 339 aa, Anabaena variabilis
MFGLIGHLTSLEHAQAVAQELGYPEYADQGLDFWCSAPPQIVDHIKVTSITGEIIEGRYVESCFLPEMLASRRIKAATRKVLNAMAHAQKHGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 84 - DNA, 1026 bp, Synechococcus elongatus; identical to SEQ ID NO: 70

SEQ ID NO: 85 - protein, 340 aa, Synechococcus elongatus
MFGLIGHLTSLEQARDVSRRMGYDEYADQGLEFWSSAPPQIVDEITVTSATGKVIHGRYIESCFLPEMLAARRFKTATRKVLNAMSHAQKHGIDISALGGFTSIIFENFDLASLRQVRDTTLEFERFTTGNTHTAYVICRQVEAAAKTLGIDITQATVAVVGATGDIGSAVCRWLDLKLGVGDLILTARNQERLDNLQAELGRGKILPLEAALPEADFIVWVASMPQGVVIDPATLKQPCVLIDGGYPKNLGSKVQGEGIYVLNGGVVEHCFDIDWQIMSAAEMARPERQMFACFAEAMLLEFEGWHTNFSWGRNQITIEKMEAIGEASVRHGFQPLALA

SEQ ID NO: 86 - DNA, 1020 bp, Nostoc sp.
atgtttggtctaattggacatctgacaagtttagaacacgctcaagcggtagctcaagaactgggatacccagaatacgccgaccaagggctagatttttggtgtagcgctccaccgcaaatagttgaccacattaaagttactagtattactggtgaaataattgaagggaggtatgtagaatcttgctttttaccggagatgctagccagtcgtcggattaaagccgcaacccgcaaagtcctcaatgctatggctcatgctcaaaagaatggcattgatatcacagctttgggtggtttctcctccattatttttgaaaactttaaattggagcagtttagccaagttcgtaatgtgacactagagtttgaacgcttcactacaggcaacactcacacagcatatattatttgtcggcaggtagaacaagcatcacaacaactcggcattgaactctcccaagcaacagtagctatatgtggggctactggtgatattggtagtgcagttactcgctggctggatgctaaaacagacgtgaaagaattgctgttaatcgcccgtaatcaagaacgtctccaagagttgcaaagcgagctgggacgcggtaaaatcatgagccttgatgaagcactgccccaagctgatatcgtagtttgggtagccagtatgcctaaaggtgtggaaattaatcctcaagttttgaagcaaccctgtttgctgattgatgggggttatccgaaaaacttgggtacaaaagttcagtatcctggtgtttatgtactgaacggcggtatcgtcgaacattcgctggatattgactggaaaatcatgaaaatagtcaatatggatgtacctgcacgccaattatttgcttgttttgcggaatctatgctcttggaatttgagaagttgtacacgaacttttcttgggggcgcaatcagattaccgtagacaaaatggagcagattggtcaagcatcagtgaaacatgggtttagaccactgctggtttag

SEQ ID NO: 87 - protein, 339 aa, Nostoc sp.
MFGLIGHLTSLEHAQAVAQELGYPEYADQGLDFWCSAPPQIVDHIKVTSITGEIIEGRYVESCFLPEMLASRRIKAATRKVLNAMAHAQKNGIDITALGGFSSIIFENFKLEQFSQVRNVTLEFERFTTGNTHTAYIICRQVEQASQQLGIELSQATVAICGATGDIGSAVTRWLDAKTDVKELLLIARNQERLQELQSELGRGKIMSLDEALPQADIVVWVASMPKGVEINPQVLKQPCLLIDGGYPKNLGTKVQYPGVYVLNGGIVEHSLDIDWKIMKIVNMDVPARQLFACFAESMLLEFEKLYTNFSWGRNQITVDKMEQIGQASVKHGFRPLLV

SEQ ID NO: 88 - DNA, 3522 bp, Mycobacterium smegmatis
atgaccagcgatgttcacgacgccacagacggcgtcaccgaaaccgcactcgacgacgagcagtcgacccgccgcatcgccgagctgtacgccaccgatcccgagttcgccgccgccgcaccgttgcccgccgtggtcgacgcggcgcacaaacccgggctgcggctggcagagatcctgcagaccctgttcaccggctacggtgaccgcccggcgctgggataccgcgcccgtgaactggccaccgacgagggcgggcgcaccgtgacgcgtctgctgccgcggttcgacaccctcacctacgcccaggtgtggtcgcgcgtgcaagcggtcgccgcggccctgcgccacaacttcgcgcagccgatctaccccggcgacgccgtcgcgacgatcggtttcgcgagtcccgattacctgacgctggatctcgtatgcgcctacctgggcctcgtgagtgttccgctgcagcacaacgcaccggtcagccggctcgccccgatcctggccgaggtcgaaccgcggatcctcaccgtgagcgccgaatacctcgacctcgcagtcgaatccgtgcgggacgtcaactcggtgtcgcagctcgtggtgttcgaccatcaccccgaggtcgacgaccaccgcgacgcactggcccgcgcgcgtgaacaactcgccggcaagggcatcgccgtcaccaccctggacgcgatcgccgacgagggcgccgggctgccggccgaaccgatctacaccgccgaccatgatcagcgcctcgcgatgatcctgtacacctcgggttccaccggcgcacccaagggtgcgatgtacaccgaggcgatggtggcgcggctgtggaccatgtcgttcatcacgggtgaccccacgccggtcatcaacgtcaacttcatgccgctcaaccacctgggcgggcgcatccccatttccaccgccgtgcagaacggtggaaccagttacttcgtaccggaatccgacatgtccacgctgttcgaggatctcgcgctggtgcgcccgaccgaactcggcctggttccgcgcgtcgccgacatgctctaccagcaccacctcgccaccgtcgaccgcctggtcacgcagggcgccgacgaactgaccgccgagaagcaggccggtgccgaactgcgtgagcaggtgctcggcggacgcgtgatcaccggattcgtcagcaccgcaccgctggccgcggagatgagggcgttcctcgacatcaccctgggcgcacacatcgtcgacggctacgggctcaccgagaccggcgccgtgacacgcgacggtgtgatcgtgcggccaccggtgatcgactacaagctgatcgacgttcccgaactcggctacttcagcaccgacaagccctacccgcgtggcgaactgctggtcaggtcgcaaacgctgactcccgggtactacaagcgccccgaggtcaccgcgagcgtcttcgaccgggacggctactaccacaccggcgacgtcatggccgagaccgcacccgaccacctggtgtacgtggaccgtcgcaacaacgtcctcaaactcgcgcagggcgagttcgtggcggtcgccaacctggaggcggtgttctccggcgcggcgctggtgcgccagatcttcgtgtacggcaacagcgagcgcagtttccttctggccgtggtggtcccgacgccggaggcgctcgagcagtacgatccggccgcgctcaaggccgcgctggccgactcgctgcagcgcaccgcacgcgacgccgaactgcaatcctacgaggtgccggccgatttcatcgtcgagaccgagccgttcagcgccgccaacgggctgctgtcgggtgtcggaaaactgctgcggcccaacctcaaagaccgctacgggcagcgcctggagcagatgtacgccgatatcgcggccacgcaggccaaccagttgcgcgaactgcggcgcgcggccgccacacaaccggtgatcgacaccctcacccaggccgctgccacgatcctcggcaccgggagcgaggtggcatccgacgcccacttcaccgacctgggcggggattccctgtcggcgctgacactttcgaacctgctgagcgatttcttcggtttcgaagttcccgtcggcaccatcgtgaacccggccaccaacctcgcccaactcgcccagcacatcgaggcgcagcgcaccgcgggtgaccgcaggccgagtttcaccaccgtgcacggcgcggacgccaccgagatccgggcgagtgagctgaccctggacaagttcatcgacgccgaaacgctccgggccgcaccgggtctgcccaaggtcaccaccgagccacggacggtgttgctctcgggcgccaacggctggctgggccggttcctcacgttgcagtggctggaacgcctggcacctgtcggcggcaccctcatcacgatcgtgcggggccgcgacgacgccgcggcccgcgcacggctgacccaggcctacgacaccgatcccgagttgtcccgccgcttcgccgagctggccgaccgccacctgcgggtggtcgccggtgacatcggcgacccgaatctgggcctcacacccgagatctggcaccggctcgccgccgaggtcgacctggtggtgcatccggcagcgctggtcaaccacgtgctcccctaccggcagctgttcggccccaacgtcgtgggcacggccgaggtgatcaagctggccctcaccgaacggatcaagcccgtcacgtacctgtccaccgtgtcggtggccatggggatccccgacttcgaggaggacggcgacatccggaccgtgagcccggtgcgcccgctcgacggcggatacgccaacggctacggcaacagcaagtgggccggcgaggtgctgctgcgggaggcccacgatctgtgcgggctgcccgtggcgacgttccgctcggacatgatcctggcgcatccgcgctaccgcggtcaggtcaacgtgccagacatgttcacgcgactcctgttgagcctcttgatcaccggcgtcgcgccgcggtcgttctacatcggagacggtgagcgcccgcgggcgcactaccccggcctgacggtcgatttcgtggccgaggcggtcacgacgctcggcgcgcagcagcgcgagggatacgtgtcctacgacgtgatgaacccgcacgacgacgggatctccctggatgtgttcgtggactggctgatccgggcgggccatccgatcgaccgggtcgacgactacgacgactgggtgcgtcggttcgagaccgcgttgaccgcgcttcccgagaagcgccgcgcacagaccgtactgccgctgctgcacgcgttccgcgctccgcaggcaccgttgcgcggcgcacccgaacccacggaggtgttccacgccgcggtgcgcaccgcgaaggtgggcccgggagacatcccgcacctcgacgaggcgctgatcgacaagtacatacgcgatctgcgtgagttcggtctgatctga

SEQ ID NO: 89 - DNA, 3582 bp, Mycobacterium smegmatis
atgggcagcagccatcatcatcatcatcacagcagcggcctggtgccgcgcggcagccatatgacgagcgatgttcacgacgcgaccgacggcgttaccgagactgcactggatgatgagcagagcactcgtcgtattgcagaactgtacgcaacggacccagagttcgcagcagcagctcctctgccggccgttgtcgatgcggcgcacaaaccgggcctgcgtctggcggaaatcctgcagaccctgttcaccggctacggcgatcgtccggcgctgggctatcgtgcacgtgagctggcgacggacgaaggcggtcgtacggtcacgcgtctgctgccgcgcttcgataccctgacctatgcacaggtgtggagccgtgttcaagcagtggctgcagcgttgcgtcacaatttcgcacaaccgatttacccgggcgacgcggtcgcgactatcggctttgcgagcccggactatttgacgctggatctggtgtgcgcgtatctgggcctggtcagcgttcctttgcagcataacgctccggtgtctcgcctggccccgattctggccgaggtggaaccgcgtattctgacggtgagcgcagaatacctggacctggcggttgaatccgtccgtgatgtgaactccgtcagccagctggttgttttcgaccatcatccggaagtggacgatcaccgtgacgcactggctcgcgcacgcgagcagctggccggcaaaggtatcgcagttacgaccctggatgcgatcgcagacgaaggcgcaggtttgccggctgagccgatttacacggcggatcacgatcagcgtctggccatgattctgtataccagcggctctacgggtgctccgaaaggcgcgatgtacaccgaagcgatggtggctcgcctgtggactatgagctttatcacgggcgacccgaccccggttatcaacgtgaacttcatgccgctgaaccatctgggcggtcgtatcccgattagcaccgccgtgcagaatggcggtaccagctacttcgttccggaaagcgacatgagcacgctgtttgaggatctggccctggtccgccctaccgaactgggtctggtgccgcgtgttgcggacatgctgtaccagcatcatctggcgaccgtggatcgcctggtgacccagggcgcggacgaactgactgcggaaaagcaggccggtgcggaactgcgtgaacaggtcttgggcggtcgtgttatcaccggttttgtttccaccgcgccgttggcggcagagatgcgtgcttttctggatatcaccttgggtgcacacatcgttgacggttacggtctgaccgaaaccggtgcggtcacccgtgatggtgtgattgttcgtcctccggtcattgattacaagctgatcgatgtgccggagctgggttacttctccaccgacaaaccgtacccgcgtggcgagctgctggttcgtagccaaacgttgactccgggttactacaagcgcccagaagtcaccgcgtccgttttcgatcgcgacggctattaccacaccggcgacgtgatggcagaaaccgcgccagaccacctggtgtatgtggaccgccgcaacaatgttctgaagctggcgcaaggtgaatttgtcgccgtggctaacctggaggccgttttcagcggcgctgctctggtccgccagattttcgtgtatggtaacagcgagcgcagctttctgttggctgttgttgtccctaccccggaggcgctggagcaatacgaccctgccgcattgaaagcagccctggcggattcgctgcagcgtacggcgcgtgatgccgagctgcagagctatgaagtgccggcggacttcattgttgagactgagccttttagcgctgcgaacggtctgctgagcggtgttggcaagttgctgcgtccgaatttgaaggatcgctacggtcagcgtttggagcagatgtacgcggacatcgcggctacgcaggcgaaccaattgcgtgaactgcgccgtgctgcggctactcaaccggtgatcgacacgctgacgcaagctgcggcgaccatcctgggtaccggcagcgaggttgcaagcgacgcacactttactgatttgggcggtgattctctgagcgcgctgacgttgagcaacttgctgtctgacttctttggctttgaagtcccggttggcacgattgttaacccagcgactaatctggcacagctggcgcaacatatcgaggcgcagcgcacggcgggtgaccgccgtccatcctttacgacggtccacggtgcggatgctacggaaatccgtgcaagcgaactgactctggacaaattcatcgacgctgagactctgcgcgcagcacctggtttgccgaaggttacgactgagccgcgtacggtcctgttgagcggtgccaatggttggttgggccgcttcctgaccctgcagtggctggaacgtttggcaccggttggcggtaccctgatcaccattgtgcgcggtcgtgacgatgcagcggcacgtgcacgtttgactcaggcttacgatacggacccagagctgtcccgccgcttcgctgagttggcggatcgccacttgcgtgtggtggcaggtgatatcggcgatccgaatctgggcctgaccccggagatttggcaccgtctggcagcagaggtcgatctggtcgttcatccagcggccctggtcaaccacgtcctgccgtaccgccagctgtttggtccgaatgttgttggcaccgccgaagttatcaagttggctctgaccgagcgcatcaagcctgttacctacctgtccacggttagcgtcgcgatgggtattcctgattttgaggaggacggtgacattcgtaccgtcagcccggttcgtccgctggatggtggctatgcaaatggctatggcaacagcaagtgggctggcgaggtgctgctgcgcgaggcacatgacctgtgtggcctgccggttgcgacgtttcgtagcgacatgattctggcccacccgcgctaccgtggccaagtgaatgtgccggacatgttcacccgtctgctgctgtccctgctgatcacgggtgtggcaccgcgttccttctacattggtgatggcgagcgtccgcgtgcacactacccgggcctgaccgtcgattttgttgcggaagcggttactaccctgggtgctcagcaacgtgagggttatgtctcgtatgacgttatgaatccgcacgatgacggtattagcttggatgtctttgtggactggctgattcgtgcgggccacccaattgaccgtgttgacgactatgatgactgggtgcgtcgttttgaaaccgcgttgaccgccttgccggagaaacgtcgtgcgcagaccgttctgccgctgctgcatgcctttcgcgcgccacaggcgccgttgcgtggcgcccctgaaccgaccgaagtgtttcatgcagcggtgcgtaccgctaaagtcggtccgggtgatattccgcacctggatgaagccctgatcgacaagtacatccgtgacctgcgcgagttcggtctgatttag

SEQ ID NO: 90 - protein, 1173 aa, Mycobacterium smegmatis
MTSDVHDATDGVTETALDDEQSTRRIAELYATDPEFAAAAPLPAVVDAAHKPGLRLAEILQTLFTGYGDRPALGYRARELATDEGGRTVTRLLPRFDTLTYAQVWSRVQAVAAALRHNFAQPIYPGDAVATIGFASPDYLTLDLVCAYLGLVSVPLQHNAPVSRLAPILAEVEPRILTVSAEYLDLAVESVRDVNSVSQLVVFDHHPEVDDHRDALARAREQLAGKGIAVTTLDAIADEGAGLPAEPIYTADHDQRLAMILYTSGSTGAPKGAMYTEAMVARLWTMSFITGDPTPVINVNFMPLNHLGGRIPISTAVQNGGTSYFVPESDMSTLFEDLALVRPTELGLVPRVADMLYQHHLATVDRLVTQGADELTAEKQAGAELREQVLGGRVITGFVSTAPLAAEMRAFLDITLGAHIVDGYGLTETGAVTRDGVIVRPPVIDYKLIDVPELGYFSTDKPYPRGELLVRSQTLTPGYYKRPEVTASVFDRDGYYHTGDVMAETAPDHLVYVDRRNNVLKLAQGEFVAVANLEAVFSGAALVRQIFVYGNSERSFLLAVVVPTPEALEQYDPAALKAALADSLQRTARDAELQSYEVPADFIVETEPFSAANGLLSGVGKLLRPNLKDRYGQRLEQMYADIAATQANQLRELRRAAATQPVIDTLTQAAATILGTGSEVASDAHFTDLGGDSLSALTLSNLLSDFFGFEVPVGTIVNPATNLAQLAQHIEAQRTAGDRRPSFTTVHGADATEIRASELTLDKFIDAETLRAAPGLPKVTTEPRTVLLSGANGWLGRFLTLQWLERLAPVGGTLITIVRGRDDAAARARLTQAYDTDPELSRRFAELADRHLRVVAGDIGDPNLGLTPEIWHRLAAEVDLVVHPAALVNHVLPYRQLFGPNVVGTAEVIKLALTERIKPVTYLSTVSVAMGIPDFEEDGDIRTVSPVRPLDGGYANGYGNSKWAGEVLLREAHDLCGLPVATFRSDMILAHPRYRGQVNVPDMFTRLLLSLLITGVAPRSFYIGDGERPRAHYPGLTVDFVAEAVTTLGAQQREGYVSYDVMNPHDDGISLDVFVDWLIRAGHPIDRVDDYDDWVRRFETALTALPEKRRAQTVLPLLHAFRAPQAPLRGAPEPTEVFHAAVRTAKVGPGDIPHLDEALIDKYIRDLREFGLI

SEQ ID NO: 91 - DNA, 3507 bp, Mycobacterium smegmatis
atgacgatcgaaacgcgcgaagaccgcttcaaccggcgcattgaccacttgttcgaaaccgacccgcagttcgccgccgcccgtcccgacgaggcgatcagcgcggctgccgccgatccggagttgcgccttcctgccgcggtcaaacagattctggccggctatgcggaccgccctgcgctgggcaagcgcgccgtcgagttcgtcaccgacgaagaaggccgcaccaccgcgaagctcctgccccgcttcgacaccatcacctaccgtcagctcgcaggccggatccaggccgtgaccaatgcctggcacaaccatccggtgaatgccggtgaccgcgtggccatcctgggtttcaccagtgtcgactacacgacgatcgacatcgccctgctcgaactcggcgccgtgtccgtaccgctgcagaccagtgcgccggtggcccaactgcagccgatcgtcgccgagaccgagcccaaggtgatcgcgtcgagcgtcgacttcctcgccgacgcagtcgctctcgtcgagtccgggcccgcgccgtcgcgactggtggtgttcgactacagccacgaggtcgacgatcagcgtgaggcgttcgaggcggccaagggcaagctcgcaggcaccggcgtcgtcgtcgagacgatcaccgacgcactggaccgcgggcggtcactcgccgacgcaccgctctacgtgcccgacgaggccgacccgctgacccttctcatctacacctccggcagcaccggcactcccaagggcgcgatgtaccccgagtccaagaccgccacgatgtggcaggccgggtccaaggcccggtgggacgagaccctcggcgtgatgccgtcgatcaccctgaacttcatgcccatgagtcacgtcatggggcgcggcatcctgtgcagcacactcgccagcggcggaaccgcgtacttcgccgcacgcagcgacctgtccaccttcctggaggacctcgccctcgtgcggcccacgcagctcaacttcgttcctcgcatctgggacatgctgttccaggagtaccagagccgcctcgacaaccgccgcgccgagggatccgaggaccgagccgaagccgcagtcctcgaagaggtccgcacccaactgctcggcgggcgattcgtttcggccctgaccggatcggctcccatctcggcggagatgaagagctgggtcgaggacctgctcgacatgcatctgctggagggctacggctccaccgaggccggcgcggtgttcatcgacgggcagatccagcgcccgccggtcatcgactacaagctggtcgacgtgcccgatctcggctacttcgccacggaccggccctacccgcgcggcgaacttctggtcaagtccgagcagatgttccccggctactacaagcgtccggagatcaccgccgagatgttcgacgaggacgggtactaccgcaccggcgacatcgtcgccgagctcgggcccgaccatctcgaatacctcgaccgccgcaacaacgtgctgaaactgtcgcagggcgaattcgtcacggtctccaagctggaggcggtgttcggcgacagccccctggtacgccagatctacgtctacggcaacagcgcgcggtcctatctgctggcggtcgtggtcccgaccgaagaggcactgtcacgttgggacggtgacgaactcaagtcgcgcatcagcgactcactgcaggacgcggcacgagccgccggattgcagtcgtatgagatcccgcgtgacttcctcgtcgagacaacacctttcacgctggagaacggcctgctgaccggtatccgcaagctggcccggccgaaactgaaggcgcactacggcgaacgcctcgaacagctctacaccgacctggccgaggggcaggccaacgagttgcgcgagttgcgccgcaacggagccgaccggcccgtggtcgagaccgtcagccgcgccgcggtcgcactgctcggtgcctccgtcacggatctgcggtccgatgcgcacttcaccgatctgggtggagattcgttgtcggccttgagcttctcgaacctgttgcacgagatcttcgatgtcgacgtgccggtcggcgtcatcgtcagcccggccaccgacctggcaggcgtcgcggcctacatcgagggcgaactgcgcggctccaagcgccccacatacgcgtcggtgcacgggcgcgacgccaccgaggtgcgcgcgcgtgatctcgccctgggcaagttcatcgacgccaagaccctgtccgccgcgccgggtctgccgcgttcgggcaccgagatccgcaccgtgctgctgaccggcgccaccgggttcctgggccgctatctggcgctggaatggctggagcgcatggacctggtggacggcaaggtgatctgcctggtgcgcgcccgcagcgacgacgaggcccgggcgcgtctggacgccacgttcgacaccggggacgcgacactgctcgagcactaccgcgcgctggcagccgatcacctcgaggtgatcgccggtgacaagggcgaggccgatctgggtctcgaccacgacacgtggcagcgactggccgacaccgtcgatctgatcgtcgatccggccgccctggtcaatcacgtcctgccgtacagccagatgttcggacccaatgcgctcggcaccgccgaactcatccggatcgcgctgaccaccacgatcaagccgtacgtgtacgtctcgacgatcggtgtgggacagggcatctcccccgaggcgttcgtcgaggacgccgacatccgcgagatcagcgcgacgcgccgggtcgacgactcgtacgccaacggctacggcaacagcaagtgggccggcgaggtcctgctgcgggaggcgcacgactggtgtggtctgccggtctcggtgttccgctgcgacatgatcctggccgacacgacctactcgggtcagctgaacctgccggacatgttcacccgcctgatgctgagcctcgtggcgaccggcatcgcgcccggttcgttctacgaactcgatgcggacggcaaccggcagcgcgcccactacgacgggctgcccgtggagttcatcgccgaggcgatctccaccatcggctcgcaggtcaccgacggattcgagacgttccacgtgatgaacccgtacgacgacggcatcggcctcgacgagtacgtggactggctgatcgaggccggctaccccgtgcaccgcgtcgacgactacgccacctggctgagccggttcgaaaccgcactgcgggccctgccggaacggcaacgtcaggcctcgctgctgccgctgctgcacaactatcagcagccctcaccgcccgtgtgcggtgccatggcacccaccgaccggttccgtgccgcggtgcaggacgcgaagatcggccccgacaaggacattccgcacgtcacggccgacgtgatcgtcaagtacatcagcaacctgcagatgctcggattgctgtaa

SEQ ID NO: 92 - protein, 1168 aa, Mycobacterium smegmatis
MTIETREDRFNRRIDHLFETDPQFAAARPDEAISAAAADPELRLPAAVKQILAGYADRPALGKRAVEFVTDEEGRTTAKLLPRFDTITYRQLAGRIQAVTNAWHNHPVNAGDRVAILGFTSVDYTTIDIALLELGAVSVPLQTSAPVAQLQPIVAETEPKVIASSVDFLADAVALVESGPAPSRLVVFDYSHEVDDQREAFEAAKGKLAGTGVVVETITDALDRGRSLADAPLYVPDEADPLTLLIYTSGSTGTPKGAMYPESKTATMWQAGSKARWDETLGVMPSITLNFMPMSHVMGRGILCSTLASGGTAYFAARSDLSTFLEDLALVRPTQLNFVPRIWDMLFQEYQSRLDNRRAEGSEDRAEAAVLEEVRTQLLGGRFVSALTGSAPISAEMKSWVEDLLDMHLLEGYGSTEAGAVFIDGQIQRPPVIDYKLVDVPDLGYFATDRPYPRGELLVKSEQMFPGYYKRPEITAEMFDEDGYYRTGDIVAELGPDHLEYLDRRNNVLKLSQGEFVTVSKLEAVFGDSPLVRQIYVYGNSARSYLLAVVVPTEEALSRWDGDELKSRISDSLQDAARAAGLQSYEIPRDFLVETTPFTLENGLLTGIRKLARPKLKAHYGERLEQLYTDLAEGQANELRELRRNGADRPVVETVSRAAVALLGASVTDLRSDAHFTDLGGDSLSALSFSNLLHEIFDVDVPVGVIVSPATDLAGVAAYIEGELRGSKRPTYASVHGRDATEVRARDLALGKFIDAKTLSAAPGLPRSGTEIRTVLLTGATGFLGRYLALEWLERMDLVDGKVICLVRARSDDEARARLDATFDTGDATLLEHYRALAADHLEVIAGDKGEADLGLDHDTWQRLADTVDLIVDPAALVNHVLPYSQMFGPNALGTAELIRIALTTTIKPYVYVSTIGVGQGISPEAFVEDADIREISATRRVDDSYANGYGNSKWAGEVLLREAHDWCGLPVSVFRCDMILADTTYSGQLNLPDMFTRLMLSLVATGIAPGSFYELDADGNRQRAHYDGLPVEFIAEAISTIGSQVTDGFETFHVMNPYDDGIGLDEYVDWLIEAGYPVHRVDDYATWLSRFETALRALPERQRQASLLPLLHNYQQPSPPVCGAMAPTDRFRAAVQDAKIGPDKDIPHVTADVIVKYISNLQMLGLL

SEQ ID NO: 93 - DNA, 1422 bp, Marinobacter hydrocarbonoclasti
atgaaacgtctcggaaccctggacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccagcgccgtggtgcaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 94 - protein, 473 aa, Marinobacter hydrocarbonoclasti
MKRLGTLDASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGAKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPVFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 95 - DNA, 1422 bp, Artificial Sequence
  source: /note=“Description of Artificial Sequence Synthetic polynucleotide”
atgaaacgtctcggaaccctgaacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgtaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgacccagtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 96 - protein, 473 aa, Artificial Sequence
  source: /note=“Description of Artificial Sequence Synthetic polypeptide”
MKRLGTLNASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGVKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTQYTMLLMSPYILQLMSGLGGRMRPFFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 97 - DNA, 1422 bp, Artificial Sequence
  source: /note=“Description of Artificial Sequence Synthetic polynucleotide”
atgaaacgtctcggaaccctgaacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgtaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgaggaaacttccaaaaagtgccctgacccagtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 98 - protein, 473 aa, Artificial Sequence
  source: /note=“Description of Artificial Sequence Synthetic polypeptide”
MKRLGTLNASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGVKTDKEASVPAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLRKLPKSALTQYTMLLMSPYILQLMSGLGGRMRPFFNVTISNVPGPEGTLYYEGARLEAMYPVSLIAHGGALNITCLSYAGSLNFGFTGCRDTLPSMQKLAVYTGEALDELESLILPPKKRARTRK

SEQ ID NO: 99 - DNA, 1422 bp, Artificial Sequence; identical to SEQ ID NO: 95
  source: /note=“Description of Artificial Sequence Synthetic polynucleotide”

SEQ ID NO: 100 - protein, 473 aa, Artificial Sequence; identical to SEQ ID NO: 96
  source: /note=“Description of Artificial Sequence Synthetic polypeptide”

SEQ ID NO: 101 - DNA, 1422 bp, Artificial Sequence
  source: /note=“Description of Artificial Sequence Synthetic polynucleotide”
atgaaacgtctcggaaccctgaacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgtaaaaaccgacaaagaggccagcgtgcccgcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgtgctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgaggaaacttccaaaaagtgccctgacccagtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcatgcagaaactggcggtttataccggtgaagctctggatgagctggaatcgctgattctgccacccaagaagcgcgcccgaacccgcaagtaa

SEQ ID NO: 102 - protein, 473 aa, Artificial Sequence; identical to SEQ ID NO: 98
  source: /note=“Description of Artificial Sequence Synthetic polypeptide”

SEQ ID NO: 103 - DNA, 1422 bp, Artificial Sequence
  source: /note=“Description of Artificial Sequence Synthetic polynucleotide”
atgaaacgtctcggaaccctgaacgcctcctggctggcggttgaatctgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccgacactcagccctgcctcgccccggcggggagcgcgaactgggtattctggtatcccgactgcactctaaccccctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacggtacgcccacaccaacgccgtggtgtaaaaaccgacaaagaggccagcaggcccgcagcggtttcccaggcaatggacgccctgaagctccaggcagacatggcccccaggctgtggcaggccgcgaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttttatgattgcctcgctggccaccgacgaagctgatccgttgaaccgcctgcaacagatcaaaacctcgacccgacgggccaaggagcacctgcagaaacttccaaaaagtgccctgaccgtgtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccggtatcgctaatcgctcacggcggcgccctgaacatcacctgcctgagctatgccggatcgctgaatttcggttttaccggctgtcgggatacgctgccgagcggccagaaactggcggtttataccggtgaagctctggatgagctggaatcgctgattctgccacccaagaagcgcgcccgaacccgcaagtaa

SEQ ID NO: 104 - protein, 473 aa, Artificial Sequence
  source: /note=“Description of Artificial Sequence Synthetic polypeptide”
MKRLGTLNASWLAVESEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNPLDFSRPLWECHVIEGLENNRFALYTKMHHSMIDGISGVRLMQRVLTTDPERCNMPPPWTVRPHQRRGVKTDKEASRPAAVSQAMDALKLQADMAPRLWQAANRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISFMIASLATDEADPLNRLQQIKTSTRRAKEHLQKLPKSALTVYTMLLMSPYILQLMSGLGGRMRPFFNVTISNVPGPEGTLYYEGARLEAMYPVSLIAHGGALNITCLSYAGSLNFGFTGCRDTLPSGQKLAVYTGEALDELESLILPPKKRARTRK

SEQ ID NO: 105 - DNA, 1422 bp, Artificial Sequence
  source: /note=“Description of Artificial Sequence Synthetic polynucleotide”
atgaaacgtctcggatccctggacgcctcctggctggcggttgaaggtgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttggatgattgcctcgctggccaccgacgaagctgatccgttgaaccgcctgcaacagatcaaaacctcgacccgacgggccaaggagcacctgcagaaacttccaaaaacggccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 106 - protein, 473 aa, Artificial Sequence
  source: /note=“Description of Artificial Sequence Synthetic polypeptide”
MKRLGSLDASWLAVEGEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNSLDFSRPLWECHVIEGLENNRFALYTKMHHSMIDGISGVRLMQRVLTTDPERCNMPPPWTRRPHQRRGAKTDKEASVRAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISWMIASLATDEADPLNRLQQIKTSTRRAKEHLQKLPKTALTQYTMLLMSPYILQLMSGLGGRMRPVFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 107 - DNA, 1422 bp, Artificial Sequence
  source: /note=“Description of Artificial Sequence Synthetic polynucleotide”
atgaaacgtctcggatccctggacgcctcctggctggcggttgaaggtgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgatttcgatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggttgtgcaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccaggcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttggatgattgcctcgctggccaccgacgaagctgatccgttgaaccgcctgcaacagatcaaaacctcgacccgacgggccaaggagcacctgcagcaccttccaaaaacggccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 108 - protein, 473 aa, Artificial Sequence
  source: /note=“Description of Artificial Sequence Synthetic polypeptide”
MKRLGSLDASWLAVEGEDTPMHVGTLQIFSLPEGAPETFLRDMVTRMKEAGDVAPPWGYKLAWSGFLGRVIAPAWKVDFDIDLDYHVRHSALPRPGGERELGILVSRLHSNSLDFSRPLWECHVIEGLENNRFALYTKMHHSMIDGISGVRLMQRVLTTDPERCNMPPPWTRRPHQRRGAKTDKEASVRAAVVQAMDALKLQADMAPRLWQAGNRLVHSVRHPEDGLTAPFTGPVSVLNHRVTRQRRFATQHYQLDRLKNLAHASGGSLNDIVLYLCGTALRRFLAEQNNLPDTPLTAGIPVNIRPADDEGTGTQISWMIASLATDEADPLNRLQQIKTSTRRAKEHLQHLPKTALTQYTMLLMSPYILQLMSGLGGRMRPVFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 109 - DNA, 1422 bp, Artificial Sequence
  source: /note=“Description of Artificial Sequence Synthetic polynucleotide”
atgaaacgtctcggatccctggacgcctcctggctggcggttgaaggtgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaactggataaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaggaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcgggtggatgattgcctcgctggccaccgacgaagctgatccgttgaaccgcctgcaacagatcaaaacctcgacccgacgggccaaggagcacctgcagaaacttccaaaaacggccctgacccagtacacccgcctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 110 - protein, 473 aa, Artificial Sequence
  source: /note=“Description of Artificial Sequence Synthetic polypeptide”
MKRLGSLDASWLAVEGEDTPMHVGTLQIFSLPEGAPETFLRDMVTRMKEAGDVAPPWGYKLAWSGFLGRVIAPAWKLDKDIDLDYHVRHSALPRPGGERELGILVSRLHSNSLDFSRPLWECHVIEGLENNRFALYTKMHHSMIDGISGVRLMQRVLTTDPERCNMPPPWTRRPHQRRGAKTDKEASVRAAVSQAMDALKLQADMAPRLWQAGNRLVHSVRHPEDGLTAPFTGPVSVLNHRVTAQRRFATQHYQLDRLRNLAHASGGSLNDIVLYLCGTALRRFLAEQNNLPDTPLTAGIPVNIRPADDEGTGTQIGWMIASLATDEADPLNRLQQIKTSTRRAKEHLQKLPKTALTQYTRLLMSPYILQLMSGLGGRMRPVFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 111 - DNA, 1422 bp, Artificial Sequence
  source: /note=“Description of Artificial Sequence Synthetic polynucleotide”
atgaaacgtctcggatccctggacgcctcctggctggcggttgaaggtgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgggccgacgttttgccacccagcattatcaactggaccggctgaaaaacctggcccatgcttccggcggtgggttgaacgacatcgttctttacctgtgtggcaccgcattgcggcgctttctggctgagcagaacaatctgccagacaccccgctgacggctggtataccggtgaatatccggccggcagacgacgaggtcacgggcacccagatcagttggatgatttgttcgctggccaccgacgaagctgatccgttgaaccgcctgcaacagatcaaaacctcgacccgacgggccaaggagcacctgcagaaacttccaaaaacggccctgacccagtacaccatgctgctgatgtcaccctggattctgcaattgatgtcaggtctcggggggaggatgcgaccagtcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 112 - protein, 473 aa, Artificial Sequence
  source: /note=“Description of Artificial Sequence Synthetic polypeptide”
MKRLGSLDASWLAVEGEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNSLDFSRPLWECHVIEGLENNRFALYTKMHHSMIDGISGVRLMQRVLTTDPERCNMPPPWTRRPHQRRGAKTDKEASVRAAVSQAMDALKLQADMAPRLWQAGNRLVHSVRHPEDGLTAPFTGPVSVLNHRVTAGRRFATQHYQLDRLKNLAHASGGGLNDIVLYLCGTALRRFLAEQNNLPDTPLTAGIPVNIRPADDEVTGTQISWMICSLATDEADPLNRLQQIKTSTRRAKEHLQKLPKTALTQYTMLLMSPWILQLMSGLGGRMRPVFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 113 - DNA, 1422 bp, Artificial Sequence
  source: /note=“Description of Artificial Sequence Synthetic polynucleotide”
atgaaacgtctcggaaccctggacgcctcctggctggcggttgaaggtgaagacaccccgatgcatgtgggtacgcttcagattttctcactgccggaaggcgcaccagaaaccttcctgcgtgacatggtcactcgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagaggggcctcaccaccgatcccgaacgctgcaatatgtcaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcacccagatcagttggatgattgcctcgctggccaccgacgaagctgatccgttgaaccgcctgcaacagatcaaaacctcgacccgacgggccaaggagcacctggcgaaacttccaaaaacggccctgacccagtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 114 - protein, 473 aa, Artificial Sequence
  source: /note=“Description of Artificial Sequence Synthetic polypeptide”
MKRLGTLDASWLAVEGEDTPMHVGTLQIFSLPEGAPETFLRDMVTRMKEAGDVAPPWGYKLAWSGFLGRVIAPAWKVDKDIDLDYHVRHSALPRPGGERELGILVSRLHSNSLDFSRPLWECHVIEGLENNRFALYTKMHHSMIDGISGVRLMQRGLTTDPERCNMSPPWTRRPHQRRGAKTDKEASVRAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGTQISWMIASLATDEADPLNRLQQIKTSTRRAKEHLAKLPKTALTQYTMLLMSPYILQLMSGLGGRMRPFFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 115 - DNA, 1422 bp, Artificial Sequence
  source: /note=“Description of Artificial Sequence Synthetic polynucleotide”
atgaaacgtctcggaaccctggacgcctcctggctggcggttgaaggtgaagacaccccgatgcatgtgggtacgcttcagattttctcactgccggaaggcgcaccagaaaccttctcgcgtgacatggtcactcgaatgaaagaggccggcgatgtggcaccaccctggggatacaaactggcctggtctggtttcctcgggcgcgtgatcgccccggcctggaaagtcgcgaaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcatcagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcagacgacgagggtacgggcagtcagatcagttggatgattgcctcgctggccaccgacgaagctgatccgttgaaccgcctgcaacagatcaaaacctcgacccgacgggccaaggagcacctggcgaaacttccaaaaacggccctgacccagtacaccatgctgctgatgtcaccctacattctgcaattgatgtcaggtctcggggggaggatgcgaccattcttcaacgtgaccatttccaacgtgcccggcccggaaggcacgctgtattatgaaggagcccggcttgaggccatgtatccgttgtcgctaatcgctcacggcggcgccctgaacgtgacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 116 - protein, 473 aa, Artificial Sequence
  source: /note=“Description of Artificial Sequence Synthetic polypeptide”
MKRLGTLDASWLAVEGEDTPMHVGTLQIFSLPEGAPETFSRDMVTRMKEAGDVAPPWGYKLAWSGFLGRVIAPAWKVAKDIDLDYHVRHSALPRPGGERELGILVSRLHSNSLDFSRPLWECHVIEGLENNRFALYTKMHHSMIDGISGVRLMQRVLTTDPERCNMPPPWTRRPHQRRGAKTDKEASVRAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPADDEGTGSQISWMIASLATDEADPLNRLQQIKTSTRRAKEHLAKLPKTALTQYTMLLMSPYILQLMSGLGGRMRPFFNVTISNVPGPEGTLYYEGARLEAMYPLSLIAHGGALNVTCLSYAGSLNFGFTGCRDTLPGMQKLAVYTGEALDELESLILPPKKRARTRK

SEQ ID NO: 117 - DNA, 1422 bp, Artificial Sequence
  source: /note=“Description of Artificial Sequence Synthetic polynucleotide”
atgaaacgtctcggatccctggacgcctcctggctggcggttgaaggtgaagacaccccgatgcatgtgggttggcttcagattttctcactgccggaaggcgcaccagaaaccttcctgcgtgacatggtcttccgaatgaaagaggccggcgatgtggcaccaccctggggatacaaactggcctggtctggtttcctcgggcgcgtgatcgccccggcctggaaagtcgataaggatatcgatctggattatcacgtccggcactcagccctgcctcgccccggcggggagcgcgaactgggtattctggtatcccgactgcactctaacagtctggatttttcccgccctctttgggaatgccacgttattgaaggcctggagaataaccgttttgccctttacaccaaaatgcaccactcgatgattgacggcttgagcggcgtgcgactgatgcagagggtgctcaccaccgatcccgaacgctgcaatatgccaccgccctggacgcgccgcccacaccagcgccgtggtgcaaaaaccgacaaagaggccagcgtgcgggcagcggtttcccaggcaatggacgccctgaagctccaggcagacatggcccccaggctgtggcaggccggcaatcgcctggtgcattcggttcgacacccggaagacggactgaccgcgcccttcactggaccggtttcggtgctcaatcaccgggttaccgcgcagcgacgttttgccacccagcattatcaactggaccggctgaaaaacctggcccatgcttccggcggttccttgaacgacatcgttctttacctgtgtggcaccgcattgcggcgctttctggctgagcagaacaatctgccagacaccccgctgacggctggtataccggtgaatatccggccggcaaacgacgagggtacgggcacccagatcagttggatgattgcctcgctggccaccgacgaagctgatccgttgaaccgcctgcaacagatcaaaacctcgacccgacgggccaaggagcacctgcagaaacttccaaaaacggccctgacccagtacaccatgctgctgatgtcaccctacattctgcaattgatgtcaggtctcggggggaggatgcgaccagtcttcaacgtgaccatttccaacgtgcccggcccggaaggcacgctgtattatgaaggagcccggcttgaggccatgtatccgttgtcgctaatcgctcacggcggcgccctgaacatcacctgcctgagctatgccggatcgctgaatttcggttttaccggctgtcgggatacgctgccggggatgcagaaactggcggtttataccggtgaagctctggatgagctggaatcgctgattctgccacccaagaagcgcgcccgaacccgcaagtaa

SEQ ID NO: 118 - protein, 473 aa, Artificial Sequence
  source: /note=“Description of Artificial Sequence Synthetic polypeptide”
MKRLGSLDASWLAVEGEDTPMHVGWLQIFSLPEGAPETFLRDMVFRMKEAGDVAPPWGYKLAWSGFLGRVIAPAWKVDKDIDLDYHVRHSALPRPGGERELGILVSRLHSNSLDFSRPLWECHVIEGLENNRFALYTKMHHSMIDGLSGVRLMQRVLTTDPERCNMPPPWTRRPHQRRGAKTDKEASVRAAVSQAMDALKLQADMAPRLWQAGNRLVHSVRHPEDGLTAPFTGPVSVLNHRVTAQRRFATQHYQLDRLKNLAHASGGSLNDIVLYLCGTALRRFLAEQNNLPDTPLTAGIPVNIRPANDEGTGTQISWMIASLATDEADPLNRLQQIKTSTRRAKEHLQKLPKTALTQYTMLLMSPYILQLMSGLGGRMRPVFNVTISNVPGPEGTLYYEGARLEAMYPLSLIAHGGALNITCLSYAGSLNFGFTGCRDTLPGMQKLAVYTGEALDELESLILPPKKRARTRK

SEQ ID NO: 119 - protein, 455 aa, Marinobacter hydrocarbonoclasticus
MTPLNPTDQLFLWLEKRQQPMHVGGLQLFSFPEGAPDDYVAQLADQLRQKTEVTAPFNQRLSYRLGQPVWVEDEHLDLEHHFRFEALPTPGRIRELLSFVSAEHSHLMDRERPMWEVHLIEGLKDRQFALYTKVHHSLVDGVSAMRMATRMLSENPDEHGMPPIWDLPCLSRDRGESDGHSLWRSVTHLLGLSDRQLGTIPTVAKELLKTINQARKDPAYDSIFHAPRCMLNQKITGSRRFAAQSWCLKRIRAVCEAYGTTVNDVVTAMCAAALRTYLMNQDALPEKPLVAFVPVSLRRDDSSGGNQVGVILASLHTDVQDAGERLLKIHHGMEEAKQRYRHMSPEEIVNYTALTLAPAAFHLLTGLAPKWQTFNVVISNVPGPSRPLYWNGAKLEGMYPVSIDMDRLALNMTLTSYNDQVEFGLIGCRRTLPSLQRMLDYLEQGLAELELNAGL

SEQ ID NO: 120 - DNA, 1000 bp, Marinobacter hydrocarbonoclasticus
atgacgcccctgaatcccactgaccagctctttctctggctggaaaaacgccagcagcccatgcatgtgggcggcctccagctgttttccttccccgaaggcgcgccggacgactatgtcgcgcagctggcagaccagcttcggcagaagacggaggtgaccgccccctttaaccagcgcctgagctatcgcctgggccagccggtatgggtggaggatgagcacctggaccttgagcatcatttccgcttcgaggcgctgcccacacccgggcgtattcgggagctgctgtcgttcgtatcggcggagcattcgcacctgatggaccgggagcgccccatgtgggaggtgcacctgatcgagggcctgaaagaccggcagtttgcgctctacaccaaggttcaccattccctggtggacggtgtctcggccatgcgcatggccacccggatgctgagtgaaaacccggacgaacacggcatgccgccaatctgggatctgccttgcctgtcacgggataggggtgagtcggacggacactccctctggcgcagtgtcacccatttgctggggctttcggaccgccagctcggcaccattcccactgtggcaaaggagctactgaaaaccatcaatcaggcccggaaggatccggcctacgactccattttccatgccccgcgctgcatgctgaaccagaaaatcaccggttcccgtcgattcgccgctcagtcctggtgcctgaaacggattcgcgccgtatgcgaggcctacggcaccacggtcaacgatgtcgtgactgccatgtgcgcagcggctctgcgtacctatctgatgaatcaggatgccttgccggagaaaccactggtggcctttgtgccggtgtcgctacgccgggacgacagctccggcggcaaccaggtaggcgtcatcctggcgagccttcacaccgatgtgcaggacgccggcgaacgactgttaaaaattcaccacggcatgg

SEQ ID NO: 121 - DNA, 234 bp, Marinobacter aquaeolei
atgagtacagttgaagagcgcgttaagaagattgtttgtgagcagttgggcgtgaaagagtccgaagttcagaacacatcttcttttgtagaggatcttggcgctgactcactggacactgttgagctggttatggccctggaagaggaattcgagacagagattcctgacgaagaggccgaaaagctgggcaccgttcaggacgcgatcgactacattgtcgcgcacacctga

SEQ ID NO: 122 - protein, 77 aa, Marinobacter aquaeolei
MSTVEERVKKIVCEQLGVKESEVQNTSSFVEDLGADSLDTVELVMALEEEFETEIPDEEAEKLGTVQDAIDYIVAHT

SEQ ID NO: 123 - DNA, 234 bp, Marinobacter hydrocarbonoclasticus
atgagtacagttgaagagcgcgttaagaagattgtttgtgagcagttgggcgtgaaagagtccgaagttcagaacacatcttcttttgtagaggatcttggcgctgactcactggacactgttgagctggttatggccctggaagaggaattcgagaccgagattcctgacgaagaggccgaaaagctgggcaccgttcaggacgcgatcgactacattgtcgcgcacacctga

SEQ ID NO: 124 - protein, 77 aa, Marinobacter hydrocarbonoclasticus; identical to SEQ ID NO: 122

(19) United States
(12) Patent Application Publication
(10) Pub. No.: US 2016/0002681 A1
(43) Pub. Date: Jan. 7, 2016

(54) ACP-MEDIATED PRODUCTION OF FATTY ACID DERIVATIVES

(71) Applicant: REG LIFE SCIENCES, LLC, South San Francisco, CA (US)

(72) Inventors: David SIMPSON, South San Francisco, CA (US); Bernardo DA COSTA, South San Francisco, CA (US); Mathew RUDE, South San Francisco, CA (US); Na TRINH, South San Francisco, CA (US); Emanuela POPOVA, South San Francisco, CA (US); Sankaranarayanan VENKITESWARAN, South San Francisco, CA (US); Noah HELMAN, South San Francisco, CA (US); Kevin HOLDEN, South San Francisco, CA (US)

(73) Assignee: REG LIFE SCIENCES, LLC, South San Francisco, CA (US)

(21) Appl. No.: 14/760,204

(22) PCT Filed: Dec. 11, 2013
(86) PCT No.: PCT/US2013/074427

§ 371 Date: Jul. 9, 2015

Related U.S. Application Data
(60) Provisional application No. 61/736,428, filed on Dec. 12, 2012.

(51) Int. Cl. C12P 7/64; C12N 9/16; C12N 9/12; C12P 7/04; C12N 9/02

(52) U.S. Cl. CPC C12P 7/6436; C12P 7/6409; C12P 7/04; C12N 9/001; C12N 9/1288; C12N 9/16; C12Y 103/01009; C12Y 207/08; C12Y 301/02

(57) ABSTRACT

The disclosure relates to recombinant microorganisms that exhibit an increased expression of an acyl carrier protein (ACP) resulting in production of fatty acid derivatives. The disclosure further relates to methods of using the recombinant microorganisms in fermentation cultures in order to produce fatty acid derivatives and related compositions.

This application claims the benefit of U.S. Provisional Application No. 61/736,428, filed Dec. 12, 2012, the contents of which are hereby incorporated by reference in their entirety.

### SEQUENCE LISTING

The instant application contains a Sequence Listing which has been submitted electronically in ASCII format and is hereby incorporated by reference in its entirety. Said ASCII copy, created on Nov. 1, 2013, is named LS00045PCT_SL.txt and is 232,659 bytes in size.

### FIELD

The disclosure relates to recombinant microorganisms that exhibit an increased expression of an acyl carrier protein (ACP) resulting in production of fatty acid derivatives. The disclosure further relates to methods of using the recombinant microorganisms in fermentation cultures in order to produce fatty acid derivatives and related compositions.

### BACKGROUND

Fatty acid derivatives such as fatty aldehydes, fatty alcohols, hydrocarbons (e.g., alkanes and olefins), fatty esters (e.g., waxes, fatty acid esters, fatty esters) and ketones provide the building blocks for important categories of industrial chemicals and fuels. These compounds have numerous industrial applications including as surfactants, lubricants, solvents, emulsifiers, emollients, thickeners, flavors, fragrances, and fuels. For example, biodiesel, an alternative fuel, is made primarily of esters such as fatty acid methyl esters (FAME), fatty acid ethyl esters (FAEE), and the like. Some low molecular weight esters are volatile with a pleasant odor and are used for the production of fragrances and flavoring agents. In addition, fatty esters are used as solvents for lacquers, paints, and varnishes; as softening agents in resins and plastics, as plasticizers, as flame retardants, as additives in gasoline and oil and in the manufacture of polymers, films, textiles, dyes, and pharmaceuticals.

In nature, most fatty alcohols are found as waxes, which are esters with fatty acids and fatty alcohols produced by bacteria, plants and animals. In the industrial setting, fatty alcohols have many commercial uses. The shorter chain fatty alcohols are used in the cosmetic and food industries as emulsifiers, emollients, and thickeners. Due to their amphiphilic nature, fatty alcohols behave as nonionic surfactants, which are useful in personal care and household products, such as cosmetics and detergents. In addition, fatty alcohols are used in waxes, gums, resins, pharmaceutical salves and lotions, lubricating oil additives, textile antistatic and finishing agents, plasticizers, industrial solvents, and solvents for fats. Fatty alcohols are aliphatic alcohols with a chain length of 8 to 22 carbon atoms. Fatty alcohols usually have an even number of carbon atoms and a single alcohol group (OH) attached to the terminal carbon, wherein some are unsaturated and some are branched. Fatty alcohols are also widely used in industrial chemistry.

Fatty aldehydes can be used to produce industrial specialty chemicals. For example, aldehydes are commonly used to produce polymers, resins, dyes, flavorings, plasticizers, perfumes, and pharmaceuticals. Aldehydes can also be used as solvents, preservatives, and disinfectants. Certain natural and synthetic compounds, such as vitamins and hormones, are aldehydes, and many sugars contain aldehyde groups. Fatty aldehydes can be converted to fatty alcohols by chemical or enzymatic reduction.

Historically, industrial chemicals and fuels have been produced from petrochemicals. The petrochemical raw materials are fatty acids, fatty esters, fatty alcohols, fatty aldehydes, ketones, hydrocarbons and the like. Due to the inherent challenges posed by exploring, extracting, transporting and refining petroleum for use in industrial chemicals and fuel products, there is a need for a an alternate way for producing raw materials that is more cost effective and environmentally friendly. One such alternative way is the production of biologically-derived chemicals and fuels from fermentable carbon sources. However, in order for biologically-derived chemicals and fuels to be produced from fermentable sugars or biomass in a commercially viable manner, existing processes must be continuously optimized for efficient conversion and recovery of product. Although there have been notable successes in the industry, there still remains a need for further improvements in the relevant processes in order for biologically-derived chemicals and fuels to become more widely available alternatives. Areas for improvement include the efficiency of the production process and product yield. The current disclosure addresses this need.

### SUMMARY

The present disclosure provides novel recombinant host cells with vector and strain modifications effective to result in an increase in the amount of acyl carrier protein (ACP) available for fatty acid biosynthesis in order to produce fatty acid derivative compositions. The disclosure also provides methods of making fatty acid derivative compositions by culturing the recombinant host cells and collecting the fatty acid derivative compositions from the culture medium. Examples of fatty acid derivative compositions include, but are not limited to, compositions that encompass fatty acids, fatty esters, fatty alcohols, fatty aldehydes, ketones, alkanes, alkenes, olefins, and/or combinations thereof.

One aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition. In one particular aspect, the recombinant host cell produces the fatty acid derivative composition with a higher titer, a higher yield and/or a higher productivity when cultured in a medium containing a carbon source under conditions effective to overexpress the polynucleotide sequences as compared to a corresponding wild type host cell propagated under the same conditions as the recombinant host cell. Thus, the recombinant host cell produces a fatty acid derivative compositions that includes the fatty acid derivative at a higher titer, a higher yield and/or a higher productivity then the corresponding wild type host cell. The fatty acid derivative includes, but is not limited to, a fatty acid, a fatty alcohol, a fatty ester, a fatty aldehyde, an alkane, an alkene, an olefin, and/or a ketone. In one embodiment, the recombinant host cell includes a polynucleotide sequence encoding a heterologous acyl carrier protein (ACP), and a polynucleotide sequence encoding a heterologous fatty acid derivative biosynthetic protein that has thioesterase activity, wherein the recombinant host cell produces a fatty acid derivative composition that includes a fatty acid. In another embodiment, the recombinant host cell includes a polynucleotide sequence encoding a heterologous acyl carrier protein (ACP), a polynucleotide sequence encoding a heterologous fatty acid derivative biosynthetic protein that has thioesterase activity, and a protein that has carboxylic acid reductase (CAR) activity, wherein the recombinant host cell produces a fatty acid derivative composition that includes a fatty alcohol. In still another embodiment, the recombinant host cell includes a polynucleotide sequence encoding a heterologous acyl carrier protein (ACP), and a polynucleotide sequence encoding a heterologous fatty acid derivative biosynthetic protein that has acyl-ACP reductase (AAR) activity, wherein the recombinant host cell produces a fatty acid derivative composition that includes a fatty alcohol. In yet another embodiment, the recombinant host cell includes a polynucleotide sequence encoding a heterologous acyl carrier protein (ACP), and a polynucleotide sequence encoding a heterologous fatty acid derivative biosynthetic protein that has ester synthase activity, wherein the recombinant host cell produces a fatty acid derivative composition that includes a fatty ester.

Another aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition at a higher titer and that is at least about 10% to at least about 90% greater compared to the corresponding wild type host cell. The fatty acid derivative composition includes, but is not limited to, a composition with a fatty acid, a fatty alcohol, a fatty ester, a fatty aldehyde, an alkane, an alkene, an olefin, and/or a ketone.

Another aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition at a yield that is at least about 5% to at least about 80% greater compared to the corresponding wild type host cell. The fatty acid derivative composition includes, but is not limited to, a composition with a fatty acid, a fatty alcohol, a fatty ester, a fatty aldehyde, an alkane, an alkene, an olefin, and/or a ketone.

Another aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition at a titer of from about 100 mg/L to about 300 g/L; and/or a titer of from about 1 g/L to about 250 g/L; and/or a titer of at least about 30 g/L or about 35 g/L or about 40 g/L or about 45 g/L or about 50 g/L or about 55 g/L or about 60 g/L or about 65 g/L or about 70 g/L or about 75 g/L or about 80 g/L or about 85 g/L or about 90 g/L or about 95 g/L or about 100 g/L or about 150 g/L or about 200 g/L. In addition, the fatty acid derivative composition is produced at a productivity of from about 0.7 mg/L/hr to about 2.5 g/L/hr.

Another aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition. In one embodiment, the ACP is a cyanobacterial acyl carrier protein (cACP). In another embodiment, the ACP is a Marinobacter aquaeolei VT8 acyl carrier protein (mACP). In another embodiment, the ACP is an Escherichia coli acyl carrier protein (ecACP).

Another aspect of the disclosure provides a recombinant host cell that includes a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition. In one particular aspect, the recombinant host cell further expresses a sfp gene encoding a 4′-phosphopantetheinyl transferase (PPTase) protein. In one embodiment, the sfp gene is a B. subtilis sfp gene that is heterologous to the recombinant cell. In another embodiment, the recombinant cell has a native 4′-phosphopantetheinyl transferase protein. In yet another embodiment, the recombinant host cell produces a fatty acid derivative composition extracellularly. In still another embodiment, the recombinant host cell produces a fatty acid derivative composition intercellularly.

The disclosure further contemplates a cell culture that includes a recombinant host cell expressing a polynucleotide sequence encoding a heterologous acyl carrier protein (ACP), and a polynucleotide sequence encoding a heterologous fatty acid derivative biosynthetic protein, wherein the recombinant host cell produces a fatty acid derivative composition. In one embodiment, the fatty acid derivative composition (e.g., fatty acid, fatty alcohol, fatty ester) is found in a culture medium. The fatty acid derivative of the composition is a C6, C8, C10, C12, C13, C14, C15, C16, C17, and/or C18 fatty acid derivative. In one embodiment, the fatty acid derivative of the composition is an unsaturated fatty acid derivative such as a C10:1, C12:1, C14:1, C16:1, and/or C18:1 unsaturated fatty acid derivative. In another embodiment, the fatty acid derivative of the composition is a saturated fatty acid derivative. In one particular embodiment, the fatty acid derivative composition includes a fatty acid derivative that has a double bond between the 7th and 8th carbon from the reduced end of the fatty acid, the fatty ester, or the fatty alcohol. In yet another embodiment, the fatty acid derivative composition includes a branched chain fatty acid derivative. In still another embodiment, the fatty acid derivative composition includes a fatty acid derivative that has a fraction of modern carbon of about 1.003 to about 1.5; and/or a δ13C of from about −10.9 to about −15.4.

Another aspect of the present disclosure provides a method of making a fatty acid derivative composition. The method includes culturing a recombinant host cell as described above (supra) in the presence of a carbon source in order to produce a fatty acid derivative composition, and collecting the fatty acid derivative composition from the culture medium, wherein the yield, titer and/or productivity of the fatty acid derivative composition is at least about 10% greater than the yield, titer and/or productivity of a fatty acid derivative composition produced by a corresponding wild type host cell cultured under the same conditions. The method optionally includes isolating the produced fatty acid derivative composition from the recombinant host cell. In one particular embodiment, the fatty acid derivative composition is found in the culture medium. The fatty acid derivative composition includes, but is not limited to, a fatty acid, a fatty alcohol, a fatty ester, a fatty aldehyde, an alkane, an alkene, an olefin, and a ketone. In one embodiment, the fatty acid derivative composition includes a fatty acid or a fatty alcohol or a fatty ester or a fatty aldehyde or an alkane or an alkene or an olefin or a ketone. In another embodiment, the fatty acid derivative composition is a combination of any one or more fatty acid derivatives, including, but not limited to, a fatty acid, a fatty alcohol, a fatty ester, a fatty aldehyde, an alkane, an alkene, an olefin (e.g., an internal olefin or a terminal olefin), and a ketone. The fatty acid derivative composition can include saturated and/or unsaturated fatty acid derivatives. In one embodiment, the method produces fatty acid derivative compositions that include a C6, C8, C10, C12, C13, C14, C15, C16, C17, or C18 fatty acid derivative. In one particular embodiment, the method produces fatty acid derivative compositions that include a C10:1, C12:1, C14:1, C16:1, or C18:1 unsaturated fatty acid derivative. In another particular embodiment the fatty acid derivative composition includes a fatty acid. In another particular embodiment the fatty acid derivative composition includes a fatty alcohol. In another particular embodiment the fatty acid derivative composition includes a fatty ester. In another particular embodiment the fatty acid derivative composition includes a fatty aldehyde. In another particular embodiment the fatty acid derivative composition includes an alkane. In another particular embodiment the fatty acid derivative composition includes an alkene. In another particular embodiment the fatty acid derivative composition includes an olefin such as an internal and/or a terminal olefin. In another particular embodiment the fatty acid derivative composition includes a ketone. In another embodiment, the fatty acid derivative composition includes a branched chain fatty acid derivative. In yet another embodiment, the fatty acid derivative composition includes fatty acid derivative that has a fraction of modern carbon of about 1.003 to about 1.5; and/or a δ13C of from about −10.9 to about −15.4. In still another particular embodiment, the fatty acid derivative composition includes a fatty acid derivative having a double bond between the 7th and 8th carbon from the reduced end of the fatty acid, the fatty ester, and/or the fatty alcohol.

The present disclosure further contemplates a fatty acid derivative composition as produced by the method as described above (supra) that includes a fatty acid derivative that has a double bond between the 7th and 8th carbon from the reduced end of the fatty acid, the fatty ester, and/or the fatty alcohol. This fatty acid derivative composition is produced by a recombinant host cell that includes a polynucleotide sequence encoding a heterologous acyl carrier protein (ACP), and a polynucleotide sequence encoding a heterologous fatty acid derivative biosynthetic protein as described herein (supra).

The present disclosure provides novel recombinant host cells, related methods and processes which produce fatty acid derivative compositions at a higher titer, higher yield and/or higher productivity than a corresponding wild type host cell propagated under the same conditions as the recombinant host cells. Particularly, one aspect of the disclosure provides recombinant host cells that include or express a polynucleotide sequence that encodes a heterologous acyl carrier protein (ACP) and a polynucleotide sequence that encodes a heterologous fatty acid derivative biosynthetic protein, wherein the recombinant host cells produce a fatty acid derivative or a fatty acid derivative composition. In another aspect, the disclosure provides recombinant host cells that include or express a polynucleotide sequence encoding a heterologous acyl carrier protein (ACP); a polynucleotide sequence encoding a heterologous phosphopantetheinyltransferase (PPTase) protein; and a polynucleotide sequence encoding a heterologous fatty acid derivative biosynthetic protein, wherein the recombinant host cells produce a fatty acid derivative composition. In one embodiment, the ACP is a cyanobacterial acyl carrier protein (cACP). In another embodiment, the ACP is a Marinobacter aquaeolei VT8 acyl carrier protein (mACP). In yet another embodiment, the ACP is an E. coli acyl carrier protein (ecACP). In yet another embodiment, the phosphopantetheinyltransferase (PPTase) protein is a 4′-phosphopantetheinyl transferase protein encoded by the sfp gene. The fatty acid derivative biosynthetic protein includes, but is not limited to, a protein that has thioesterase activity; a protein that has carboxylic acid reductase (CAR) activity; a protein that has acyl ACP reductase (AAR) activity; and/or a protein that has ester synthase activity. In one particular aspect, the recombinant host cells produce a fatty acid derivative composition with a higher titer, higher yield and/or higher productivity when cultured in a medium containing a carbon source under conditions effective to overexpress the polynucleotide sequences, when compared to corresponding wild type host cells propagated under the same conditions as the recombinant host cells. The fatty acid derivative compositions that are produced by the recombinant host cells include, but are not limited to, fatty acids, fatty esters, fatty alcohols, fatty aldehydes, ketones, alkanes, alkenes, olefins, and/or combinations thereof.

Another aspect of the present disclosure provides recombinant host cells that include or express a polynucleotide sequence that encodes a heterologous acyl carrier protein (ACP) and a polynucleotide sequence that encodes a heterologous fatty acid derivative biosynthetic protein that has thioesterase activity. In one embodiment, the fatty acid derivative biosynthetic protein is a thioesterase protein. In another embodiment, the recombinant host cells further include or express a polynucleotide sequence encoding a heterologous phosphopantetheinyltransferase (PPTase) protein. Herein, the fatty acid derivative compositions that are produced by these recombinant host cells are fatty acids. In another embodiment, the recombinant host cells further include or express a protein with carboxylic acid reductase (CAR) activity. In yet another embodiment, the recombinant host cells further include or express a carboxylic acid reductase (CAR) protein. The fatty acid derivative compositions that are produced by these recombinant host cells are fatty alcohols and/or fatty aldehydes.

Another aspect of the present disclosure provides recombinant host cells that include or express a polynucleotide sequence that encodes a heterologous acyl carrier protein (ACP) and a polynucleotide sequence that encodes a heterologous fatty acid derivative biosynthetic protein that has carboxylic acid reductase (CAR) activity. In one embodiment, the recombinant host cells include or express a carboxylic acid reductase (CAR) protein. In another embodiment, the recombinant host cells further include or express a polynucleotide sequence encoding a heterologous phosphopantetheinyltransferase (PPTase) protein. The fatty acid derivative compositions that are produced by these recombinant host cells are fatty alcohols and/or fatty aldehydes.

Another aspect of the present disclosure provides host cell that include or express a polynucleotide sequence that encodes a heterologous acyl carrier protein (ACP) and a polynucleotide sequence that encodes a heterologous fatty acid derivative biosynthetic protein that has acyl-ACP reductase (AAR) activity. In one embodiment, the recombinant host cells include or express an acyl-ACP reductase (AAR) protein. In another embodiment, the recombinant host cell further include or express a polynucleotide sequence encoding a heterologous phosphopantetheinyltransferase (PPTase) protein. The fatty acid derivative compositions that are produced by these recombinant host cells are fatty alcohols and/or fatty aldehydes.

The disclosure also encompasses cell cultures including the novel recombinant host cells and methods of using the cell cultures. The disclosure further provides methods of making compositions including fatty acid derivatives by culturing the recombinant host cells of the disclosure, compositions made by such methods, and other features apparent upon further review.

In one aspect, the disclosure provides a cultured recombinant host cell including a polynucleotide sequence encoding a heterologous ACP protein, and a polynucleotide sequence encoding a fatty acid derivative biosynthetic polypeptide, wherein the cultured recombinant host cell produces a fatty acid derivative composition with a higher titer, higher yield or higher productivity of fatty acid derivatives when cultured in a medium containing a carbon source under conditions effective to overexpress the polynucleotides, as compared to the expression level in a corresponding wild type host cell propagated under the same conditions as the recombinant host cell. The fatty acid derivative composition includes a fatty acid derivative such as a fatty acid, a fatty aldehyde, a fatty alcohol, a fatty ester, an alkane, an alkene, an olefin, and/or a ketone. The ACP may be a cyanobacterial acyl carrier protein (cACP), a Marinobacter hydrocarbonoclasticus acyl carrier protein (mACP), or an E. coli acyl carrier protein (ecACP). The recombinant and cultured host cell may further comprise an sfp gene, wherein the sfp gene may be a B. subtilis sfp gene, encoding a modified 4′-phosphopantetheinyl transferase (PPTase) protein, which transfers the 4′-phosphopantetheinyl moiety of coenzyme A (CoA) to a serine residue. These and other embodiments will readily occur to those of ordinary skill in the art in view of the present disclosure provided herein.

### BRIEF DESCRIPTION OF THE DRAWINGS

The present disclosure is best understood when read in conjunction with the accompanying figures, which serve to illustrate the preferred embodiments. It is understood, however, that the disclosure is not limited to the specific embodiments disclosed in the figures.

FIG. 1 is a schematic overview of an exemplary biosynthetic pathway for use in the production of acyl-CoA as a precursor to fatty acid derivative production in a recombinant host cell. The cycle is initiated by condensation of malonyl-ACP and acetyl-CoA.

FIG. 2 depicts another schematic overview of an exemplary fatty acid biosynthetic cycle that begins with the condensation of malonyl-ACP and acyl-ACP and ends with acyl-ACP.

FIG. 3 illustrates the structure and function of the acetyl-CoA carboxylase enzyme complex (encoded by the accABCD gene).

FIG. 4 presents a schematic overview of an exemplary biosynthetic pathway for the production of fatty alcohols starting with acyl-ACP.

FIG. 5 presents an overview of two exemplary biosynthetic pathways for the production of fatty esters starting with acyl-ACP.

FIG. 6 presents another overview of exemplary biosynthetic pathways for the production of hydrocarbons (olefins and alkanes) starting with acyl-ACP.

FIG. 7 illustrates a fatty acid production in E. coli DV2 cells by expressing a leaderless E. coli thioesterase (encoded by the ‘tesA gene) and coexpressing a cyanobacterial acyl carrier protein (cACP) and B. subtilis sfp in a standard micro titer plate fermentation experiment.

FIG. 8 illustrates a fatty alcohol production in E. coli DV2 cells by expressing Synechococcus elongatus acyl-ACP reductase (AAR) and coexpressing various cyanobacterial acyl carrier proteins (ACPs) from Table 2.

FIG. 9 shows the results of a 96 well plate fermentation of strains containing the pEP.100 plasmid. The stEP604 strains produced a large titer improvement (3 fold) over the control strain sven038. The same plasmid in the BD64 strain background resulted in slightly lower titers than the control KEV075 strain.

FIG. 10 shows the results of a 5 liter tank fermentation of the stEP604 strain. The stEP604 strain consistently produced a higher titer relative to the control (sven38) throughout the run.

FIG. 11 shows the results of a plate fermentation of strains engineered to overexpress mACP. All strains were derived from the GLPH-077 host strain and were compared with and without ACP overexpression.

FIG. 12 illustrates the effect of overexpression of ecACP on total titer (g/L of total Fatty Acid Species (FAS)) and percent (%) omega-hydroxy (β-OH) ester production in strains that contain pKEV022 or pSHU018.

FIG. 13 shows the FAS titer (g/L) during a 5 liter bioreactor fermentation of strains that overexpress mACP or ecACP (i.e., 24 to 72 hours). The results illustrate that pSHU18 with ecACP outperformed the other ester synthase variants in terms of total FAS production.

FIG. 14 illustrates the percentage of omega-hydroxy (β-OH) FAME produced by various strains when cultured in 5 liter bioreactors. The pSHU18 strain that overexpressed ecACP produced approximately 68% β-OH FAME.

FIG. 15 shows the percent (%) yield on glucose during 5 liter bioreactor fermentation runs with data comparing yield on glucose (i.e., 24 to 72 hours). The pSHU18 strain that overexpressed ecACP clearly exhibited a higher yield than other strains tested in this study.

FIG. 16 illustrates mg/L of alkane production in strain iDJ containing the plasmid pDS171S (see third column to the right). The expression of Nostoc 73102 acp+sfp demonstrated improved alkane production. The controls (no acp/sfp) were pLS9-185 (see first and second column).

### DETAILED DESCRIPTION

General Overview

One way of eliminating our dependency on petroleum and petrochemicals is to produce fatty acid derivatives through environmentally friendly microorganisms that serve as miniature production hosts. Such cellular hosts (i.e., production host cells or production strains) have been engineered to produce fatty acid derivatives from renewable sources such as renewable feedstock (e.g., fermentable sugars, carbohydrates, biomass, cellulose, glycerol, CO, CO₂, etc.). These fatty acid derivatives are the raw materials or building blocks for most industrial products including industrial specialty chemicals and fuels. Biologically derived fatty acid derivatives that provide the basis for biologically derived chemicals and fuels offer distinct advantages over chemicals and fuels that are made from petroleum. First and foremost, they offer a cleaner alternative by protecting the environment and conserving natural resources. The population is estimated to reach 9 billion by 2050 and natural oil reserves are steadily declining. Secondly, the manufacture of biologically derived chemicals and fuels reduces global warming risks by allowing for a production method that is gentler to the planet and more sustainable. Thirdly, the manufacture of biologically derived chemicals and fuels is in alignment with rising energy costs because the manufacturing processes use renewable carbon sources (e.g., carbohydrates, CO₂, biomass, glycerol) which are far less costly than the harvesting and fat-splitting processes of petroleum. For example, the abundance of a high content of carbohydrates in lignocellulosic biomass makes it an attractive feedstock for enzymatic reactions. Similar low cost and abundant renewable feedstocks include CO₂ and glycerol which are the bi-products of other industrial processes.

The biologically derived chemicals and fuels that are contemplated herein are made from fatty acids, fatty esters, fatty alcohols, fatty aldehydes, hydrocarbons (e.g., alkanes, alkenes and/or olefins) and/or ketones. As such, they can be produced from fermentable sugars, carbohydrates, biomass, CO₂, CO, cellulose, glycerol and the like to yield the desired chemical product (e.g., see U.S. Pat. Nos. 8,535,916; 8,283,143; 8,268,599; and 8,110,670 for the production of fatty alcohols; see U.S. Pat. Nos. 8,110,670 and 8,313,934 for the production of fatty esters; see U.S. Pat. No. 8,372,610 for the production of odd chain fatty acid derivatives and U.S. Pat. No. 8,530,221 for the production of branched chain fatty acid derivatives; see U.S. Pat. No. 8,323,924 for the production of alkanes and alkenes; see U.S. Pat. No. 8,183,028 for the production of olefins; see U.S. Pat. No. 8,097,439 for the production of fatty aldehydes; and see U.S. Pat. No. 8,110,093 for production of low molecular weight hydrocarbons from a biocrude, all of which are incorporated herein by reference).

The present disclosure provides a further improvement by engineering environmentally friendly microorganisms that overexpress an acyl carrier protein (ACP) and express (or overexpress) a fatty acid derivative biosynthetic protein (e.g., terminal enzyme) for the production of fatty acid derivatives. The present inventors have surprisingly found that overexpressing ACP in combination with expressing or overexpressing a biosynthetic protein such as a terminal enzyme (e.g., thioesterase (TE), carboxylic acid reductase (CAR), ester synthase, acyl-ACP reductase (AAR), etc.) leads to a substantial increase in titer, yield, and/or productivity of fatty acid derivatives via the microorganisms. Such modified microorganisms can thus be characterized by a higher titer, higher yield and/or higher productivity of fatty acid derivative production when compared to their native counterparts or corresponding wild type microorganisms.

### DEFINITIONS

Unless defined otherwise, all technical and scientific terms used herein have the same meaning as commonly understood by one of ordinary skill in the art to which the disclosure pertains. Although other methods and materials similar, or equivalent, to those described herein can be used in the practice of the present disclosure, the preferred materials and methods are described herein.

As used in this specification and the appended claims, the singular forms “a,” “an” and “the” include plural referents unless the context clearly dictates otherwise. Thus, for example, reference to “a recombinant host cell” includes two or more such recombinant host cells, reference to “a fatty alcohol” includes one or more fatty alcohols, or mixtures of fatty alcohols, reference to “a nucleic acid coding sequence” includes one or more nucleic acid coding sequences, reference to “an enzyme” includes one or more enzymes, and the like.

Sequence Accession numbers throughout this description were obtained from databases provided by the NCBI (National Center for Biotechnology Information) maintained by the National Institutes of Health, U.S.A. (which are identified herein as “NCBI Accession Numbers” or alternatively as “GenBank Accession Numbers”), and from the UniProt Knowledgebase (UniProtKB) and Swiss-Prot databases provided by the Swiss Institute of Bioinformatics (which are identified herein as “UniProtKB Accession Numbers”).

Enzyme Classification (EC) Numbers are established by the Nomenclature Committee of the International Union of Biochemistry and Molecular Biology (IUBMB), a description of which is available on the IUBMB Enzyme Nomenclature website on the World Wide Web. EC numbers classify enzymes according to the enzyme-catalyzed reactions. For example, if different enzymes (e.g., from different organisms) catalyze the same reaction, then they are classified under the same EC number. In addition, through convergent evolution, different protein folds can catalyze identical reactions and therefore are assigned identical EC numbers (see Omelchenko et al. (2010) Biol. Direct 5:31). Proteins that are evolutionarily unrelated and can catalyze the same biochemical reactions are sometimes referred to as analogous enzymes (i.e., as opposed to homologous enzymes). EC numbers differ from, for example, UniProt identifiers which specify a protein by its amino acid sequence.

As used herein, the term “nucleotide” refers to a monomeric unit of a polynucleotide that consists of a heterocyclic base, a sugar, and one or more phosphate groups. The naturally occurring bases (guanine, (G), adenine, (A), cytosine, (C), thymine, (T), and uracil (U)) are typically derivatives of purine or pyrimidine, though it should be understood that naturally and non-naturally occurring base analogs are also included. The naturally occurring sugar is the pentose (five-carbon sugar) deoxyribose (which forms DNA) or ribose (which forms RNA), though it should be understood that naturally and non-naturally occurring sugar analogs are also included. Nucleic acids are typically linked via phosphate bonds to form nucleic acids or polynucleotides, though many other linkages are known in the art (e.g., phosphorothioates, boranophosphates, and the like).

As used herein, the term “polynucleotide” refers to a polymer of ribonucleotides (RNA) or deoxyribonucleotides (DNA), which can be single-stranded or double-stranded and which can contain non-natural or altered nucleotides. The terms “polynucleotide,” “nucleic acid sequence,” and “nucleotide sequence” are used interchangeably herein to refer to a polymeric form of nucleotides of any length, either RNA or DNA. These terms refer to the primary structure of the molecule, and thus include double- and single-stranded DNA, and double- and single-stranded RNA. The terms include, as equivalents, analogs of either RNA or DNA made from nucleotide analogs and modified polynucleotides such as, though not limited to methylated and/or capped polynucleotides. The polynucleotide can be in any form, including but not limited to, plasmid, viral, chromosomal, EST, cDNA, mRNA, and rRNA.

As used herein, the terms “polypeptide” and “protein” are used interchangeably to refer to a polymer of amino acid residues. The term “recombinant polypeptide” refers to a polypeptide that is produced by recombinant techniques, wherein generally DNA or RNA encoding the expressed protein is inserted into a suitable expression vector that is in turn used to transform a host cell to produce the polypeptide.

As used herein, the terms “homolog,” and “homologous” refer to a polynucleotide or a polypeptide comprising a sequence that is at least about 50% identical to the corresponding polynucleotide or polypeptide sequence. Preferably homologous polynucleotides or polypeptides have polynucleotide sequences or amino acid sequences that have at least about 80%, 81%, 82%, 83%, 84%, 85%, 86%, 87%, 88%, 89%, 90%, 91%, 92%, 93%, 94%, 95%, 96%, 97%, 98% or at least about 99% homology to the corresponding amino acid sequence or polynucleotide sequence. As used herein the terms sequence “homology” and sequence “identity” are used interchangeably. One of ordinary skill in the art is well aware of methods to determine homology between two or more sequences. Briefly, calculations of “homology” between two sequences can be performed as follows. The sequences are aligned for optimal comparison purposes (e.g., gaps can be introduced in one or both of a first and a second amino acid or nucleic acid sequence for optimal alignment and non-homologous sequences can be disregarded for comparison purposes). In one preferred embodiment, the length of a first sequence that is aligned for comparison purposes is at least about 30%, preferably at least about 40%, more preferably at least about 50%, even more preferably at least about 60%, and even more preferably at least about 70%, at least about 80%, at least about 90%, or about 100% of the length of a second sequence. The amino acid residues or nucleotides at corresponding amino acid positions or nucleotide positions of the first and second sequences are then compared. When a position in the first sequence is occupied by the same amino acid residue or nucleotide as the corresponding position in the second sequence, then the molecules are identical at that position. The percent homology between the two sequences is a function of the number of identical positions shared by the sequences, taking into account the number of gaps and the length of each gap, that need to be introduced for optimal alignment of the two sequences. The comparison of sequences and determination of percent homology between two sequences can be accomplished using a mathematical algorithm, such as BLAST (Altschul et al. (1990) J. Mol. Biol. 215(3):403-410). The percent homology between two amino acid sequences also can be determined using the Needleman and Wunsch algorithm that has been incorporated into the GAP program in the GCG software package, using either a Blossum 62 matrix or a PAM250 matrix, and a gap weight of 16, 14, 12, 10, 8, 6, or 4 and a length weight of 1, 2, 3, 4, 5, or 6 (Needleman and Wunsch (1970) J. Mol. Biol. 48:444-453). The percent homology between two nucleotide sequences also can be determined using the GAP program in the GCG software package, using a NWSgapdna.CMP matrix and a gap weight of 40, 50, 60, 70, or 80 and a length weight of 1, 2, 3, 4, 5, or 6. One of ordinary skill in the art can perform initial homology calculations and adjust the algorithm parameters accordingly. A preferred set of parameters (and the one that should be used if a practitioner is uncertain about which parameters should be applied to determine if a molecule is within a homology limitation of the claims) are a Blossum 62 scoring matrix with a gap penalty of 12, a gap extend penalty of 4, and a frameshift gap penalty of 5. Additional methods of sequence alignment are known in the biotechnology arts (see, e.g., Rosenberg (2005) BMC Bioinformatics 6:278; Altschul et al. (2005) FEBS J. 272(20):5101-5109).

As used herein, the terms “hybridizes under low stringency, medium stringency, high stringency, or very high stringency conditions” describe conditions for hybridization and washing. Guidance for performing hybridization reactions can be found in Current Protocols in Molecular Biology, John Wiley & Sons, N.Y. (1989), 6.3.1-6.3.6, which describes aqueous and non-aqueous methods. Specific hybridization conditions referred to herein are as follows: 1) low stringency hybridization conditions—6× sodium chloride/sodiumcitrate (SSC) at about 45° C., followed by two washes in 0.2×SSC, 0.1% SDS at least at 50° C. (the temperature of the washes can be increased to 55° C. for low stringency conditions); 2) medium stringency hybridization conditions—6×SSC at about 45° C., followed by one or more washes in 0.2×SSC, 0.1% SDS at 60° C.; 3) high stringency hybridization conditions—6×SSC at about 45° C., followed by one or more washes in 0.2.×SSC, 0.1% SDS at 65° C.; and 4) very high stringency hybridization conditions—0.5M sodium phosphate, 7% SDS at 65° C., followed by one or more washes at 0.2×SSC, 1% SDS at 65° C. Very high stringency conditions (4) are usually the preferred conditions unless otherwise specified.

The term “endogenous” means “originating within”. As such, an “endogenous” polypeptide refers to a polypeptide that is encoded by the native genome of the host cell. For example, an endogenous polypeptide can refer to a polypeptide that is encoded by the genome of the parental microbial cell (e.g., the parental host cell) from which the recombinant cell is engineered (or derived).

The term “exogenous” means “originating from outside”. As such, an “exogenous” polypeptide refers to a polypeptide which is not encoded by the native genome of the cell. Such an exogenous polypeptide is transferred into the cell and can be cloned from or derived from a different cell type or species; or can be cloned from or derived from the same cell type or species. For example, a variant (i.e., mutant or altered) polypeptide is an example of an exogenous polypeptide. Similarly, a non-naturally-occurring nucleic acid molecule is considered to be exogenous to a cell once introduced into the cell. The term “exogenous” may also be used with reference to a polynucleotide, polypeptide, or protein which is present in a recombinant host cell in a non-native state. For example, an “exogenous” polynucleotide, polypeptide or protein sequence may be modified relative to the wild type sequence naturally present in the corresponding wild type host cell, e.g., a modification in the level of expression or in the sequence of a polynucleotide, polypeptide or protein. Along those same lines, a nucleic acid molecule that is naturally-occurring can also be exogenous to a particular cell. For example, an entire coding sequence isolated from cell X is an exogenous nucleic acid with respect to cell Y once that coding sequence is introduced into cell Y, even if X and Y are the same cell type.

The term “overexpressed” means that a gene is caused to be transcribed at an elevated rate compared to the endogenous transcription rate for that gene. In some examples, overexpression additionally includes an elevated rate of translation of the corresponding protein compared to the endogenous translation rate for that protein. Methods of testing for overexpression are well known in the art, for example transcribed RNA levels can be assessed using rtPCR and protein levels can be assessed using SDS page gel analysis.

The term “heterologous” means “derived from a different cell, different organism, different cell type, and/or different species”. As used herein, the term “heterologous” is typically associated with a polynucleotide or a polypeptide or a protein and refers to a polynucleotide, a polypeptide or a protein that is not naturally present in a given organism, cell type, or species. For example, a polynucleotide sequence from a plant can be introduced into a microbial host cell by recombinant methods, and the plant polynucleotide is then heterologous to that recombinant microbial host cell. Similarly, a polynucleotide sequence from cyanobacteria can be introduced into a microbial host cell of the genus Escherichia by recombinant methods, and the polynucleotide from cyanobacteria is then heterologous to that recombinant microbial host cell. In some embodiments, the term “heterologous” can also be used interchangeably with the term “exogenous”. For example, an entire coding sequence isolated from cell X is a heterologous nucleic acid with respect to cell Y once that coding sequence is introduced into cell Y, even if X and Y are the same cell type.

As used herein, the term “fragment” of a polypeptide refers to a shorter portion of a full-length polypeptide or protein ranging in size from four amino acid residues to the entire amino acid sequence minus one amino acid residue. In certain embodiments of the disclosure, a fragment refers to the entire amino acid sequence of a domain of a polypeptide or protein (e.g., a substrate binding domain or a catalytic domain).

As used herein, the term “mutagenesis” refers to a process by which the genetic information of an organism is changed in a stable manner. Mutagenesis of a protein coding nucleic acid sequence produces a mutant protein. Mutagenesis also refers to changes in non-coding nucleic acid sequences that result in modified protein activity.

As used herein, the term “gene” refers to nucleic acid sequences encoding either an RNA product or a protein product, as well as operably-linked nucleic acid sequences affecting the expression of the RNA or protein (e.g., such sequences include but are not limited to promoter or enhancer sequences) or operably-linked nucleic acid sequences encoding sequences that affect the expression of the RNA or protein (e.g., such sequences include but are not limited to ribosome binding sites or translational control sequences).

Expression control sequences are known in the art and include, for example, promoters, enhancers, polyadenylation signals, transcription terminators, internal ribosome entry sites (IRES), and the like, that provide for the expression of the polynucleotide sequence in a host cell. Expression control sequences interact specifically with cellular proteins involved in transcription (Maniatis et al. (1987) Science 236:1237-1245). Exemplary expression control sequences are described in, for example, Goeddel, Gene Expression Technology: Methods in Enzymology, Vol. 185, Academic Press, San Diego, Calif. (1990). In the methods of the disclosure, an expression control sequence is operably linked to a polynucleotide sequence. By “operably linked” is meant that a polynucleotide sequence and an expression control sequence are connected in such a way as to permit gene expression when the appropriate molecules (e.g., transcriptional activator proteins) are bound to the expression control sequence. Operably linked promoters are located upstream of the selected polynucleotide sequence in terms of the direction of transcription and translation. Operably linked enhancers can be located upstream, within, or downstream of the selected polynucleotide.

As used herein, the term “vector” refers to a nucleic acid molecule capable of transporting another nucleic acid, i.e., a polynucleotide sequence, to which it has been linked. One type of useful vector is an episome (i.e., a nucleic acid capable of extra-chromosomal replication). Useful vectors are those capable of autonomous replication and/or expression of nucleic acids to which they are linked. Vectors capable of directing the expression of genes to which they are operatively linked are referred to herein as “expression vectors.” In general, expression vectors of utility in recombinant DNA techniques are often in the form of “plasmids,” which refer generally to circular double stranded DNA loops that, in their vector form, are not bound to the chromosome. Other useful expression vectors are provided in linear form. Also included are such other forms of expression vectors that serve equivalent functions and that have become known in the art subsequently hereto. In some embodiments, a recombinant vector further includes a promoter operably linked to the polynucleotide sequence. In some embodiments, the promoter is a developmentally-regulated promoter, an organelle-specific promoter, a tissue-specific promoter, an inducible promoter, a constitutive promoter, or a cell-specific promoter. The recombinant vector typically comprises at least one sequence selected from an expression control sequence operatively coupled to the polynucleotide sequence; a selection marker operatively coupled to the polynucleotide sequence; a marker sequence operatively coupled to the polynucleotide sequence; a purification moiety operatively coupled to the polynucleotide sequence; a secretion sequence operatively coupled to the polynucleotide sequence; and a targeting sequence operatively coupled to the polynucleotide sequence. In certain embodiments, the nucleotide sequence is stably incorporated into the genomic DNA of the host cell, and the expression of the nucleotide sequence is under the control of a regulated promoter region. The expression vectors as used herein include a particular polynucleotide sequence as described herein in a form suitable for expression of the polynucleotide sequence in a host cell. It will be appreciated by those skilled in the art that the design of the expression vector can depend on such factors as the choice of the host cell to be transformed, the level of expression of polypeptide desired, etc. The expression vectors described herein can be introduced into host cells to produce polypeptides, including fusion polypeptides, encoded by the polynucleotide sequences as described herein. Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes, including to increase expression of the recombinant polypeptide; to increase the solubility of the recombinant polypeptide; and to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. In certain embodiments, a polynucleotide sequence of the disclosure is operably linked to a promoter derived from bacteriophage T5.

In certain embodiments, the host cell is a yeast cell, and the expression vector is a yeast expression vector. Examples of vectors for expression in yeast S. cerevisiae include pYepSec1 (Baldari et al. (1987) EMBO J. 6:229-234); pMFa (Kurjan et al. (1982) Cell 30:933-943); pJRY88 (Schultz et al. (1987) Gene 54: 113-123); pYES2 (Invitrogen Corp., San Diego, Calif.), and picZ (Invitrogen Corp., San Diego, Calif.). In other embodiments, the host cell is an insect cell, and the expression vector is a baculovirus expression vector. Baculovirus vectors available for expression of proteins in cultured insect cells (e.g., Sf9 cells) include, for example, the pAc series (Smith et al. (1983) Mol. Cell Biol. 3:2156-2165) and the pVL series (Lucklow et al. (1989) Virology 170:31-39). In yet another embodiment, the polynucleotide sequences described herein can be expressed in mammalian cells using a mammalian expression vector. Other suitable expression systems for both prokaryotic and eukaryotic cells are well known in the art; see, e.g., Sambrook et al., “Molecular Cloning: A Laboratory Manual,” second edition, Cold Spring Harbor Laboratory, (1989).

As used herein “CoA” refers to an acyl thioester formed between the carbonyl carbon of alkyl chain and the sulfhydryl group of the 4′-phosphopantethionyl moiety of coenzyme A (CoA), which has the formula R—C(O)S-CoA, where R is any alkyl group having at least 4 carbon atoms.

The term “ACP” means acyl carrier protein. ACP is a highly conserved carrier of acyl intermediates during fatty acid biosynthesis, wherein the growing chain is bound during synthesis as a thiol ester at the distal thiol of a 4′-phosphopantetheine moiety. The protein exists in two forms, i.e., apo-ACP (inactive in fatty acid biosynthesis) and ACP or holo-ACP (active in fatty acid biosynthesis). The terms “ACP” and “holo-ACP” are used interchangeably herein and refer to the active form of the protein. An enzyme called a phosphopantetheinyltransferase is involved in the conversion of the inactive apo-ACP to the active holo-ACP. More specifically, ACP is expressed in the inactive apo-ACP form and a 4′-phosphopantetheine moiety must be post-translationally attached to a conserved serine residue on the ACP by the action of holo-acyl carrier protein synthase (ACPS), a phosphopantetheinyltransferase, in order to produce holo-ACP.

As used herein, the term “acyl-ACP” refers to an acyl thioester formed between the carbonyl carbon of an alkyl chain and the sulfhydryl group of the phosphopantetheinyl moiety of an acyl carrier protein (ACP). In some embodiments an ACP is an intermediate in the synthesis of fully saturated acyl-ACPs. In other embodiments an ACP is an intermediate in the synthesis of unsaturated acyl-ACPs. In some embodiments, the carbon chain will have about 5, 6, 7, 8, 9, 10, 11, 12, 13, 14, 15, 16, 17, 18, 19, 20, 21, 22, 23, 24, 25, or 26 carbons.

As used herein, the term “fatty acid derivative” means a “fatty acid” or a “fatty acid derivative”, which may be referred to as a “fatty acid or derivative thereof”. The term “fatty acid” means a carboxylic acid having the formula RCOOH. R represents an aliphatic group, preferably an alkyl group. R can include between about 4 and about 22 carbon atoms. Fatty acids can be saturated, monounsaturated, or polyunsaturated. A “fatty acid derivative” is a product made in part from the fatty acid biosynthetic pathway of the production host organism. “Fatty acid derivatives” includes products made in part from ACP, acyl-ACP or acyl-ACP derivatives. Exemplary fatty acid derivatives include, for example, acyl-CoA, fatty acids, fatty aldehydes, short and long chain alcohols, fatty alcohols, hydrocarbons, esters (e.g., waxes, fatty acid esters, or fatty esters), terminal olefins, internal olefins, and ketones.

A “fatty acid derivative composition” as referred to herein is produced by a recombinant host cell and typically includes a mixture of fatty acid derivatives. In some cases, the mixture includes more than one type of product (e.g., fatty acids and fatty alcohols, fatty acids and fatty acid esters or alkanes and olefins). In other cases, the fatty acid derivative compositions may include, for example, a mixture of fatty alcohols (or another fatty acid derivative) with various chain lengths and saturation or branching characteristics. In still other cases, the fatty acid derivative composition comprises a mixture of both more than one type of product and products with various chain lengths and saturation or branching characteristics.

As used herein, the term “fatty acid biosynthetic pathway” means a biosynthetic pathway that produces fatty acids and derivatives thereof. The fatty acid biosynthetic pathway may include additional enzymes to produce fatty acids derivatives having desired characteristics.

The term “fatty acid derivative biosynthetic protein” means a biosynthetic protein (e.g., enzyme) that produces fatty acids and derivatives thereof. A terminal enzyme (e.g., thioesterase (TE), carboxylic acid reductase (CAR), ester synthase, acyl-ACP reductase (AAR), decarbonylase, acyl-CoA reductase, etc.) is an example of a fatty acid biosynthetic protein. The fatty acid derivative biosynthetic protein (or combinations of such fatty acid derivative biosynthetic proteins) may produce fatty acids, fatty alcohols, fatty esters, fatty aldehydes, alkanes, alkenes, olefins, ketones and the like. In one embodiment, the fatty acid derivative biosynthetic protein has enzymatic activity. In another embodiment, the fatty acid derivative biosynthetic protein is an enzyme that can catalyze the production of a fatty acid derivative such as a fatty acid, a fatty alcohol, a fatty ester, a fatty aldehyde, an alkane, an alkene, an olefin (e.g., a terminal olefin, an internal olefin), and/or a ketone. In one particular embodiment, the fatty acid derivative biosynthetic protein has thioesterase activity or is a thioesterase in order to produce fatty acids. In another particular embodiment, the fatty acid derivative biosynthetic protein has carboxylic acid reductase (CAR) activity or is a CAR in order to produce fatty alcohols. In another particular embodiment, the fatty acid derivative biosynthetic protein has acyl-ACP reductase (AAR) activity or is an AAR in order to produce fatty alcohols and/or fatty aldehydes and/or fatty alkanes and alkenes. In another particular embodiment, the fatty acid derivative biosynthetic protein has ester synthase activity or is an ester synthase in order to produce fatty esters. In another particular embodiment, the fatty acid derivative biosynthetic protein has OleABCD activity or is an OleABCD protein in order to produce hydrocarbons such as olefins. In another particular embodiment, the fatty acid derivative biosynthetic protein has OleA activity or is an OleA protein in order to produce ketones.

As used herein, “fatty ester” means an ester having the formula RCOOR′. A fatty ester as referred to herein can be any ester made from a fatty acid, for example a fatty acid ester. In some embodiments, the R group is at least 5, at least 6, at least 7, at least 8, at least 9, at least 10, at least 11, at least 12, at least 13, at least 14, at least 15, at least 16, at least 17, at least 18, or at least 19 carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty ester composition includes one or more of a C6, C7, C8, C9, C10, C11, C12, C13, C14, C15, C16, C17, C18, C19, C20, C21, C22, C23, C24, C25, and a C26 fatty ester. In other embodiments, the fatty ester composition includes one or more of a C6, C7, C8, C9, C10, C11, C12, C13, C14, C15, C16, C17, and a C18 fatty ester. In still other embodiments, the fatty ester composition includes C12, C14, C16 and C18 fatty esters; C12, C14 and C16 fatty esters; C14, C16 and C18 fatty esters; or C12 and C14 fatty esters.

The R group of a fatty acid derivative, for example a fatty ester, can be a straight chain or a branched chain. Branched chains may have more than one point of branching and may include cyclic branches. In some embodiments, the branched fatty acid, branched fatty aldehyde, or branched fatty ester is a C6, C7, C8, C9, C10, C11, C12, C13, C14, C15, C16, C17, C18, C19, C20, C21, C22, C23, C24, C25, or a C26 branched fatty acid, branched fatty aldehyde, or branched fatty ester. In particular embodiments, the branched fatty acid, branched fatty aldehyde, or branched fatty ester is a C6, C7, C8, C9, C10, C11, C12, C13, C14, C15, C16, C17, or C18 branched fatty acid, or branched fatty ester. A fatty ester of the present disclosure may be referred to as containing an A side and a B side. As used herein, an “A side” of an ester refers to the carbon chain attached to the carboxylate oxygen of the ester. As used herein, a “B side” of an ester refers to the carbon chain comprising the parent carboxylate of the ester. When the fatty ester is derived from the fatty acid biosynthetic pathway, the A side is typically contributed by an alcohol, and the B side is contributed by a fatty acid.

As used herein, “fatty aldehyde” means an aldehyde having the formula RCHO characterized by a carbonyl group (C═O). In certain embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty aldehyde is a C6, C7, C8, C9, C10, C11, C12, C13, C14, C15, C16, C17, C18, C19, C20, C21, C22, C23, C24, C25, or a C26 fatty aldehyde. In certain embodiments, the fatty aldehyde is a C6, C7, C8, C9, C10, C11, C12, C13, C14, C15, C16, C17, or C18 fatty aldehyde.

As used herein, “fatty alcohol” means an alcohol having the formula ROH. In some embodiments, the R group is at least 5, at least 6, at least 7, at least 8, at least 9, at least 10, at least 11, at least 12, at least 13, at least 14, at least 15, at least 16, at least 17, at least 18, or at least 19, carbons in length. Alternatively, or in addition, the R group is 20 or less, 19 or less, 18 or less, 17 or less, 16 or less, 15 or less, 14 or less, 13 or less, 12 or less, 11 or less, 10 or less, 9 or less, 8 or less, 7 or less, or 6 or less carbons in length. Thus, the R group can have an R group bounded by any two of the above endpoints. For example, the R group can be 6-16 carbons in length, 10-14 carbons in length, or 12-18 carbons in length. In some embodiments, the fatty alcohol is a C6, C7, C8, C9, C10, C11, C12, C13, C14, C15, C16, C17, C18, C19, C20, C21, C22, C23, C24, C25, or a C26 fatty alcohol. In certain embodiments, the fatty alcohol is a C6, C7, C8, C9, C10, C11, C12, C13, C14″ C15, C16, C17, or C18 fatty alcohol.

As used herein, the term “alkane” means a hydrocarbon containing only single carbon-carbon bonds. The alkane may comprise from 3 to 25 carbons. In some exemplary cases, the alkane is tridecane, methyltridecane, nonadecane, methylnonadecane, heptadecane, methylheptadecane, pentadecane or methylpentadecane.

As used herein, the terms “alkene” and “olefin” are used with reference to an unsaturated chemical compound containing at least one carbon-to-carbon double bond. The alkene may comprise from 3 to 25 carbons. The olefin may be a terminal olefin or have an internal double bond.

The R group of a fatty acid derivative, for example a fatty alcohol, can be a straight chain or a branched chain and may have an even or odd number of carbons. Branched chains may have more than one point of branching and may include cyclic branches. In some embodiments, the branched fatty acid, branched fatty aldehyde, or branched fatty alcohol is a C6, C7, C8, C9, C10, C11, C12, C13, C14, C15, C16, C17, C18, C19, C20, C21, C22, C23, C24, C25, or a C26 branched fatty acid, branched fatty aldehyde, or branched fatty alcohol, respectively. In particular embodiments, the branched fatty acid, branched fatty aldehyde, or branched fatty alcohol is a C6, C7, C8, C9, C10, C11, C12, C13, C14, C15, C16, C17, or C18 branched fatty acid, branched fatty aldehyde, or branched fatty alcohol, respectively. In certain embodiments, the hydroxyl group of the branched fatty acid, branched fatty aldehyde, or branched fatty alcohol is in the primary (C1) position.

In certain embodiments, the branched fatty acid derivative is an iso-fatty acid derivative, for example an iso-fatty aldehyde, an iso-fatty alcohol, an iso-fatty ester or an antesio-fatty acid derivative, an anteiso-fatty aldehyde, an anteiso-fatty ester or an anteiso-fatty alcohol. In exemplary embodiments, the branched fatty acid derivative is selected from iso-C7:0, iso-C8:0, iso-C9:0, iso-C10:0, iso-C11:0, iso-C12:0, iso-C13:0, iso-C14:0, iso-C15:0, iso-C16:0, iso-C17:0, iso-C18:0, iso-C19:0, anteiso-C7:0, anteiso-C8:0, anteiso-C9:0, anteiso-C10:0, anteiso-C11:0, anteiso-C12:0, anteiso-C13:0, anteiso-C14:0, anteiso-C15:0, anteiso-C16:0, anteiso-C17:0, anteiso-C18:0, and an anteiso-C19:0 branched fatty aldehyde, fatty alcohol, fatty ester or fatty acid.

The R group of a branched or unbranched fatty acid derivative can be saturated or unsaturated. If unsaturated, the R group can have one or more than one point of unsaturation. In some embodiments, the unsaturated fatty acid derivative is a monounsaturated fatty acid derivative. In certain embodiments, the unsaturated fatty acid derivative is a C6:1, C7:1, C8:1, C9:1, C10:1, C11:1, C12:1, C13:1, C14:1, C15:1, C16:1, C17:1, C18:1, C19:1, C20:1, C21:1, C22:1, C23:1, C24:1, C25:1, or a C26:1 unsaturated fatty acid derivative. In certain embodiments, the unsaturated fatty acid derivative is a C10:1, C12:1, C14:1, C16:1, or C18:1 unsaturated fatty acid derivative. In other embodiments, the unsaturated fatty acid derivative is unsaturated at the omega-7 position. In certain embodiments, the unsaturated fatty acid derivative comprises a cis double bond.

As used herein, a “recombinant or engineered host cell” is a host cell (e.g., a microorganism or microbial cell) that has been modified to produce (or produce increased amounts of) one or more of fatty acid derivatives including, but not limited to, acyl-CoAs, fatty acids, short and long chain alcohols, fatty alcohols, fatty aldehydes, fatty esters (e.g., waxes, fatty acid esters, or fatty esters), hydrocarbons (e.g., terminal olefins and internal olefins), and ketones. In one preferred embodiment, the recombinant host cell encompasses increased enzymatic activity in order to produce a certain fatty acid derivative (or more of a certain fatty acid derivative). The recombinant host cell may be modified or engineered to encompass one or more such increased enzymatic activities. In other preferred embodiments, the recombinant host cell comprises one or more polynucleotides, each polynucleotide encoding a polypeptide having fatty acid derivative biosynthetic protein activity, wherein the recombinant host cell produces a fatty acid derivative composition when cultured in the presence of a carbon source under conditions effective to express the polynucleotides.

As used herein, the term “modified” or an “altered level of” a recombinant host cell refers to a difference in one or more characteristics in the activity determined relative to the parent or native host cell. Typically differences in activity are determined between a recombinant host cell, having modified activity, and the corresponding wild-type host cell (e.g., comparison of a culture of a recombinant host cell relative to the corresponding wild-type host cell). Modified activities can be the result of, for example, modified amounts of protein expressed by a recombinant host cell (e.g., as the result of increased or decreased number of copies of DNA sequences encoding the protein, increased or decreased number of mRNA transcripts encoding the protein, and/or increased or decreased amounts of protein translation of the protein from mRNA); changes in the structure of the protein (e.g., changes to the primary structure, such as, changes to the protein's coding sequence that result in changes in substrate specificity, changes in observed kinetic parameters); and changes in protein stability (e.g., increased or decreased degradation of the protein). In some embodiments, the polypeptide is a mutant or a variant of any of the polypeptides described herein. In certain instances, the coding sequences of the polypeptides described herein are codon optimized for expression in a particular host cell. For example, for expression in E. coli, one or more codons can be optimized as described in, e.g., Grosjean et al. (1982) Gene 18:199-209.

As used herein, the term “clone” typically refers to a cell or group of cells descended from and essentially genetically identical to a single common ancestor, for example, the bacteria of a cloned bacterial colony arose from a single bacterial cell.

As used herein, the term “culture” typical refers to a liquid media comprising viable cells. In one embodiment, a culture comprises cells reproducing in a predetermined culture media under controlled conditions, for example, a culture of recombinant host cells grown in liquid media comprising a selected carbon source and nitrogen.

“Culturing” or “cultivation” refers to growing a population of recombinant host cells under suitable conditions in a liquid or solid medium. In particular embodiments, culturing refers to the fermentative bioconversion of a substrate to an end-product. Culturing media are well known and individual components of such culture media are available from commercial sources, e.g., under the DIFCO and BBL labels. In one non-limiting example, the aqueous nutrient medium is a rich medium including complex sources of nitrogen, salts, and carbon, such as YP medium, encompassing about 10 g/L of peptone and 10 g/L yeast extract. Any host cell that is to be cultured can be engineered to assimilate carbon efficiently and use cellulosic materials as carbon sources according to methods described in U.S. Pat. Nos. 5,000,000; 5,028,539; 5,424,202; 5,482,846; 5,602,030; and patent application publication WO 2010127318. In addition, in some embodiments the host cell can be engineered to express an invertase so that sucrose can be used as a carbon source.

As used herein, the term “under conditions effective to express an exogenous or heterologous nucleotide sequence” means any conditions that allow a host cell (e.g., a recombinant host cell) to produce a desired fatty acid derivative. Suitable conditions include, for example, fermentation conditions.

The term “regulatory sequences” as used herein typically refers to a sequence of bases in DNA, operably-linked to DNA sequences encoding a protein that ultimately controls the expression of the protein. Examples of regulatory sequences include, but are not limited to, RNA promoter sequences, transcription factor binding sequences, transcription termination sequences, modulators of transcription (such as enhancer elements), nucleotide sequences that affect RNA stability, and translational regulatory sequences (such as, ribosome binding sites (e.g., Shine-Dalgarno sequences in prokaryotes or Kozak sequences in eukaryotes), initiation codons, termination codons).

As used herein, the phrase “the expression of said nucleotide sequence is modified relative to the wild type nucleotide sequence,” means an increase or decrease in the level of expression and/or activity of an endogenous nucleotide sequence or the expression and/or activity of an exogenous or heterologous or non-native polypeptide-encoding nucleotide sequence.

As used herein, the term “express” with respect to a polynucleotide is to cause it to function. A polynucleotide which encodes a polypeptide (or protein) will, when expressed, be transcribed and translated to produce that polypeptide (or protein). As used herein, the term “overexpress” means to express (or cause to express) a polynucleotide or polypeptide in a cell at a greater concentration than is normally expressed in a corresponding wild-type cell under the same conditions.

The terms “altered level of expression” and “modified level of expression” are used interchangeably and mean that a polynucleotide or polypeptide is present in a different concentration in an engineered host cell as compared to its concentration in a corresponding wild-type cell under the same conditions.

As used herein, the term “titer” refers to the quantity of fatty acid derivative produced per unit volume of host cell culture. In any aspect of the compositions and methods described herein, a fatty acid derivative is produced at a titer of about 25 mg/L, about 50 mg/L, about 75 mg/L, about 100 mg/L, about 125 mg/L, about 150 mg/L, about 175 mg/L, about 200 mg/L, about 225 mg/L, about 250 mg/L, about 275 mg/L, about 300 mg/L, about 325 mg/L, about 350 mg/L, about 375 mg/L, about 400 mg/L, about 425 mg/L, about 450 mg/L, about 475 mg/L, about 500 mg/L, about 525 mg/L, about 550 mg/L, about 575 mg/L, about 600 mg/L, about 625 mg/L, about 650 mg/L, about 675 mg/L, about 700 mg/L, about 725 mg/L, about 750 mg/L, about 775 mg/L, about 800 mg/L, about 825 mg/L, about 850 mg/L, about 875 mg/L, about 900 mg/L, about 925 mg/L, about 950 mg/L, about 975 mg/L, about 1000 mg/L, about 1050 mg/L, about 1075 mg/L, about 1100 mg/L, about 1125 mg/L, about 1150 mg/L, about 1175 mg/L, about 1200 mg/L, about 1225 mg/L, about 1250 mg/L, about 1275 mg/L, about 1300 mg/L, about 1325 mg/L, about 1350 mg/L, about 1375 mg/L, about 1400 mg/L, about 1425 mg/L, about 1450 mg/L, about 1475 mg/L, about 1500 mg/L, about 1525 mg/L, about 1550 mg/L, about 1575 mg/L, about 1600 mg/L, about 1625 mg/L, about 1650 mg/L, about 1675 mg/L, about 1700 mg/L, about 1725 mg/L, about 1750 mg/L, about 1775 mg/L, about 1800 mg/L, about 1825 mg/L, about 1850 mg/L, about 1875 mg/L, about 1900 mg/L, about 1925 mg/L, about 1950 mg/L, about 1975 mg/L, about 2000 mg/L (2 g/L), 3 g/L, 5 g/L, 10 g/L, 20 g/L, 30 g/L, 40 g/L, 50 g/L, 60 g/L, 70 g/L, 80 g/L, 90 g/L, 100 g/L or a range bounded by any two of the foregoing values. In other embodiments, a fatty acid derivative is produced at a titer of more than 100 g/L, more than 200 g/L, more than 300 g/L, or higher. The preferred titer of fatty acid derivative produced by a recombinant host cell according to the methods of the disclosure is from 5 g/L to 200 g/L, 10 g/L to 150 g/L, 20 g/L to 120 g/L and 30 g/L to 100 g/L. The titer may refer to a particular fatty acid derivative or a combination of fatty acid derivatives produced by a given recombinant host cell culture.

As used herein, the “yield of fatty acid derivative produced by a host cell” refers to the efficiency by which an input carbon source is converted to a product (i.e., fatty acid, fatty aldehyde, fatty alcohol, fatty ester, alkane, alkene, olefin, ketone, etc.) in a host cell. Host cells engineered to produce fatty acid derivatives according to the methods of the disclosure have a yield of at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% or a range bounded by any two of the foregoing values. In other embodiments, a fatty acid derivative or derivatives is produced at a yield of more than 30%, 40%, 50%, 60%, 70%, 80%, 90% or more. Alternatively, or in addition, the yield is about 30% or less, about 27% or less, about 25% or less, or about 22% or less. Thus, the yield can be bounded by any two of the above endpoints. For example, the yield of a fatty acid derivative or derivatives produced by the recombinant host cell according to the methods of the disclosure can be 5% to 15%, 10% to 20%, 10% to 22%, 10% to 25%, 15% to 20%, 15% to 22%, 15% to 25%, 18% to 22%, 20% to 28%, or 20% to 30%. The yield may refer to a particular fatty acid derivative or a combination of fatty acid derivatives produced by a given recombinant host cell culture.

As used herein, the term “productivity” refers to the quantity of a fatty acid derivative or derivatives produced per unit volume of host cell culture per unit time. In any aspect of the compositions and methods described herein, the productivity of a fatty acid derivative or derivatives produced by a recombinant host cell is at least 100 mg/L/hour, at least 200 mg/L/hour, at least 300 mg/L/hour, at least 400 mg/L/hour, at least 500 mg/L/hour, at least 600 mg/L/hour, at least 700 mg/L/hour, at least 800 mg/L/hour, at least 900 mg/L/hour, at least 1000 mg/L/hour, at least 1100 mg/L/hour, at least 1200 mg/L/hour, at least 1300 mg/L/hour, at least 1400 mg/L/hour, at least 1500 mg/L/hour, at least 1600 mg/L/hour, at least 1700 mg/L/hour, at least 1800 mg/L/hour, at least 1900 mg/L/hour, at least 2000 mg/L/hour, at least 2100 mg/L/hour, at least 2200 mg/L/hour, at least 2300 mg/L/hour, at least 2400 mg/L/hour, or at least 2500 mg/L/hour. For example, the productivity of a fatty acid derivative or derivatives produced by a recombinant host cell according to the methods of the may be from 500 mg/L/hour to 2500 mg/L/hour, or from 700 mg/L/hour to 2000 mg/L/hour. The productivity may refer to a particular fatty acid derivative or a combination of fatty acid derivatives produced by a given recombinant host cell culture.

As used herein, the term “total fatty species” and “total fatty acid product” may be used interchangeably herein with reference to the combined amount of fatty alcohols, fatty aldehydes, fatty esters, fatty acids, hydrocarbons, and the like, as evaluated, for example, by GC-FID. For example, when describing a fatty ester analysis, the terms “total fatty species” and “total fatty acid product” are used to refer to the combined amount of fatty esters and free fatty acids.

As used herein, the term “glucose utilization rate” means the amount of glucose used by the culture per unit time, reported as grams/liter/hour (g/L/hr).

As used herein, the term “carbon source” refers to a substrate or compound suitable to be used as a source of carbon for prokaryotic or simple eukaryotic cell growth. Carbon sources can be in various forms, including, but not limited to polymers, carbohydrates, acids, alcohols, aldehydes, ketones, amino acids, peptides, and gases (e.g., CO and CO₂). Exemplary carbon sources include, but are not limited to, monosaccharides, such as glucose, fructose, mannose, galactose, xylose, and arabinose; oligosaccharides, such as fructo-oligosaccharide and galacto-oligosaccharide; polysaccharides such as starch, cellulose, pectin, and xylan; disaccharides, such as sucrose, maltose, cellobiose, and turanose; cellulosic material and variants such as hemicelluloses, methyl cellulose and sodium carboxymethyl cellulose; saturated or unsaturated fatty acids, succinate, lactate, and acetate; alcohols, such as ethanol, methanol, and glycerol, or mixtures thereof. The carbon source can also be a product of photosynthesis, such as glucose. In certain embodiments, the carbon source is gas mixture containing CO coming from flu gas. In another embodiment, the carbon source is a gas mixture containing CO coming from the reformation of a carbon containing material, such as biomass, coal, or natural gas. In other embodiments the carbon source is syngas, methane, or natural gas. In certain preferred embodiments, the carbon source is biomass. In other preferred embodiments, the carbon source is glucose. In other preferred embodiments the carbon source is sucrose. In other embodiments the carbon source is glycerol. In other preferred embodiments the carbon source is sugar cane juice, sugar cane syrup, or corn syrup. In other preferred embodiments, the carbon source is derived from renewable feedstocks, such as CO₂, CO, glucose, sucrose, xylose, arabinose, glycerol, mannose, or mixtures thereof. In other embodiments, the carbon source is derived from renewable feedstocks including starches, cellulosic biomass, molasses, and other sources of carbohydrates including carbohydrate mixtures derived from hydrolysis of cellulosic biomass, or the waste materials derived from plant- or natural oil processing.

As used herein, the term “biomass” refers to any biological material from which a carbon source is derived. In some embodiments, a biomass is processed into a carbon source, which is suitable for bioconversion. In other embodiments, the biomass does not require further processing into a carbon source. An exemplary source of biomass is plant matter or vegetation, such as corn, sugar cane, or switchgrass. Another exemplary source of biomass is metabolic waste products, such as animal matter (e.g., cow manure). Further exemplary sources of biomass include algae and other marine plants. Biomass also includes waste products from industry, agriculture, forestry, and households, including, but not limited to, glycerol, fermentation waste, ensilage, straw, lumber, sewage, garbage, cellulosic urban waste, and food leftovers (e.g., soaps, oils and fatty acids). The term “biomass” also can refer to sources of carbon, such as carbohydrates (e.g., monosaccharides, disaccharides, or polysaccharides).

As used herein, the term “isolated,” with respect to products (such as fatty acids and derivatives thereof) refers to products that are separated from cellular components, cell culture media, or chemical or synthetic precursors. The fatty acids and derivatives thereof produced by the methods described herein can be relatively immiscible in the fermentation broth, as well as in the cytoplasm. Therefore, the fatty acids and derivatives thereof can collect in an organic phase either intracellularly or extracellularly.

As used herein, the terms “purify,” “purified,” or “purification” mean the removal or isolation of a molecule from its environment by, for example, isolation or separation. “Substantially purified” molecules are at least about 60% free (e.g., at least about 70% free, at least about 75% free, at least about 85% free, at least about 90% free, at least about 95% free, at least about 97% free, at least about 99% free) from other components with which they are associated. As used herein, these terms also refer to the removal of contaminants from a sample. For example, the removal of contaminants can result in an increase in the percentage of fatty acid derivatives in a sample. For example, when a fatty acid derivative is produced in a recombinant host cell, the fatty acid derivative can be purified by the removal of host cell proteins or other host cell materials. After purification, the percentage of fatty acid derivative in the sample is increased. The terms “purify”, “purified,” and “purification” are relative terms which do not require absolute purity. Thus, for example, when a fatty acid derivative is produced in recombinant host cells, a purified fatty acid derivative is a fatty acid derivative that is substantially separated from other cellular components (e.g., nucleic acids, polypeptides, lipids, carbohydrates, or other hydrocarbons).

Biosynthetic Pathway Engineering

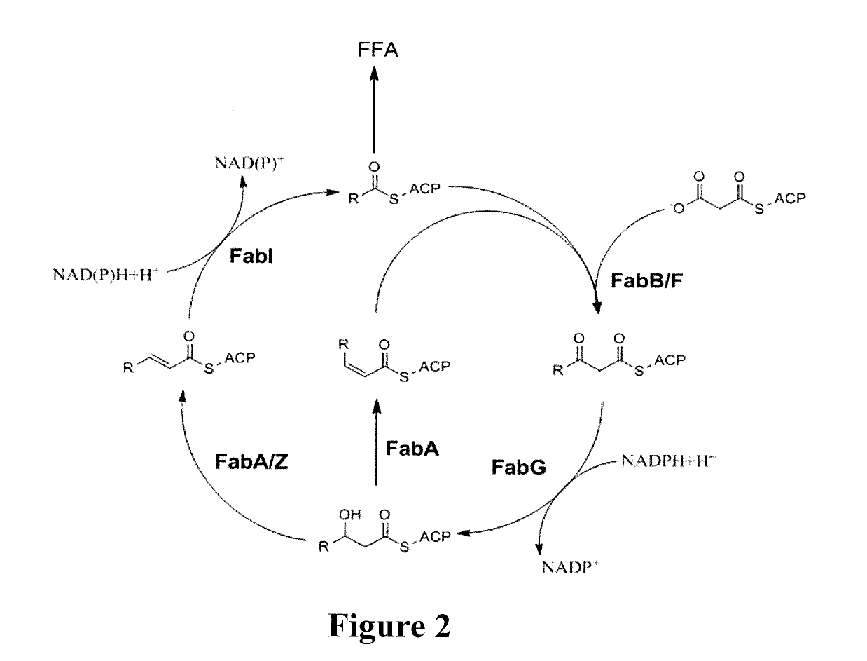

Biosynthetic pathways can be engineered or manipulated to add or remove genes that code for proteins with specific enzymatic activities in order to increase fatty acid derivative production. FIG. 2 shows an exemplary biosynthetic pathway that begins with the condensation of malonyl-ACP and acyl-ACP and ends with acyl-ACP, which provides the starting point for many engineered biochemical pathways. As shown, malonyl-ACP is produced by the transacylation of malonyl-CoA to malonyl-ACP (i.e., catalyzed by malonyl-CoA:ACP transacylase; fabD) and then β-ketoacyl-ACP synthase III (fabH) initiates condensation of malonyl-ACP with acetyl-CoA. As further shown in FIG. 2, elongation cycles begin with the condensation of malonyl-ACP and an acyl-ACP catalyzed by β-ketoacyl-ACP synthase I (fabB) and β-ketoacyl-ACP synthase II (fabF) to produce a β-keto-acyl-ACP. Then the β-keto-acyl-ACP is reduced by a NADPH-dependent β-ketoacyl-ACP reductase (fabG) to produce a β-hydroxy-acyl-ACP, which is dehydrated to a trans-2-enoyl-acyl-ACP by β-hydroxyacyl-ACP dehydratase (fabA or fabZ). FabA can also isomerize trans-2-enoyl-acyl-ACP to cis-3-enoyl-acyl-ACP, which can bypass fabI and can be used by fabB (typically for up to an aliphatic chain length of C16) to produce β-keto-acyl-ACP. The final step in each cycle is catalyzed by a NADH or NADHPH-dependent enoyl-ACP reductase (fabI) that converts trans-2-enoyl-acyl-ACP to acyl-ACP.

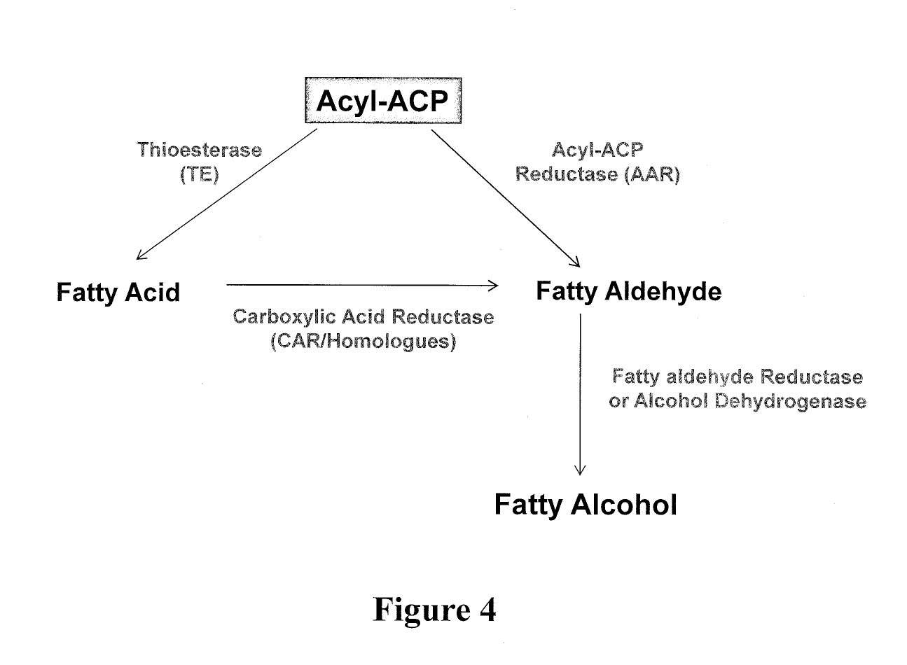

In the methods described herein, termination of fatty acid biosynthesis occurs by thioesterase removal of the acyl group from acyl-ACP to release free fatty acids (FFA). Herein, thioesterases hydrolyze thioester bonds, which occur between acyl chains and ACP through sulfhydryl bonds. Thus, fatty acid derivative production can be increased by up-regulating or overexpressing a thioesterase leading to a higher production of fatty acids. If a thioesterase is overexpressed in combination with other fatty acid derivative biosynthetic enzymes such as carboxylic acid reductase (CAR) then the pathway will lead to an increased amount of fatty aldehydes. As shown in FIG. 4, an exemplary biosynthetic pathway for the production of a fatty alcohol begins with the production of a fatty aldehyde which is catalyzed by the enzymatic activity of an acyl-ACP reductase (AAR); or a thioesterase in combination with a carboxylic acid reductase (CAR). The fatty aldehyde can then be converted to a fatty alcohol by a fatty aldehyde reductase activity (also referred to as alcohol dehydrogenase activity).

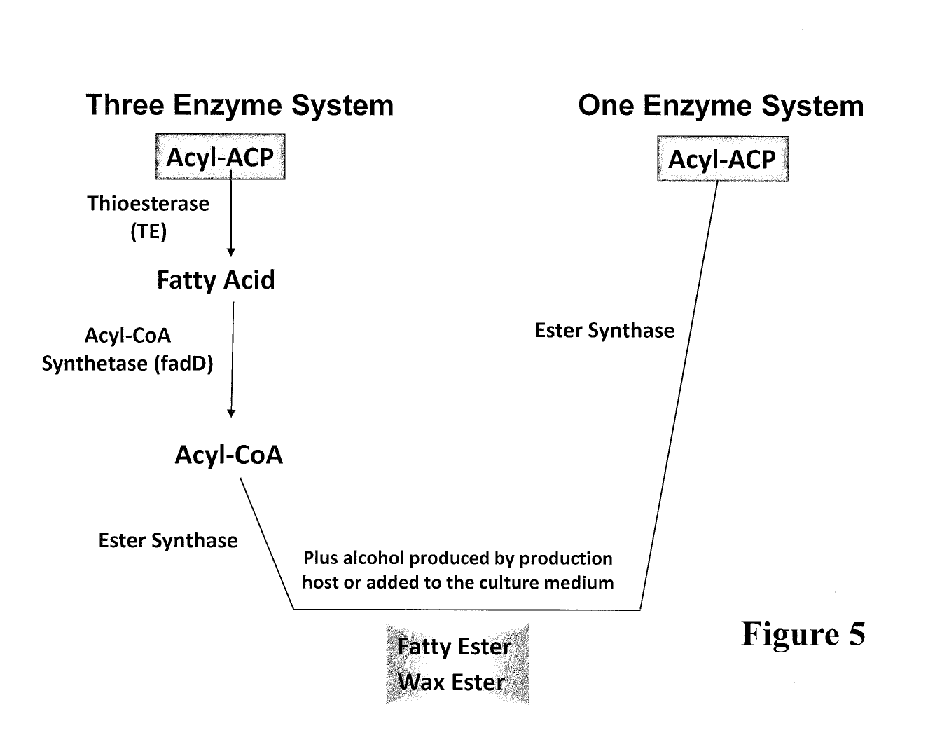

Another example of an engineered biosynthetic pathway that begins with Acyl-ACP is shown in FIG. 5, wherein fatty esters are produced via two alternative routes. As shown, one exemplary biosynthetic pathway employs one enzyme system (i.e., ester synthase) to produce fatty esters. Another exemplary biosynthetic pathway uses a three enzyme system (i.e., thioesterase (TE), acyl-CoA synthetase (FadD), and ester synthase (ES)) in order to produce fatty esters.

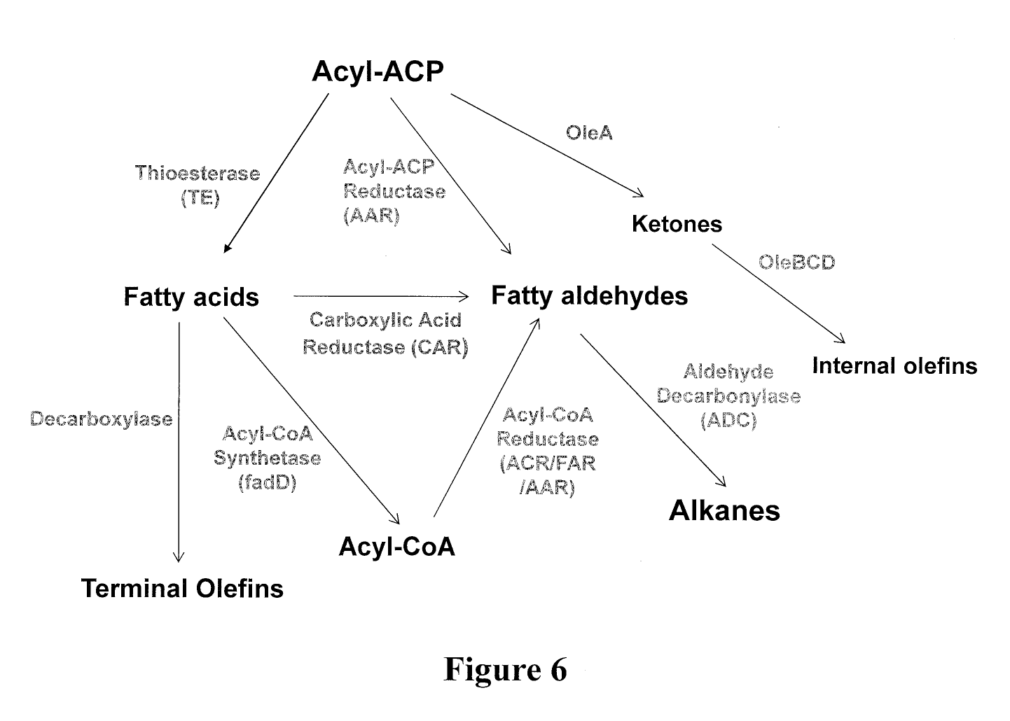

Yet, another exemplary biosynthetic pathway that beings with acyl-ACP is the production of hydrocarbons. As shown in FIG. 6, the production of internal olefins is catalyzed by the enzymatic activity of OleABCD. The production of alkanes is catalyzed by the enzymatic conversion of acyl-ACP to fatty aldehydes by AAR, and then by the enzymatic conversion of fatty aldehydes to alkanes by way of aldehyde decarbonylase (ADC). The production of terminal olefins is catalyzed by the enzymatic conversion of fatty acids to terminal olefins by a decarboxylase. In addition, the production of ketones is catalyzed by the enzymatic activity of OleA, which converts acyl-ACP to aliphatic ketones.

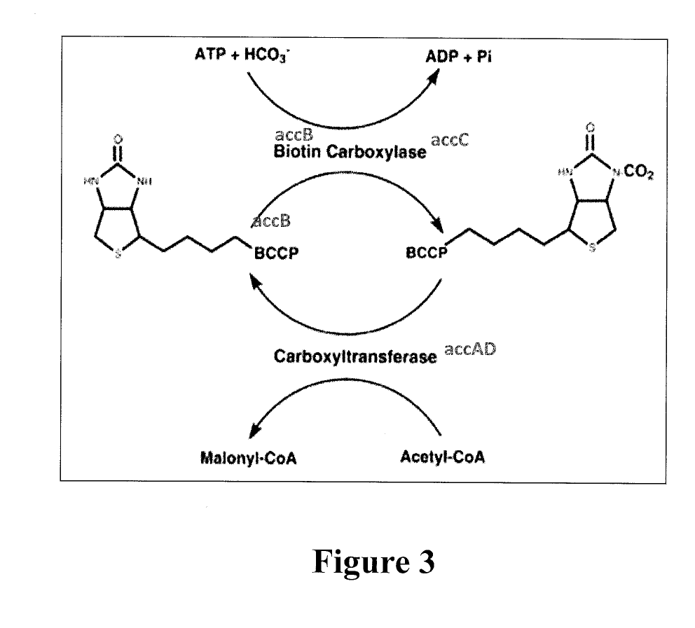

Fatty acid derivative production such as the production of fatty acid, fatty alcohols, fatty esters, fatty aldehydes, and the like, can be further increased by up-regulating or overexpressing acetyl-CoA carboxylase. This occurs because ACC produces malonyl-CoA which is then converted to malonyl-ACP which is the substrate by which all fatty acyl compounds are made through cyclic elongation of acetoacetyl-ACP initiation molecules. FIG. 3 illustrates the structure and function of the acetyl-CoA carboxylase enzyme complex (encoded by the accABCD gene). Biotin carboxylase is encoded by the accC gene, whereas biotin carboxyl carrier protein (BCCP) is encoded by the accB gene. The two subunits involved in carboxyl transferase activity are encoded by the accA and accD genes. The covalently bound biotin of BCCP carries the carboxylate moiety. The birA gene product birA biotinylates holo-accB (see FIG. 3). BirA stands for bifunctional biotin-[acetyl-CoA-carboxylase] ligase and transcriptional repressor. As such, birA is a bifunctional protein that exhibits biotin ligase activity and also acts as the DNA binding transcriptional repressor of the biotin operon.

Effect of Increasing ACP on Fatty Acid Derivative Production

The present disclosure provides recombinant microorganisms that overexpress an acyl carrier protein (ACP) and a fatty acid derivative biosynthetic protein for the production of fatty acid derivatives. These modified microorganisms can be characterized by a higher titer, higher yield and/or higher productivity of fatty acid derivative production when compared to their native counterparts or corresponding wild type microorganisms.

In order to illustrate the disclosure, microorganisms (e.g., microbial cells) have been modified to overexpress an ACP and a fatty acid derivative biosynthetic protein in order to increase the production of fatty acid derivatives (see Examples, infra). The supply of acyl-ACPs from acetyl-CoA via the acetyl-CoA carboxylase (ACC) complex and the fatty acid biosynthetic (Fab) pathway can impact the rate of fatty acid and fatty acid derivative production in a native cell. One approach to increasing the flux through fatty acid biosynthesis is to manipulate various enzymes in the Fab pathway and/or increase the amount of a rate-limiting starting material such as ACP. Although ACP proteins are conserved to some extent in all organisms, their primary sequence can differ. It has been suggested that when terminal pathway enzymes from sources other than Escherichia coli (E. coli) are expressed in E. coli in order to convert fatty acyl-ACPs to products, limitations may exist such as in the recognition, affinity and/or turnover of the recombinant pathway enzyme towards the fatty acyl-ACPs (see Suh et al. (1999) The Plant Journal 17(6):679-688; Salas et al. (2002) Archives of Biochemistry and Biophysics 403:25-34).

However, ACPs are known to play an important role in the elongation of fatty acids. For example, E. coli ACP (ecACP), encoded by the acpP gene, carries fatty acid chains via a thioester linkage to a phosphopantetheine prosthetic group as the chains are elongated. While not wishing to be bound by theory, it is proposed herein that overexpression of ACP genes may be effective in increasing the amount of acyl-ACPs, which may have a positive impact on the level of efficiency of fatty acid biosynthesis and elongation. For example, the product output in the cells depends to some degree on the availability of acyl-ACP, thus, increasing ACP expression is believed to increase the number of acyl-ACP molecules in a cell, leading to more fatty acid derivative product, since a higher number of acyl-chains would be elongated by the fatty acid biosynthetic machinery. Increasing the expression of ACPs may also de-regulate fatty acid biosynthesis at different nodes, such as, for example, ACC, fabH, and/or fabI. The enzymes ACC, fabH and/or fabI are believed to be inhibited by long chain acyl-ACP (see Davis et al. (2001) Journal of Bacteriology 183(4):1499-1503; Heath et al. (1996) The Journal of Biological Chemistry 271(4):1833-1836; and Heath et al. (1996) The Journal of Biological Chemistry 271(18):10996-11000). Thus, the accumulation of long chain acyl-ACP would slow down the production of fatty acid derivatives. Increasing the availability of ACP could de-inhibit ACC, fabH and/or fabI which, in turn, should increase fatty acid derivative output.

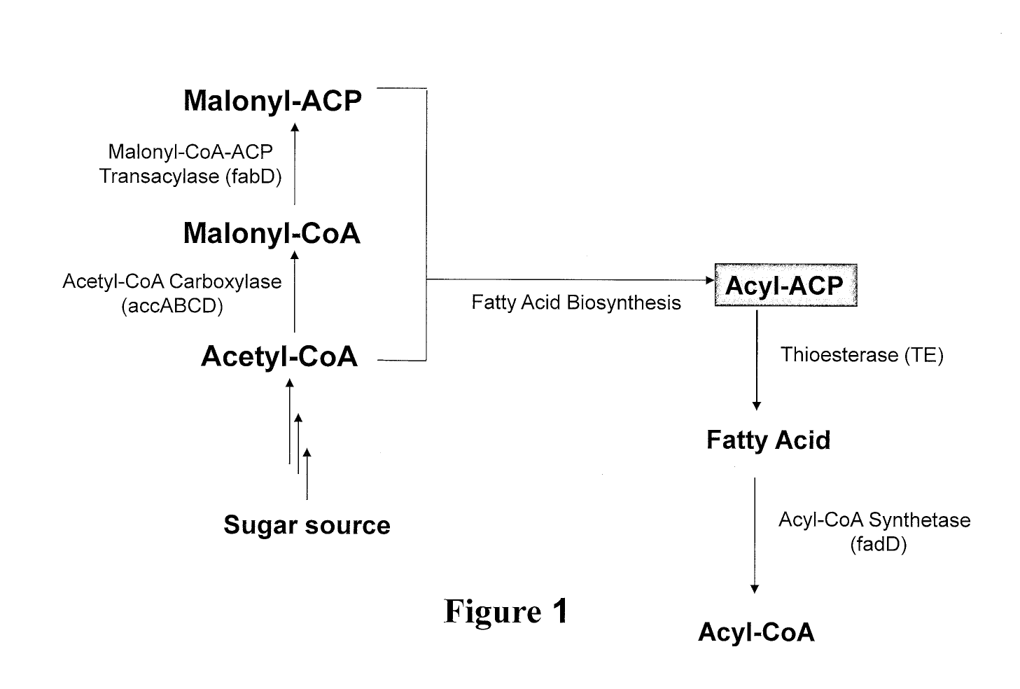

The compounds acetyl-CoA and malonyl-CoA are important precursors for fatty acid biosynthesis. When the availability of these precursors in the cell is reduced, it can result in decreased synthesis of fatty acid derivatives. One approach to increasing the flux through fatty acid biosynthesis is to manipulate various enzymes in the pathway (see FIGS. 1-3). The supply of acyl-ACPs from acetyl-CoA via the acetyl-CoA carboxylase (ACC) complex and the fatty acid biosynthetic (Fab) pathway may impact the rate of fatty acid derivative production (see FIG. 2). The effect of overexpression of ACP on production of fatty acid derivatives was tested in Examples 1-4 (infra). Surprisingly, the cells showed a significant increase in final product output, i.e., fatty acid derivative production. This was unexpected because overexpression of ACP (which is one of the most abundant proteins inside the cell) has been shown to inhibit cell growth in E. coli, i.e., within 3 to 4 hours of overexpressing ACP by about 20 fold the growth rate of E. coli cells ceased completely (see Keating et al. (1995) The Journal of Biological Chemistry 270(38):22229-22235). It has previously been determined that when ACP is overproduced from a multi-copy plasmid, the cellular capacity for post-translational modification of ACP becomes rate-limiting and apo-ACP (the inactive form) accumulates in the cell, thereby most likely leading to toxicity since wild type cells have no detectable pools of apo-ACP (see Keating, supra). Thus, it was expected that increasing ACP expression would result in the previously observed cellular feedback inhibition and limited growth. Instead, the cells overexpressing ACP showed a significant increase in fatty acid derivative production (see Examples 1-4 (infra)).

A recombinant ACP-expressing host cell can exhibit an increase in titer of a fatty acid derivative composition or a specific fatty acid derivative wherein the increase is at least 3%, at least 4%, at least 5%, at least 6%, at least 7%, at least 8%, at least 9%, at least 10%, at least 11%, at least 12%, at least 13%, at least 14%, at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, or at least 30% greater than the titer of the fatty acid derivative composition or specific fatty acid derivative produced by a corresponding host cell that does not express ACP when cultured under the same conditions. The production of increased fatty acid derivatives by ACP-expressing host cells has been confirmed (see Examples 1-4, infra), wherein increased amounts of fatty acid derivatives, including fatty acids, fatty esters, fatty alcohols, and alkanes were made.

ACP Proteins

In one aspect the disclosure relates to improved production of fatty acid derivatives such as, for example, fatty alcohols and/or fatty esters by engineering a host cell to express a native (endogenous) or non-native (exogenous or heterologous) ACP protein. The ACP polypeptide or the polynucleotide sequence that encodes the ACP polypeptide may be non-native or exogenous or heterologous, i.e., it may differ from the wild type sequence naturally present in the corresponding wild type host cell. Examples include a modification in the level of expression or in the sequence of a nucleotide, polypeptide or protein. The disclosure includes ACP polypeptides and homologs thereof.

In one embodiment, an ACP polypeptide for use in practicing the disclosure has at least 70% sequence identity to SEQ ID NO: 2, SEQ ID NO: 4, SEQ ID NO: 6, SEQ ID NO: 8 or SEQ ID NO: 10. In some embodiments the ACP is derived from a Marinobacter species or E. coli. In other embodiments, an ACP polypeptide for use in practicing the disclosure has at least 75% (e.g., at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or at least 99%) sequence identity to the wild-type ACP polypeptide sequence of SEQ ID NO: 2, SEQ ID NO: 4, SEQ ID NO: 6, SEQ ID NO: 8 or SEQ ID NO: 10, and may also include one or more substitutions which results in useful characteristics and/or properties as described herein. In one aspect of the disclosure, an ACP polypeptide for use in practicing the disclosure has 100% sequence identity to SEQ ID NO: 2, SEQ ID NO: 4, SEQ ID NO: 6, SEQ ID NO: 8 or SEQ ID NO: 10. In other embodiments, the improved or variant ACP polypeptide sequence is derived from a species other than M. hydrocarbonoclasticus or E. coli. In a related aspect, an ACP polypeptide for use in practicing the disclosure is encoded by a nucleotide sequence having 100% sequence identity to SEQ ID NO: 1, SEQ ID NO: 3, SEQ ID NO: 5, SEQ ID NO: 7, or SEQ ID NO: 9. In a related aspect, the disclosure relates to ACP polypeptides that comprise an amino acid sequence encoded by a nucleic acid sequence that has at least 75% (e.g., at least 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98% or and at least 99%) sequence identity to SEQ ID NO: 1, SEQ ID NO: 3, SEQ ID NO: 5, SEQ ID NO: 7, or SEQ ID NO: 9. In some embodiments the nucleic acid sequence encodes an ACP variant with one or more substitutions which results in improved characteristics and/or properties as described herein. In other embodiments, the improved or variant ACP nucleic acid sequence is derived from a species other than M. hydrocarbonoclasticus or E. coli. In another aspect, the disclosure relates to ACP polypeptides that comprise an amino acid sequence encoded by a nucleic acid that hybridizes under stringent conditions over substantially the entire length of a nucleic acid corresponding to SEQ ID NO: 1, SEQ ID NO: 3, SEQ ID NO: 5, SEQ ID NO: 7, or SEQ ID NO: 9. In some embodiments the nucleic acid sequence encodes an improved or variant ACP nucleic acid sequence derived from a species other than Marinobacter hydrocarbonoclasticus or E. coli.

ACP Mutants and Variants

In some embodiments, the ACP polypeptide is a mutant or a variant of any of the polypeptides described herein. The terms “mutant” and “variant” as used herein refer to a polypeptide having an amino acid sequence that differs from a wild-type polypeptide by at least one amino acid. For example, the mutant can comprise one or more of the following conservative amino acid substitutions such as replacement of an aliphatic amino acid (e.g., alanine, valine, leucine, and isoleucine), with another aliphatic amino acid; replacement of a serine with a threonine; replacement of a threonine with a serine; replacement of an acidic residue, such as aspartic acid and glutamic acid, with another acidic residue; replacement of a residue bearing an amide group, such as asparagine and glutamine, with another residue bearing an amide group; exchange of a basic residue, such as lysine and arginine, with another basic residue; and replacement of an aromatic residue, such as phenylalanine and tyrosine, with another aromatic residue. In some embodiments, the mutant polypeptide has about 1, 2, 3, 4, 5, 6, 7, 8, 9, 10, 15, 20, 30, 40, 50, 60, 70, 80, 90, 100, or more amino acid substitutions, additions, insertions, or deletions. Preferred fragments or mutants of a polypeptide retain some or all of the biological function (e.g., enzymatic activity) of the corresponding wild-type polypeptide. In some embodiments, the fragment or mutant retains at least 75%, at least 80%, at least 90%, at least 95%, or at least 98% or more of the biological function of the corresponding wild-type polypeptide. In other embodiments, the fragment or mutant retains about 100% of the biological function of the corresponding wild-type polypeptide. Guidance in determining which amino acid residues may be substituted, inserted, or deleted without affecting biological activity may be found using computer programs well known in the art, for example, the LASERGENE software (DNASTAR, Inc., Madison, Wis.). In still other embodiments, a fragment or mutant exhibits increased biological function as compared to a corresponding wild-type polypeptide. For example, a fragment or mutant may display at least a 10%, at least a 25%, at least a 50%, at least a 75%, or at least a 90% improvement in enzymatic activity as compared to the corresponding wild-type polypeptide. In other embodiments, the fragment or mutant displays at least a 100% or at least a 200%, or at least a 500% improvement in enzymatic activity as compared to the corresponding wild-type polypeptide.

It is understood that the polypeptides described herein may have additional conservative or non-essential amino acid substitutions, which do not have a substantial effect on the polypeptide function. Whether or not a particular substitution will be tolerated (i.e., will not adversely affect desired biological function, such as ACP activity) can be determined as described in the art (see Bowie et al. (1990) Science 247:1306-1310). A “conservative amino acid substitution” is one in which the amino acid residue is replaced with an amino acid residue having a similar side chain. Families of amino acid residues having similar side chains have been defined in the art. These families include amino acids with basic side chains (e.g., lysine, arginine, histidine), acidic side chains (e.g., aspartic acid, glutamic acid), uncharged polar side chains (e.g., glycine, asparagine, glutamine, serine, threonine, tyrosine, cysteine), nonpolar side chains (e.g., alanine, valine, leucine, isoleucine, proline, phenylalanine, methionine, tryptophan), beta-branched side chains (e.g., threonine, valine, isoleucine), and aromatic side chains (e.g., tyrosine, phenylalanine, tryptophan, histidine).

Variants can be naturally occurring or created in vitro. In particular, such variants can be created using genetic engineering techniques, such as site directed mutagenesis, random chemical mutagenesis, Exonuclease III deletion procedures, or standard cloning techniques. Alternatively, such variants, fragments, analogs, or derivatives can be created using chemical synthesis or modification procedures. Methods of making variants are well known in the art. These include procedures in which nucleic acid sequences obtained from natural isolates are modified to generate nucleic acids that encode polypeptides having characteristics that enhance their value in industrial or laboratory applications. In such procedures, a large number of variant sequences having one or more nucleotide differences with respect to the sequence obtained from the natural isolate are generated and characterized. Typically, these nucleotide differences result in amino acid changes with respect to the polypeptides encoded by the nucleic acids from the natural isolates. For example, variants can be prepared by using random and site-directed mutagenesis. Random and site-directed mutagenesis is known in the art (see Arnold Curr. Opin. Biotech. (1993) 4:450-455). Random mutagenesis can be achieved using error prone PCR (see Leung et al. (1989) Technique 1:11-15); and Caldwell et al. (1992) PCR Methods Applic. 2:28-33). In error prone PCR, the actual PCR is performed under conditions where the copying fidelity of the DNA polymerase is low, such that a high rate of point mutations is obtained along the entire length of the PCR product. Briefly, in such procedures, nucleic acids to be mutagenized (e.g., a polynucleotide sequence encoding an ACP) are mixed with PCR primers, reaction buffer, MgCl₂, MnCl₂, Taq polymerase, and an appropriate concentration of dNTPs for achieving a high rate of point mutation along the entire length of the PCR product. For example, the reaction can be performed using 20 fmoles of nucleic acid to be mutagenized, 30 pmole of each PCR primer, a reaction buffer comprising 50 mMKCl, 10 mM Tris HCl (pH 8.3), 0.01% gelatin, 7 mM MgCl₂, 0.5 mM MnCl₂, 5 units of Taq polymerase, 0.2 mM dGTP, 0.2 mM dATP, 1 mM dCTP, and 1 mM dTTP. PCR can be performed for 30 cycles of 94° C. for 1 min, 45° C. for 1 min, and 72° C. for 1 min. However, it will be appreciated that these parameters can be varied as appropriate. The mutagenized nucleic acids are then cloned into an appropriate vector, and the activities of the polypeptides encoded by the mutagenized nucleic acids are evaluated. Site-directed mutagenesis can also be achieved using oligonucleotide-directed mutagenesis to generate site-specific mutations in any cloned DNA of interest. Oligonucleotide mutagenesis is described in the art (see Reidhaar-Olson et al. (1988) Science 241:53-57). Briefly, in such procedures a plurality of double stranded oligonucleotides bearing one or more mutations to be introduced into the cloned DNA are synthesized and inserted into the cloned DNA to be mutagenized (e.g., a polynucleotide sequence encoding a CAR polypeptide). Clones containing the mutagenized DNA are recovered, and the activities of the polypeptides they encode are assessed.

Another method for generating variants is assembly PCR. Assembly PCR involves the assembly of a PCR product from a mixture of small DNA fragments. A large number of different PCR reactions occur in parallel in the same vial, with the products of one reaction priming the products of another reaction. Assembly PCR is described in, for example, U.S. Pat. No. 5,965,408. Still another method of generating variants is sexual PCR mutagenesis (see Stemmer (1994) Proc. Natl. Acad. Sci., U.S.A. 91:10747-10751). In sexual PCR mutagenesis, forced homologous recombination occurs between DNA molecules of different, but highly related, DNA sequences in vitro as a result of random fragmentation of the DNA molecule based on sequence homology. This is followed by fixation of the crossover by primer extension in a PCR reaction.

Variants can also be created by in vivo mutagenesis. In some embodiments, random mutations in a nucleic acid sequence are generated by propagating the sequence in a bacterial strain, such as an E. coli strain, which carries mutations in one or more of the DNA repair pathways. Such “mutator” strains have a higher random mutation rate than that of a wild-type strain. Propagating a DNA sequence (e.g., a polynucleotide sequence encoding a CAR polypeptide) in one of these strains will eventually generate random mutations within the DNA. Mutator strains suitable for use for in vivo mutagenesis are described in, for example, International Patent Application Publication No. WO 1991/016427. Variants can also be generated using cassette mutagenesis. In cassette mutagenesis, a small region of a double-stranded DNA molecule is replaced with a synthetic oligonucleotide cassette that differs from the native sequence. The oligonucleotide often contains a completely and/or partially randomized native sequence. Recursive ensemble mutagenesis can also be used to generate variants. Recursive ensemble mutagenesis is an algorithm for protein engineering (i.e., protein mutagenesis) developed to produce diverse populations of phenotypically related mutants whose members differ in amino acid sequence. This method uses a feedback mechanism to control successive rounds of combinatorial cassette mutagenesis. Recursive ensemble mutagenesis is known in the art (see Arkin et al. (1992) Proc. Natl. Acad. Sci., U.S.A. 89:7811-7815). In some embodiments, variants are created using exponential ensemble mutagenesis (see Delegrave et al. (1993) Biotech. Res. 11:1548-1552). Exponential ensemble mutagenesis is a process for generating combinatorial libraries with a high percentage of unique and functional mutants, wherein small groups of residues are randomized in parallel to identify, at each altered position, amino acids which lead to functional proteins. In some embodiments, variants are created using shuffling procedures wherein portions of a plurality of nucleic acids that encode distinct polypeptides are fused together to create chimeric nucleic acid sequences that encode chimeric polypeptides as described in, for example, U.S. Pat. Nos. 5,965,408 and 5,939,250.

Production of Fatty Acid Derivatives

This disclosure provides numerous examples of polypeptides (i.e., enzymes) having activities suitable for use in the fatty acid biosynthetic pathways as described herein. Such polypeptides are collectively referred to herein as fatty acid biosynthetic polypeptides or proteins or fatty acid biosynthetic enzymes. Non-limiting examples of fatty acid pathway polypeptides suitable for use in recombinant host cells of the disclosure are provided herein. In some embodiments, the disclosure includes a recombinant host cell comprising a polynucleotide sequence (also referred to herein as a fatty acid biosynthetic polynucleotide sequence) which encodes a fatty acid biosynthetic polypeptide. The polynucleotide sequence, which comprises an open reading frame encoding a fatty acid biosynthetic polypeptide and operably-linked regulatory sequences, can be integrated into a chromosome of the recombinant host cells, incorporated in one or more plasmid expression systems resident in the recombinant host cell, or both. Examples of biosynthetic polypeptides or proteins that can be expressed in combination with ACP are carboxylic acid reductase (CAR), thioesterase (TE), acyl-ACP reductase (AAR), acyl-CoA reductase (ACR), ester synthase (ES), decarbonylase, acetyl-CoA carboxylase (ACC), fatty alcohol forming acyl-CoA reductase (FAR), and others (see also Table 1, infra). In Examples 1-4 (infra), both plasmid expression systems and integration into the host genome are used to illustrate different embodiments of the present disclosure.

In some embodiments, a fatty acid biosynthetic polynucleotide sequence encodes a polypeptide which is endogenous to the parental host cell of the recombinant cell being engineered. In other embodiments, a fatty acid biosynthetic polynucleotide sequence encodes a polypeptide which is exogenous to the parental host cell of the recombinant cell being engineered. In still other embodiments, a fatty acid biosynthetic polynucleotide sequence encodes a polypeptide which is heterologous to the parental host cell of the recombinant cell being engineered. In still other embodiments, a fatty acid biosynthetic polynucleotide sequence encodes an exogenous or heterologous polypeptide which is expressed in the recombinant cell when compared to the corresponding parent host cell. In yet other embodiments, a fatty acid biosynthetic polynucleotide sequence encodes an endogenous polypeptide which is overexpressed in the recombinant cell when compared to the corresponding parent host cell. In certain embodiments, the enzyme encoded by the overexpressed gene is directly involved in fatty acid biosynthesis. In some embodiments, at least one polypeptide encoded by a fatty acid biosynthetic polynucleotide is an exogenous or heterologous polypeptide. In other embodiments, at least one polypeptide encoded by a fatty acid biosynthetic polynucleotide is an overexpressed polypeptide. Table 1 provides a listing of exemplary proteins which can be expressed or overexpressed in recombinant host cells to facilitate production of particular fatty acid derivatives.

TABLE 1
Gene Designations

| | | | | | |
|---|---|---|---|---|---|
| Gene | Source | | Accession | EC | |
| Designation | Organism | Enzyme Name | No. | Number | Exemplary Use |

Fatty Acid Production Increase/Product Production Increase

| | | | | | |
|---|---|---|---|---|---|
| accA | E. coli, | acetyl-CoA | AAC73296, | 6.4.1.2 | increase Malonyl- |
| | Lactococci | carboxylase, subunit A | NP_414727 | | CoA production |
| | | (carboxyltransferase | | | |
| | | alpha) | | | |
| accB | E. coli, | acetyl-CoA | NP_417721 | 6.4.1.2 | increase Malonyl- |
| | Lactococci | carboxylase, subunit B | | | CoA production |
| | | (BCCP: biotin | | | |
| | | carboxyl carrier | | | |
| | | protein) | | | |
| accC | E. coli, | acetyl-CoA | NP_417722 | 6.4.1.2, | increase Malonyl- |
| | Lactococci | carboxylase, subunit C | | 6.3.4.14 | CoA production |
| | | (biotin carboxylase) | | | |
| accD | E. coli, | acetyl-CoA | NP_416819 | 6.4.1.2 | increase Malonyl- |
| | Lactococci | carboxylase, subunit D | | | CoA production |
| | | (carboxyltransferase | | | |
| | | beta) | | | |
| fadD | E. coli W3110 | acyl-CoA synthase | AP_002424 | 2.3.1.86, | increase Fatty acid |
| | | | | 6.2.1.3 | production |
| fabA | E. coli K12 | β- | NP_415474 | 4.2.1.60 | increase fatty acyl- |
| | | hydroxydecanoylthioesterdehydratase/ | | | ACP/CoA |
| | | isomerase | | | production |
| fabB | E. coli | 3-oxoacyl-[acyl- | BAA16180 | 2.3.1.41 | increase fatty acyl- |
| | | carrier-protein] | | | ACP/CoA |
| | | synthase I | | | production |
| fabD | E. coli K12 | [acyl-carrier-protein] | AAC74176 | 2.3.1.39 | increase fatty acyl- |
| | | S-malonyltransferase | | | ACP/CoA |
| | | | | | production |
| fabF | E. coli K12 | 3-oxoacyl-[acyl- | AAC74179 | 2.3.1.179 | increase fatty acyl- |
| | | carrier-protein] | | | ACP/CoA |
| | | synthase II | | | production |
| fabG | E. coli K12 | 3-oxoacyl-[acyl-carrier | AAC74177 | 1.1.1.100 | increase fatty acyl- |
| | | protein] reductase | | | ACP/CoA |
| | | | | | production |
| fabH | E. coli K12 | 3-oxoacyl-[acyl- | AAC74175 | 2.3.1.180 | increase fatty acyl- |
| | | carrier-protein] | | | ACP/CoA |
| | | synthase III | | | production |
| fabI | E. coli K12 | enoyl-[acyl-carrier- | NP_415804 | 1.3.1.9 | increase fatty acyl- |
| | | protein] reductase | | | ACP/CoA |
| | | | | | production |
| fabR | E. coli K12 | transcriptional | NP_418398 | none | modulate |
| | | Repressor | | | unsaturated fatty |
| | | | | | acid production |
| fabV | Vibrio cholerae | enoyl-[acyl-carrier- | YP_001217283 | 1.3.1.9 | increase fatty acyl- |
| | | protein] reductase | | | ACP/CoA |
| | | | | | production |
| fabZ | E. coli K12 | (3R)-hydroxymyristol | NP_414722 | 4.2.1.— | increase fatty acyl- |
| | | acyl carrier protein | | | ACP/CoA |
| | | dehydratase | | | production |
| fadE | E. coli K13 | acyl-CoA | AAC73325 | 1.3.99.3, | reduce fatty acid |
| | | dehydrogenase | | 1.3.99.— | degradation |
| fadR | E. coli | transcriptional | NP_415705 | none | Block or reverse |
| | | regulatory protein | | | fatty acid |
| | | | | | degradation |

Chain Length Control

| | | | | | |
|---|---|---|---|---|---|
| tesA (with or | E. coli | thioesterase - leader | P0ADA1 | 3.1.2.—, | C18 Chain Length |
| without | | sequence is amino | | 3.1.1.5 | |
| leader | | acids 1-26 | | | |
| sequence) | | | | | |
| tesA | E. coli | thioesterase | AAC73596, | 3.1.2.—, | C18:1 Chain Length |
| (without | | | NP_415027 | 3.1.1.5 | |
| leader | | | | | |
| sequence) | | | | | |
| tesA (mutant | E. coli | thioesterase | L109P | 3.1.2.—, | <C18 Chain Length |
| of E. coli | | | | 3.1.1.5 | |
| thioesterase I | | | | | |
| complexed | | | | | |
| with | | | | | |
| octanoic | | | | | |
| acid) | | | | | |
| fatB1 | Umbellulariaca | thioesterase | Q41635 | 3.1.2.14 | C12:0 Chain Length |
| | lifornica | | | | |
| fatB2 | Cuphea hookeriana | thioesterase | AAC49269 | 3.1.2.14 | C8:0-C10:0 Chain |
| | | | | | Length |
| fatB3 | Cuphea hookeriana | thioesterase | AAC72881 | 3.1.2.14 | C14:0-C16:0 Chain |
| | | | | | Length |
| fatB | Cinnamomum camphora | thioesterase | Q39473 | 3.1.2.14 | C14:0 Chain Length |
| fatB | Arabidopsis | thioesterase | CAA85388 | 3.1.2.14 | C16:1 Chain Length |
| | thaliana | | | | |
| fatA1 | Helianthus | thioesterase | AAL79361 | 3.1.2.14 | C18:1 Chain Length |
| | annuus | | | | |
| atfata | Arabidopsis | thioesterase | NP_189147, | 3.1.2.14 | C18:1 Chain Length |
| | thaliana | | NP_193041 | | |
| fatA | Brassica juncea | thioesterase | CAC39106 | 3.1.2.14 | C18:1 Chain Length |
| fatA | Cuphea hookeriana | thioesterase | AAC72883 | 3.1.2.14 | C18:1 Chain Length |
| tesA | Photbacterium | thioesterase | YP_130990 | 3.1.2.14 | Chain Length |
| | profundum | | | | |
| tesB | E. coli | thioesterase | NP_414986 | 3.1.2.14 | Chain Length |
| fadM | E. coli | thioesterase | NP_414977 | 3.1.2.14 | Chain Length |
| yciA | E. coli | thioesterase | NP_415769 | 3.1.2.14 | Chain Length |
| ybgC | E. coli | thioesterase | NP_415264 | 3.1.2.14 | Chain Length |

Saturation Level Control*

| | | | | | |
|---|---|---|---|---|---|
| Sfa | E. coli | suppressor of fabA | AAN79592, | none | increase |
| | | | AAC44390 | | monounsaturated |
| | | | | | fatty acids |
| fabA | E. coli K12 | β- | NP_415474 | 4.2.1.60 | produce unsaturated |
| | | hydroxydecanoylthioesterdehydratase/ | | | fatty acids |
| | | isomerase | | | |
| GnsA | E. coli | suppressors of the | ABD18647.1 | none | increase unsaturated |
| | | secG null mutation | | | fatty acid esters |
| GnsB | E. coli | suppressors of the | AAC74076.1 | none | increase unsaturated |
| | | secG null mutation | | | fatty acid esters |
| fabB | E. coli | 3-oxoacyl-[acyl- | BAA16180 | 2.3.1.41 | modulate |
| | | carrier-protein] | | | unsaturated fatty |
| | | synthase I | | | acid production |
| des | Bacillus subtilis | D5 fatty acyl | O34653 | 1.14.19 | modulate |
| | | desaturase | | | unsaturated fatty |
| | | | | | acid production |

Product Output: Ester Production

| | | | | | |
|---|---|---|---|---|---|
| AT3G51970 | Arabidopsis | long-chain-alcohol O- | NP_190765 | 2.3.1.26 | wax production |
| | thaliana | fatty-acyltransferase | | | |
| ELO1 | Pichia angusta | fatty acid elongase | BAD98251 | 2.3.1.— | produce very long |
| | | | | | chain length fatty |
| | | | | | acids |
| plsC | Saccharomyces | acyltransferase | AAA16514 | 2.3.1.51 | wax production |
| | cerevisiae | | | | |
| DAGAT/DGAT | Arabidopsis | diacylglycerolacyltransferase | AAF19262 | 2.3.1.20 | wax production |
| | thaliana | | | | |
| hWS | Homo sapiens | acyl-CoA wax alcohol | AAX48018 | 2.3.1.20 | wax production |
| | | acyltransferase | | | |
| aft1 | Acinetobacter | bifunctional wax ester | AAO17391 | 2.3.1.20 | wax production |
| | sp. ADP1 | synthase/acyl- | | | |
| | | CoA:diacylglycerolacyltransferase | | | |
| ES9 | Marinobacter | wax ester synthase | ABO21021 | 2.3.1.20 | wax production |
| | hydrocarbonoclasticus | | | | |
| mWS | Simmondsiachinensis | wax ester synthase | AAD38041 | 2.3.1.— | wax production |
| acr1 | Acinetobacter | acyl-CoA reductase | YP_047869 | 1.2.1.42 | modify output |
| | sp. ADP1 | | | | |
| yqhD | E. Coli K12 | alcohol dehydrogenase | AP_003562 | 1.1.—.— | modify output |
| AAT | Fragaria x | alcohol O- | AAG13130 | 2.3.1.84 | modify output |
| | ananassa | acetyltransferase | | | |

Product Output: Fatty Alcohol Output

| | | | | | |
|---|---|---|---|---|---|
| | | thioesterases (see | | | increase fatty |
| | | above) | | | acid/fatty alcohol |
| | | | | | production |
| BmFAR | Bombyxmori | FAR (fatty alcohol | BAC79425 | 1.1.1.— | convert acyl-CoA to |
| | | forming acyl-CoA | | | fatty alcohol |
| | | reductase) | | | |
| acr1 | Acinetobacter | acyl-CoA reductase | YP_047869 | 1.2.1.42 | reduce fatty acyl- |
| | sp. ADP1 | | | | CoA to fatty |
| | | | | | aldehydes |
| yqhD | E. coli W3110 | alcohol dehydrogenase | AP_003562 | 1.1.—.— | reduce fatty |
| | | | | | aldehydes to fatty |
| | | | | | alcohols; increase |
| | | | | | fatty alcohol |
| | | | | | production |
| alrA | Acinetobacter | alcohol dehydrogenase | CAG70252 | 1.1.—.— | reduce fatty |
| | sp. ADP1 | | | | aldehydes to fatty |
| | | | | | alcohols |
| BmFAR | Bombyxmori | FAR (fatty alcohol | BAC79425 | 1.1.1.— | reduce fatty acyl- |
| | | forming acyl-CoA | | | CoA to fatty alcohol |
| | | reductase) | | | |
| GTNG_1865 | Geobacillusther | long-chain aldehyde | YP_001125970 | 1.2.1.3 | reduce fatty |
| | modenitrificans | dehydrogenase | | | aldehydes to fatty |
| | NG80-2 | | | | alcohols |
| AAR | Synechococcus | acyl-ACP reductase | YP_400611 | 1.2.1.80 | reduce fatty acyl- |
| | elongatus | | | 1.2.1.42 | ACP/CoA to fatty |
| | | | | | aldehydes |
| carB | Mycobacterium | carboxylic acid | YP_889972 | 6.2.1.3, | reduce fatty acids to |
| | smegmatis | reductase (CAR) | | 1.2.1.42 | fatty aldehyde |
| | | protein | | | |
| FadD | E. coli K12 | acyl-CoA synthetase | NP_416319 | 6.2.1.3 | activates fatty acids |
| | | | | | to fatty acyl-CoAs |
| atoB | Erwinia carotovora | acetyl-CoA | YP_049388 | 2.3.1.9 | production of |
| | | acetyltransferase | | | butanol |
| hbd | Butyrivibrio fibrisolvens | beta-hydroxybutyryl- | BAD51424 | 1.1.1.157 | production of |
| | | CoA dehydrogenase | | | butanol |
| CPE0095 | Clostridium | crotonasebutyryl-CoA | BAB79801 | 4.2.1.55 | production of |
| | perfringens | dehydryogenase | | | butanol |
| bcd | Clostridium | butyryl-CoA | AAM14583 | 1.3.99.2 | production of |
| | beijerinckii | dehydryogenase | | | butanol |
| ALDH | Clostridium | coenzyme A-acylating | AAT66436 | 1.2.1.3 | production of |
| | beijerinckii | aldehyde | | | butanol |
| | | dehydrogenase | | | |
| AdhE | E. coli CFT073 | aldehyde-alcohol | AAN80172 | 1.1.1.1 | production of |
| | | dehydrogenase | | 1.2.1.10 | butanol |

Product Export

| | | | | | |
|---|---|---|---|---|---|
| AtMRP5 | Arabidopsis | Arabidopsis thaliana | NP_171908 | none | modify product |
| | thaliana | multidrug resistance- | | | export amount |
| | | associated | | | |
| AmiS2 | Rhodococcus | ABC transporter | JC5491 | none | modify product |
| | sp. | AmiS2 | | | export amount |
| AtPGP1 | Arabidopsis | Arabidopsis thaliana p | NP_181228 | none | modify product |
| | thaliana | glycoprotein 1 | | | export amount |
| AcrA | Candidatus | putative multidrug- | CAF23274 | none | modify product |
| | Protochlamydia | efflux transport protein | | | export amount |
| | amoebophila UWE25 | acrA | | | |
| AcrB | Candidatus | probable multidrug- | CAF23275 | none | modify product |
| | Protochlamydia | efflux transport | | | export amount |
| | amoebophila UWE25 | protein, acrB | | | |
| TolC | Francisella tularensis | outer membrane | ABD59001 | none | modify product |
| | subsp. | protein [Cell envelope | | | export amount |
| | novicida | biogenesis, | | | |
| AcrE | Shigella sonnei | transmembrane protein | YP_312213 | none | modify product |
| | Ss046 | affects septum | | | export amount |
| | | formation and cell | | | |
| | | membrane | | | |
| | | permeability | | | |
| AcrF | E. coli | acriflavine resistance | P24181 | none | modify product |
| | | protein F | | | export amount |
| tll1619 | Thermosynechococcus | multidrug efflux | NP_682409.1 | none | modify product |
| | elongatus [BP-1] | transporter | | | export amount |
| tll0139 | Thermosynechococcus | multidrug efflux | NP_680930.1 | none | modify product |
| | elongatus [BP-1] | transporter | | | export amount |

Fermentation

| | | | | | |
|---|---|---|---|---|---|
| replication | | | | | increase output |
| checkpoint | | | | | efficiency |
| genes | | | | | |
| umuD | Shigella sonnei | DNA polymerase V, | YP_310132 | 3.4.21.— | increase output |
| | Ss046 | subunit | | | efficiency |
| umuC | E. coli | DNA polymerase V, | ABC42261 | 2.7.7.7 | increase output |
| | | subunit | | | efficiency |
| pntA, pntB | Shigella flexneri | NADH:NADPH | P07001, | 1.6.1.2 | increase output |
| | | transhydrogenase | P0AB70 | | efficiency |
| | | (alpha and beta | | | |
| | | subunits) | | | |

Other

| | | | | | |
|---|---|---|---|---|---|
| fabK | Streptococcus | trans-2-enoyl-ACP | AAF98273 | 1.3.1.9 | Contributes to fatty |
| | pneumoniae | reductase II | | | acid biosynthesis |
| fabL | Bacillus | enoyl-(acyl carrier | AAU39821 | 1.3.1.9 | Contributes to fatty |
| | licheniformis | protein) reductase | | | acid biosynthesis |
| | DSM 13 | | | | |
| fabM | Streptococcus | trans-2, cis-3- | DAA05501 | 4.2.1.17 | Contributes to fatty |
| | mutans | decenoyl-ACP | | | acid biosynthesis |
| | | isomerase | | | |

Production of Fatty Acids

The recombinant host cells may include one or more polynucleotide sequences that encompass an open reading frame encoding an ACP and a thioesterase of EC 3.1.1.5 or EC 3.1.2.- (e.g., EC 3.1.2.14), together with operably-linked regulatory sequences that facilitate expression of the protein in the recombinant host cells in order to produce fatty acids. In the recombinant host cells, the open reading frame coding sequences and/or the regulatory sequences are modified relative to the corresponding wild-type gene encoding the thioesterase and/or ACP. The activity of the thioesterase in the recombinant host cell is modified relative to the activity of the thioesterase expressed from the corresponding wild-type gene in a corresponding host cell. In some embodiments, a fatty acid derivative composition comprising fatty acids is produced by culturing a recombinant cell in the presence of a carbon source under conditions effective to express the thioesterase. In related embodiments, the recombinant host cell includes a polynucleotide encoding a polypeptide having thioesterase activity; a polynucleotide encoding an ACP polypeptide; and optionally one or more additional polynucleotides encoding polypeptides having other fatty acid biosynthetic enzyme activities. In some such instances, the fatty acid produced by the action of the thioesterase is converted by one or more enzymes having a different fatty acid biosynthetic enzyme activity to another fatty acid derivative, such as, for example, a fatty ester, fatty aldehyde, fatty alcohol, or a hydrocarbon.

The chain length of a fatty acid, or a fatty acid derivative made therefrom, can be selected for by modifying the expression of particular thioesterases. The particular thioesterase will influence the chain length of fatty acid derivatives produced. The chain length of a fatty acid derivative substrate can be selected for by modifying the expression of selected thioesterases (e.g., EC 3.1.2.14 or EC 3.1.1.5). Thus, host cells can be engineered to express, overexpress, have attenuated expression, or not at all express one or more selected thioesterases to increase the production of a preferred fatty acid derivative substrate. For example, C₁₀ fatty acids can be produced by expressing a particular thioesterase that has a preference for producing C₁₀ fatty acids and attenuating thioesterases that have a preference for producing fatty acids other than C₁₀ fatty acids (e.g., a thioesterase which prefers to produce C₁₄ fatty acids). This would result in a relatively homogeneous population of fatty acids that have a carbon chain length of 10. In other instances, C₁₄ fatty acids can be produced by attenuating endogenous thioesterases that produce non-C₁₄ fatty acids and expressing the thioesterases that use C₁₄-ACP. In some situations, C₁₂ fatty acids can be produced by expressing thioesterases that use C₁₂-ACP and attenuating thioesterases that produce non-C₁₂ fatty acids. For example, C₁₂ fatty acids can be produced by expressing a thioesterase that has a preference for producing C₁₂ fatty acids and attenuating thioesterases that have a preference for producing fatty acids other than C₁₂ fatty acids. This would result in a relatively homogeneous population of fatty acids that have a carbon chain length of 12. In one preferred embodiment, the fatty acid composition is recovered from the extracellular environment of the recombinant host cells, i.e., the cell culture medium. In another embodiment, the fatty acid composition is recovered from the intracellular environment of the recombinant host cells. The fatty acid derivative composition produced by a recombinant host cell can be analyzed using methods known in the art, for example, GC-FID, in order to determine the distribution of particular fatty acid derivatives as well as chain lengths and degree of saturation of the components of the fatty acid derivative composition. Acetyl-CoA, malonyl-CoA, and fatty acid overproduction can be verified using methods known in the art, for example, by using radioactive precursors, HPLC, or GC-MS subsequent to cell lysis. Additional examples of thioesterases and polynucleotides encoding them for use in the fatty acid pathway are provided in PCT Publication No. WO 2010/075483, expressly incorporated by reference herein.

Production of Fatty Aldehydes

The recombinant host cells may include one or more polynucleotide sequences that encompass an open reading frame encoding an ACP and one or more biosynthetic proteins such as an acyl-ACP reductase (AAR) of EC 1.2.1.42 or 1.2.1.80; or a carboxylic acid reductase (CAR) of EC 6.2.1.3 or EC 1.2.1.42, together with operably-linked regulatory sequences that facilitate expression of the protein in the recombinant host cells in order to produce fatty aldehydes. In the recombinant host cells, the open reading frame coding sequences and/or the regulatory sequences are modified relative to the corresponding wild-type gene encoding the AAR or CAR and/or ACP. The recombinant host cell may also include one or more polynucleotide sequences that encompass an open reading frame encoding an ACP and one or more biosynthetic proteins such as an acyl-CoA reductase of EC 1.2.1.42 in combination with a thioesterase of EC 3.1.1.5 or EC 3.1.2.- (e.g., EC 3.1.2.14) and an acyl-CoA synthetase (FadD) of 6.2.1.3.

In some embodiments, a fatty acid produced by the recombinant host cell is converted into a fatty aldehyde. In some embodiments, the fatty aldehyde produced by the recombinant host cell is then converted into a fatty alcohol or a hydrocarbon. In some embodiments, native (endogenous) fatty aldehyde biosynthetic polypeptides, such as aldehyde reductases or alcohol dehydrogenases are present in the host cell (e.g., E. coli) and are effective to convert fatty aldehydes to fatty alcohols. In other embodiments, a native (endogenous) fatty aldehyde biosynthetic polypeptide is overexpressed. In still other embodiments, an exogenous fatty aldehyde biosynthetic polypeptide is introduced into a recombinant host cell and expressed or overexpressed. A native or recombinant host cell may include a polynucleotide encoding an enzyme having fatty aldehyde biosynthesis activity (also referred to herein as a fatty aldehyde biosynthetic polypeptide or a fatty aldehyde biosynthetic polypeptide or enzyme). A fatty aldehyde is produced when the fatty aldehyde biosynthetic enzyme (e.g., AAR) is expressed or overexpressed in the host cell. A recombinant host cell engineered to produce a fatty aldehyde will typically convert some of the fatty aldehyde to a fatty alcohol.

In some embodiments, a fatty aldehyde is produced by expressing or overexpressing in the recombinant host cell a polynucleotide encoding a polypeptide having fatty aldehyde biosynthetic activity such as carboxylic acid reductase (CAR) activity or acyl-ACP reductase (AAR) activity. CarB, is an exemplary carboxylic acid reductase. In practicing the disclosure, a gene encoding a carboxylic acid reductase polypeptide may be expressed or overexpressed in the host cell (see FIG. 4). In some embodiments, the CarB polypeptide has the amino acid sequence of SEQ ID NO: 90. In other embodiments, the CarB polypeptide is encoded by SEQ ID NO: 88 (CarB) or SEQ ID NO: 89 (CarB60), or a mutant or variant thereof. Examples of carboxylic acid reductase (CAR) polypeptides and polynucleotides encoding them include, but are not limited to FadD9 (EC 6.2.1.-, UniProtKB Q50631, GenBank NP_{—}217106), CarA (GenBank ABK75684), CarB (GenBank YP889972) and related polypeptides described in PCT Publication No. WO 2010/042664 and U.S. Pat. No. 8,097,439, each of which is expressly incorporated by reference herein. In some embodiments the recombinant host cell further comprises a polynucleotide encoding a thioesterase.

In some embodiments, the fatty aldehyde is produced by expressing or overexpressing in the recombinant host cell a polynucleotide encoding a fatty aldehyde biosynthetic polypeptide, such as a polypeptide having acyl-ACP reductase (AAR) activity. Expression of AAR in a recombinant host cell results in the production of fatty aldehydes and/or fatty alcohols (FIG. 4). Exemplary AAR polypeptides are described in PCT Publication Nos. WO2009/140695 and WO/2009/140696, both of which are expressly incorporated by reference herein. A composition comprising a fatty aldehyde (a fatty aldehyde composition) is produced by culturing a host cell in the presence of a carbon source under conditions effective to express the fatty aldehyde biosynthetic enzyme. In some embodiments, the fatty aldehyde composition comprises fatty aldehydes and fatty alcohols. In one preferred embodiment, the fatty aldehyde composition is recovered from the extracellular environment of the recombinant host cells, i.e., the cell culture medium. In another embodiment, the fatty aldehyde composition is recovered from the intracellular environment of the recombinant host cells.

Production of Fatty Alcohols

The recombinant host cells may include one or more polynucleotide sequences that encompass an open reading frame encoding an ACP and one or more biosynthetic proteins such as an acyl-ACP reductase (AAR) of EC 1.2.1.42 or 1.2.1.80; or a carboxylic acid reductase (CAR) of EC 6.2.1.3 or EC 1.2.1.42 in combination with an endogenous or exogenous aldehyde reductase or alcohol dehydrogenase, together with operably-linked regulatory sequences that facilitate expression of the protein in the recombinant host cells in order to produce fatty alcohols. In the recombinant host cells, the open reading frame coding sequences and/or the regulatory sequences are modified relative to the corresponding wild-type gene encoding the AAR or CAR and optional aldehyde reductase or alcohol dehydrogenase and/or ACP.

In some embodiments, the recombinant host cell comprises a polynucleotide encoding a polypeptide (an enzyme) having fatty alcohol biosynthetic activity (also referred to herein as a fatty alcohol biosynthetic polypeptide or a fatty alcohol biosynthetic enzyme), and a fatty alcohol is produced by the recombinant host cell. A composition comprising fatty alcohols (a fatty alcohol composition) may be produced by culturing the recombinant host cell in the presence of a carbon source under conditions effective to express a fatty alcohol biosynthetic enzyme. Native (endogenous) aldehyde reductases or alcohol dehydrogenases present in a recombinant host cell (e.g., E. coli) will convert fatty aldehydes into fatty alcohols. In some embodiments, the fatty alcohol composition includes one or more fatty alcohols, however, a fatty alcohol composition may comprise other fatty acid derivatives. In one preferred embodiment, the fatty alcohol composition is recovered from the extracellular environment of the recombinant host cells, i.e., the cell culture medium. In another embodiment, the fatty alcohol composition is recovered from the intracellular environment of the recombinant host cells.

In one approach, recombinant host cells have been engineered to produce fatty alcohols by expressing a thioesterase, which catalyzes the conversion of acyl-ACPs into free fatty acids (FFAs) and a carboxylic acid reductase (CAR), which converts free fatty acids into fatty aldehydes. Native (endogenous) aldehyde reductases or alcohol dehydrogenases present in the host cell (e.g., E. coli) can convert the fatty aldehydes into fatty alcohols. In some embodiments, native (endogenous) fatty aldehyde biosynthetic polypeptides, such as aldehyde reductases and/or alcohol dehydrogenases present in the host cell, may be sufficient to convert fatty aldehydes to fatty alcohols. However, in other embodiments, a native (endogenous) fatty aldehyde biosynthetic polypeptide is overexpressed and in still other embodiments, an exogenous fatty aldehyde biosynthetic polypeptide is introduced into a recombinant host cell and expressed or overexpressed. In some embodiments, the fatty alcohol is produced by expressing or overexpressing in the recombinant host cell a polynucleotide encoding a polypeptide having fatty alcohol biosynthetic activity which converts a fatty aldehyde to a fatty alcohol. For example, an alcohol dehydrogenase or aldehyde reductase (e.g., EC 1.1.1.1), may be used in practicing the disclosure. As used herein, an alcohol dehydrogenase or aldehyde reductase refers to a polypeptide capable of catalyzing the conversion of a fatty aldehyde to an alcohol (e.g., a fatty alcohol). One of ordinary skill in the art will appreciate that certain alcohol dehydrogenases are capable of catalyzing other reactions as well, and these non-specific alcohol dehydrogenases also are encompassed by the term alcohol dehydrogenase. Examples of alcohol dehydrogenase polypeptides useful in accordance with the disclosure include, but are not limited to AlrA of Acinetobacter sp. M-1 (CAG70252) or AlrA homologs such as AlrAadp1, endogenous E. coli alcohol dehydrogenases such as YjgB, (AAC77226), DkgA (NP_{—}417485), DkgB (NP_{—}414743), YdjL (AAC74846), YdjJ (NP_{—}416288), AdhP (NP_{—}415995), YhdH (NP_{—}417719), YahK (NP_{—}414859), YphC (AAC75598), YqhD (446856) and YbbO [AAC73595.1]. Additional examples are described in International Patent Application Publication Nos. WO 2007/136762, WO2008/119082 and WO 2010/062480, each of which is expressly incorporated by reference herein. In certain embodiments, the fatty alcohol biosynthetic polypeptide has aldehyde reductase or alcohol dehydrogenase activity (EC 1.1.1.1).

In another approach, recombinant host cells have been engineered to produce fatty alcohols by expressing fatty alcohol forming acyl-CoA reductases or fatty acyl reductases (FARs) which convert fatty acyl-thioester substrates (e.g., fatty acyl-CoA or fatty acyl-ACP) to fatty alcohols. In some embodiments, the fatty alcohol is produced by expressing or overexpressing a polynucleotide encoding a polypeptide having fatty alcohol forming acyl-CoA reductase (FAR) activity in a recombinant host cell. Examples of FAR polypeptides useful in accordance with this embodiment are described in PCT Publication No. WO 2010/062480 which is expressly incorporated by reference herein. Fatty alcohol may be produced via an acyl-CoA dependent pathway utilizing fatty acyl-ACP and fatty acyl-CoA intermediates and an acyl-CoA independent pathway utilizing fatty acyl-ACP intermediates but not a fatty acyl-CoA intermediate. In particular embodiments, the enzyme encoded by the overexpressed gene includes, but is not limited to, a fatty acid synthase, an acyl-ACP thioesterase, a fatty acyl-CoA synthase and an acetyl-CoA carboxylase (ACC). In some embodiments, the protein encoded by the overexpressed gene is endogenous to the host cell. In other embodiments, the protein encoded by the overexpressed gene is heterologous or exogenous to the host cell.

Fatty alcohols are also made in nature by enzymes that are able to reduce various acyl-ACP or acyl-CoA molecules to the corresponding primary alcohols (see U.S. Patent Publication Nos. 20100105963 and 20110206630; and U.S. Pat. No. 8,097,439, expressly incorporated by reference herein). Strategies to increase production of fatty alcohols by recombinant host cells include increased flux through the fatty acid biosynthetic pathway by overexpression of native fatty acid biosynthetic genes and/or expression of exogenous fatty acid biosynthetic genes from different organisms in the production host such that fatty alcohol biosynthesis is increased.

Production of Esters

The recombinant host cells may include one or more polynucleotide sequences that encompass an open reading frame encoding an ACP and one or more biosynthetic proteins such as an ester synthase (ES) of EC 2.3.1.75; or an ES in combination with an endogenous or exogenous thioesterase (TE) of EC 3.1.1.5 or EC 3.1.2.- and acyl-CoA synthetase/synthase (fadD) of EC 6.2.1.3, together with operably-linked regulatory sequences that facilitate expression of the protein in the recombinant host cells in order to produce fatty esters (see FIG. 5). In the recombinant host cells, the open reading frame coding sequences and/or the regulatory sequences are modified relative to the corresponding wild-type gene encoding the ES and optional TE and fadD and/or ACP.

A fatty ester as referred to herein can be any ester made from a fatty acid, for example a fatty acid ester. In some embodiments, a fatty ester contains an A side and a B side. The A side of an ester refers to the carbon chain attached to the carboxylate oxygen of the ester. The B side of an ester refers to the carbon chain including the parent carboxylate of the ester. In embodiments where the fatty ester is derived from the fatty acid biosynthetic pathway, the A side is contributed by an alcohol, and the B side is contributed by a fatty acid. Any alcohol can be used to form the A side of the fatty esters. For example, the alcohol can be derived from the fatty acid biosynthetic pathway. Alternatively, the alcohol can be produced through non-fatty acid biosynthetic pathways. Moreover, the alcohol can be provided exogenously. For example, the alcohol can be supplied in the fermentation broth in instances where the fatty ester is produced by an organism. Alternatively, a carboxylic acid, such as a fatty acid or acetic acid, can be supplied exogenously in instances where the fatty ester is produced by an organism that can also produce alcohol. The carbon chains comprising the A side or B side can be of any length. In one embodiment, the A side of the ester is at least about 1, 2, 3, 4, 5, 6, 7, 8, 10, 12, 14, 16, or 18 carbons in length. When the fatty ester is a fatty acid methyl ester, the A side of the ester is 1 carbon in length. When the fatty ester is a fatty acid ethyl ester, the A side of the ester is 2 carbons in length. The B side of the ester can be at least about 4, 6, 8, 10, 12, 14, 16, 18, 20, 22, 24, or 26 carbons in length. The A side and/or the B side can be straight or branched chain. The branched chains can have one or more points of branching. In addition, the branched chains can include cyclic branches. Furthermore, the A side and/or B side can be saturated or unsaturated. If unsaturated, the A side and/or B side can have one or more points of unsaturation.

In one embodiment, the fatty ester is produced biosynthetically. In this embodiment, the fatty acid is first activated. Examples of activated fatty acids are acyl-CoA, acyl ACP, and acyl phosphate. Acyl-CoA can be a direct product of fatty acid biosynthesis or degradation. In addition, acyl-CoA can be synthesized from a free fatty acid, a CoA, and an adenosine nucleotide triphosphate (ATP). An example of an enzyme which produces acyl-CoA is acyl-CoA synthase. In some embodiments, the recombinant host cell comprises a polynucleotide encoding a polypeptide, e.g., an enzyme having ester synthase activity, (also referred to herein as an ester synthase polypeptide or an ester synthase). A fatty ester is produced by a reaction catalyzed by the ester synthase polypeptide expressed or overexpressed in the recombinant host cell. In some embodiments, a composition encompasses fatty esters (also referred to herein as a fatty ester composition) including fatty esters produced by culturing the recombinant cell in the presence of a carbon source under conditions effective to express an ester synthase. In some embodiments, the fatty ester composition is recovered from the cell culture. Ester synthase polypeptides include, for example, an ester synthase polypeptide classified as EC 2.3.1.75, or any other polypeptide which catalyzes the conversion of an acyl-thioester to a fatty ester, including, without limitation, a thioesterase, an ester synthase, an acyl-CoA:alcoholtransacylase, an acyltransferase, or a fatty acyl-CoA:fatty alcohol acyltransferase. For example, a polynucleotide expressed in the recombinant host cells may encode wax/dgat, a bifunctional ester synthase/acyl-CoA:diacylglycerol acyltransferase from Simmondsia chinensis, Acinetobacter sp. strain ADP, Alcanivorax borkumensis, Pseudomonas aeruginosa, Fundibacter jadensis, Arabidopsis thaliana, or Alkaligenes eutrophus. In a particular embodiment, the ester synthase polypeptide is an Acinetobacter sp. diacylglycerol O-acyltransferase (wax-dgat; UniProtKB Q8GGG1, GenBank AA017391) or Simmondsia chinensis wax synthase (UniProtKB Q9XGY6, GenBank AAD38041). In another embodiment, the ester synthase polypeptide is, for example, ES9, a wax ester synthase from Marinobacter hydrocarbonoclasticus, encoded by the ws2 gene (SEQ ID NO: 93); DSM 8798, UniProtKB A3RE51 (SEQ ID NO: 94); or ES8 of M. hydrocarbonoclasticus DSM8798 (GenBank Accession No. AB021020), encoded by the ws1 gene. In a particular embodiment, the polynucleotide encoding the ester synthase polypeptide is overexpressed in the recombinant host cell. In some embodiments, a fatty acid ester is produced by a recombinant host cell engineered to express three fatty acid biosynthetic enzymes including a thioesterase (TE) enzyme, an acyl-CoA synthetase (fadD) enzyme, and an ester synthase (ES) enzyme (see FIG. 5, the three enzyme system). In other embodiments, a fatty acid ester is produced by a recombinant host cell engineered to express one fatty acid biosynthetic enzyme, an ester synthase (ES) enzyme (see FIG. 5, the one enzyme system). Examples of ester synthase polypeptides (and polynucleotides encoding them) suitable for use in these embodiments include those described in PCT Publication Nos. WO 2007/136762, WO2008/119082, and WO/2011/038134 (three enzyme system) and WO/2011/038132 (one enzyme system), each of which is expressly incorporated by reference herein. The recombinant host cell may produce a fatty ester, such as a fatty acid methyl ester, a fatty acid ethyl ester and/or a wax ester. In one preferred embodiment, the ester composition is recovered from the extracellular environment of the recombinant host cells, i.e., the cell culture medium. In another embodiment, the ester composition is recovered from the intracellular environment of the recombinant host cells.

Production of Hydrocarbons

The recombinant host cells may include one or more polynucleotide sequences that encompass an open reading frame encoding an ACP and one or more biosynthetic proteins such as an acyl-ACP reductase (AAR) of EC 1.2.1.42 or 1.2.1.80 in combination with an endogenous or exogenous decarbonylase (ADC); or an endogenous or exogenous thioesterase (TE) of EC 3.1.1.5 or EC 3.1.2.- in combination with a decarboxylase together with operably-linked regulatory sequences that facilitate expression of the protein in the recombinant host cells in order to produce hydrocarbons (e.g., alkanes, olefins) and or ketones. In the recombinant host cells, the open reading frame coding sequences and/or the regulatory sequences are modified relative to the corresponding wild-type gene encoding the AAR and ADC or TE and decarboxylase and/or ACP.

Thus, this aspect is based, at least in part, on the discovery that altering the level of expression of a fatty aldehyde biosynthetic polypeptide such as an AAR and a hydrocarbon biosynthetic polypeptide such as a decarbonylase polypeptide in a recombinant host cell facilitates enhanced production of hydrocarbons by the cell. In one embodiment, the recombinant host cell produces a hydrocarbon, such as an alkane or an alkene. In some embodiments, a fatty aldehyde produced by a recombinant host cell is converted by decarbonylation, removing a carbon atom to form a hydrocarbon. In other embodiments, a fatty acid produced by a recombinant host cell is converted by decarboxylation, removing a carbon atom to form a terminal olefin. In some embodiments, an acyl-ACP intermediate is converted by decarboxylation, removing a carbon atom to form an internal olefin or a ketone (see FIG. 6). In some embodiments, the recombinant host cell includes a polynucleotide encoding a polypeptide (an enzyme) having hydrocarbon biosynthetic activity (also referred to herein as a hydrocarbon biosynthetic polypeptide or a hydrocarbon biosynthetic enzyme), and the hydrocarbon is produced by expression or overexpression of the hydrocarbon biosynthetic enzyme in a recombinant host cell. An alkane biosynthetic pathway encompassing an acyl-ACP reductase (AAR) and an aldehyde decarbonylase (ADC) of EC 4.1.99.5, which together convert intermediates of fatty acid metabolism to alkanes and alkenes, has been used to engineer recombinant host cells for the production of hydrocarbons (see U.S. Pat. No. 8,323,924, which is expressly incorporated by reference herein).

In some embodiments, a composition that includes hydrocarbons (also referred to herein as a hydrocarbon composition) is produced by culturing the recombinant cell in the presence of a carbon source under conditions effective to express the AAR and ADC polynucleotides. In some embodiments, the hydrocarbon composition includes saturated and unsaturated hydrocarbons, however, a hydrocarbon composition may include other fatty acid derivatives. In one preferred embodiment, the hydrocarbon composition is recovered from the extracellular environment of the recombinant host cells, i.e., the cell culture medium. In another embodiment, the hydrocarbon composition is recovered from the intracellular environment of the recombinant host cells. A hydrocarbon such as an alkane refers to a saturated hydrocarbon or compound that is made of carbon (C) and hydrogen (H), wherein these atoms are linked together by single bonds (i.e., they are saturated compounds). An olefin and an alkene refer to the same type of hydrocarbon (compound) containing at least one carbon-to-carbon double bond (i.e., an unsaturated compound). Examples of alkenes/olefins are terminal olefins (also called α-olefins, terminal alkenes, or 1-alkenes) that have the chemical formula CₓH₂ₓ, which is different from other olefins with a similar molecular formula distinguished by linearity of the hydrocarbon chain and the position of the double bond at the primary or alpha position. In some embodiments, a terminal olefin is produced by expressing or overexpressing in the recombinant host cell a polynucleotide encoding a hydrocarbon biosynthetic polypeptide, such as a polypeptide having decarboxylase activity as described, for example, in PCT Publication No. WO 2009/085278, which is expressly incorporated by reference herein. In some embodiments the recombinant host cell further includes a polynucleotide encoding a thioesterase.

In other embodiments, a ketone is produced by expressing or overexpressing in the recombinant host cell a polynucleotide encoding a hydrocarbon biosynthetic polypeptide, such as a polypeptide having OleA activity as described, for example, in PCT Publication No. WO 2008/147781, which is expressly incorporated by reference herein. In related embodiments, an internal olefin is produced by expressing or overexpressing in the recombinant host cell a polynucleotide encoding a hydrocarbon biosynthetic polypeptide, such as a polypeptide having OleCD or OleBCD activity together with a polypeptide having OleA activity as described, for example, in PCT Publication No. WO 2008/147781, which is expressly incorporated by reference herein.

Recombinant Host Cells and Cell Cultures

Strategies to increase production of fatty acid derivatives by recombinant host cells include increased flux through the fatty acid biosynthetic pathway by overexpression of native fatty acid biosynthetic genes and expression of exogenous fatty acid biosynthetic genes from different organisms in the production host as described above (supra). A recombinant host cell (or engineered host cell) refers to a host cell whose genetic makeup has been altered relative to the corresponding wild-type host cell, for example, by deliberate introduction of new genetic elements and/or deliberate modification of genetic elements naturally present in the host cell. The offspring of such recombinant host cells also contain these new and/or modified genetic elements. In any of the aspects of the disclosure described herein, the host cell can be selected from a plant cell, an insect cell, a fungus cell (e.g., a filamentous fungus, such as Candida sp., or a budding yeast, such as Saccharomyces sp.), an algal cell, and a bacterial cell. In one preferred embodiment, recombinant host cells are recombinant microorganisms that are derived from bacteria. In another embodiment, recombinant host cells are recombinant microorganisms that are derived from fungus. In yet another embodiment, recombinant host cells are recombinant microorganisms that are derived from algae. In yet another embodiment, recombinant host cells are recombinant microorganisms that are derived from plants or insects.

Examples of host cells that are microorganisms include, but are not limited to, cells from the genus Escherichia, Bacillus, Lactobacillus, Zymomonas, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces. In some embodiments, the host cell is a Gram-positive bacterial cell. In other embodiments, the host cell is a Gram-negative bacterial cell. In one preferred embodiment, the host cell is an E. coli cell. In other embodiments, the host cell is a Bacillus lentus cell, a Bacillus brevis cell, a Bacillus stearothermophilus cell, a Bacillus lichenoformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell. In other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillusoryzae cell, a Humicolainsolens cell, a Humicola lanuginose cell, a Rhodococcusopacus cell, a Rhizomucormiehei cell, or a Mucormichei cell. In yet other embodiments, the host cell is a Streptomyces lividans cell or a Streptomyces murinus cell. In yet other embodiments, the host cell is an Actinomycetes cell. In some embodiments, the host cell is a Saccharomyces cerevisiae cell. In other embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Arabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridium thermocellum, Penicillium chrysogenum, Pichiapastoris, Saccharomyces cerevisiae, Schizosaccharomyces pombe, Pseudomonas fluorescens, or Zymomonas mobilis.

A large variety of fatty acid derivatives can be produced by recombinant host cells and the strain improvements described herein, including, but not limited to, fatty acids, acyl-CoA, fatty aldehydes, short chain alcohols, fatty alcohols, hydrocarbons (e.g., alkanes, alkenes or olefins, such as terminal or internal olefins), esters such as wax esters, or fatty acid esters (e.g., fatty acid methyl esters (FAME) or fatty acid ethyl esters (FAEE)), and ketones. In some embodiments of the present disclosure, the higher titer of fatty acid derivatives in a particular composition is a higher titer of a particular type of fatty acid derivative (e.g., fatty alcohols, fatty acid esters, or hydrocarbons) produced by a recombinant host cell culture relative to the titer of the same fatty acid derivatives produced by a control culture of a corresponding wild-type host cell. In such cases, the fatty acid derivative compositions may include, for example, a mixture of the fatty alcohols with a variety of chain lengths and saturation or branching characteristics. In other embodiments of the present disclosure, the higher titer of fatty acid derivatives in a particular compositions is a higher titer of a combination of different fatty acid derivatives (for example, fatty aldehydes and alcohols, or fatty acids and esters) relative to the titer of the same fatty acid derivative produced by a control culture of a corresponding wild-type host cell.

Engineering Host Cells

In some embodiments, a polynucleotide (or gene) sequence is provided to the host cell by way of a recombinant vector, which includes a promoter operably linked to the polynucleotide sequence. In certain embodiments, the promoter is a developmentally-regulated, an organelle-specific, a tissue-specific, an inducible, a constitutive, or a cell-specific promoter. In some embodiments, the recombinant vector includes at least one sequence selected from an expression control sequence operatively coupled to the polynucleotide sequence; a selection marker operatively coupled to the polynucleotide sequence; a marker sequence operatively coupled to the polynucleotide sequence; a purification moiety operatively coupled to the polynucleotide sequence; a secretion sequence operatively coupled to the polynucleotide sequence; and a targeting sequence operatively coupled to the polynucleotide sequence. The expression vectors described herein include a polynucleotide sequence in a form suitable for expression of the polynucleotide sequence in a host cell. It will be appreciated by those skilled in the art that the design of the expression vector can depend on such factors as the choice of the host cell to be transformed, the level of expression of polypeptide desired, and the like. The expression vectors described herein can be introduced into host cells to produce polypeptides, including fusion polypeptides, encoded by the polynucleotide sequences as described above (supra). Expression of genes encoding polypeptides in prokaryotes, for example, E. coli, is most often carried out with vectors containing constitutive or inducible promoters directing the expression of either fusion or non-fusion polypeptides. Fusion vectors add a number of amino acids to a polypeptide encoded therein, usually to the amino- or carboxy-terminus of the recombinant polypeptide. Such fusion vectors typically serve one or more of the following three purposes, including to increase expression of the recombinant polypeptide; to increase the solubility of the recombinant polypeptide; and to aid in the purification of the recombinant polypeptide by acting as a ligand in affinity purification. Often, in fusion expression vectors, a proteolytic cleavage site is introduced at the junction of the fusion moiety and the recombinant polypeptide. This enables separation of the recombinant polypeptide from the fusion moiety after purification of the fusion polypeptide. Examples of such enzymes, and their cognate recognition sequences, include Factor Xa, thrombin, and enterokinase. Exemplary fusion expression vectors include pGEX vector (Pharmacia Biotech, Inc., Piscataway, N.J.; Smith et al. (1988) Gene 67:31-40), pMAL vector (New England Biolabs, Beverly, Mass.), and pRITS vector (Pharmacia Biotech, Inc., Piscataway, N.J.), which fuse glutathione S-transferase (GST), maltose E binding protein, or protein A, respectively, to the target recombinant polypeptide.

Examples of inducible, non-fusion E. coli expression vectors include pTrc vector (Amann et al. (1988) Gene 69:301-315) and pET 11d vector (Studier et al., Gene Expression Technology: Methods in Enzymology 185, Academic Press, San Diego, Calif. (1990) 60-89). Target gene expression from the pTrc vector relies on host RNA polymerase transcription from a hybrid trp-lac fusion promoter. Target gene expression from the pET 11d vector relies on transcription from a T7 gn10-lac fusion promoter mediated by a coexpressed viral RNA polymerase (T7 gn1). This viral polymerase is supplied by host strains such as BL21(DE3) or HMS174(DE3) from a resident λ prophage harboring a T7 gn1 gene under the transcriptional control of the lacUV 5 promoter. Suitable expression systems for both prokaryotic and eukaryotic cells are well known in the art (see, e.g., Sambrook et al. (1989) Molecular Cloning: A Laboratory Manual, second edition, Cold Spring Harbor Laboratory). Examples of inducible, non-fusion E. coli expression vectors include pTrc vector (Amann et al. (1988) Gene 69:301-315) and PET 11d vector (Studier et al. (1990) Gene Expression Technology: Methods in Enzymology 185, Academic Press, San Diego, Calif., pp. 60-89). In certain embodiments, a polynucleotide sequence of the disclosure is operably linked to a promoter derived from bacteriophage T5. In one embodiment, the host cell is a yeast cell. In this embodiment, the expression vector is a yeast expression vector. Vectors can be introduced into prokaryotic or eukaryotic cells via a variety of art-recognized techniques for introducing foreign nucleic acid (e.g., DNA) into a host cell. Suitable methods for transforming or transfecting host cells can be found in, for example, Sambrook et al. (supra). For stable transformation of bacterial cells, it is known that, depending upon the expression vector and transformation technique used, a certain fraction of cells will take-up and replicate the expression vector. In order to identify and select these transformants, a gene that encodes a selectable marker (e.g., resistance to an antibiotic) can be introduced into the host cells along with the gene of interest. Selectable markers include those that confer resistance to drugs such as, but not limited to, ampicillin, kanamycin, chloramphenicol, or tetracycline. Nucleic acids encoding a selectable marker can be introduced into a host cell on the same vector as that encoding a polypeptide described herein or can be introduced on a separate vector. Cells stably transformed with the introduced nucleic acid can be identified by growth in the presence of an appropriate selection drug. The engineered or recombinant host cell as described herein (supra) is a cell used to produce a fatty acid derivative composition. In any of the aspects of the disclosure described herein, the host cell can be selected from a eukaryotic plant, bacteria, algae, cyanobacterium, green-sulfur bacterium, green non-sulfur bacterium, purple sulfur bacterium, purple non-sulfur bacterium, extremophile, yeast, fungus, engineered organisms thereof, or a synthetic organism. In some embodiments, the host cell is light dependent or fixes carbon. In some embodiments, the host cell has autotrophic activity. Various host cells can be used to produce fatty acid derivatives, as described herein.

The host cells or microorganisms of the disclosure include host strains or host cells that are genetically engineered to contain alterations in order to test the efficiency of specific mutations on enzymatic activities (i.e., recombinant cells or microorganisms). Various optional genetic manipulations and alterations can be used interchangeably from one host cell to another, depending on what native enzymatic pathways are present in the original host cell. In one embodiment, a host strain can be used for testing the expression of an ACP polypeptide in combination with other biosynthetic polypeptides (e.g., enzymes). A host strain may encompasses a number of genetic alterations in order to test specific variables, including but not limited to, culture conditions including fermentation components, carbon source (e.g., feedstock), temperature, pressure, reduced culture contamination conditions, and oxygen levels.

In one embodiment, a host strain encompasses an optional fadE and fhuA deletion. Acyl-CoA dehydrogenase (FadE) is an enzyme that is important for metabolizing fatty acids. It catalyzes the second step in fatty acid utilization (beta-oxidation), which is the process of breaking long chains of fatty acids (acyl-CoAs) into acetyl-CoA molecules. More specifically, the second step of the β-oxidation cycle of fatty acid degradation in bacteria is the oxidation of acyl-CoA to 2-enoyl-CoA, which is catalyzed by FadE. When E. coli lacks FadE, it cannot grow on fatty acids as a carbon source but it can grow on acetate. The inability to utilize fatty acids of any chain length is consistent with the reported phenotype of fadE strains, i.e., fadE mutant strains where FadE function is disrupted. The fadE gene can be optionally knocked out or attenuated to assure that acyl-CoAs, which may be intermediates in a fatty acid derivative pathway, can accumulate in the cell such that all acyl-CoAs can be efficiently converted to fatty acid derivatives. However, fadE attenuation is optional when sugar is used as a carbon source since under such condition expression of FadE is likely repressed and FadE therefore may only be present in small amounts and not able to efficiently compete with ester synthase or other enzymes for acyl-CoA substrates. FadE is repressed due to catabolite repression. E. coli and many other microbes prefer to consume sugar over fatty acids, so when both sources are available sugar is consumed first by repressing the fad regulon (see D. Clark, J Bacteriol. (1981) 148(2):521-6)). Moreover, the absence of sugars induces FadE expression. Acyl-CoA intermediates could be lost to the beta oxidation pathway since the proteins expressed by the fad regulon (including FadE) are up-regulated and will efficiently compete for acyl-CoAs. Thus, it can be beneficial to have the fadE gene knocked out or attenuated. Since most carbon sources are mainly sugar based, it is optional to attenuate FadE. The gene fhuA codes for the TonA protein, which is an energy-coupled transporter and receptor in the outer membrane of E. coli (V. Braun (2009) J Bacteriol. 191(11):3431-3436). Its deletion is optional. The fhuA deletion allows the cell to become more resistant to phage attack which can be beneficial in certain fermentation conditions. Thus, it may be desirable to delete fhuA in a host cell that is likely subject to potential contamination during fermentation runs.

In another embodiment, the host strain (supra) also encompasses optional overexpression of one or more of the following genes including fadR, fabA, fabD, fabG, fabH, fabV, and/or fabF. Examples of such genes are fadR from Escherichia coli, fabA from Salmonella typhimurium (NP_{—}460041), fabD from Salmonella typhimurium (NP_{—}460164), fabG from Salmonella typhimurium (NP_{—}460165), fabH from Salmonella typhimurium (NP_{—}460163), fabV from Vibrio cholera (YP_{—}001217283), and fabF from Clostridium acetobutylicum (NP_{—}350156). The overexpression of one or more of these genes, which code for enzymes and regulators in fatty acid biosynthesis, can serve to increase the titer of fatty-acid derivative compounds under various culture conditions.

In another embodiment, E. coli strains are used as host cells for the production of fatty acid derivatives. Similarly, these host cells provide optional overexpression of one or more biosynthesis genes (i.e., genes coding for enzymes and regulators of fatty acid biosynthesis) that can further increase or enhance the titer of fatty-acid derivative compounds such as fatty acid derivatives (e.g., fatty acids, fatty esters, fatty alcohols, fatty aldehydes, hydrocarbons, etc.) under various culture conditions including, but not limited to, fadR, fabA, fabD, fabG, fabH, fabV and/or fabF. Examples of genetic alterations include fadR from Escherichia coli, fabA from Salmonella typhimurium (NP_{—}460041), fabD from Salmonella typhimurium (NP_{—}460164), fabG from Salmonella typhimurium (NP_{—}460165), fabH from Salmonella typhimurium (NP_{—}460163), fabV from Vibrio cholera (YP_{—}001217283), and fabF from Clostridium acetobutylicum (NP_{—}350156). In some embodiments, synthetic operons that carry these biosynthetic genes can be engineered and expressed in cells in order to test fatty acid derivative overexpression under various culture conditions and/or further enhance fatty acid derivative production. Such synthetic operons contain one or more biosynthetic gene. The ifab138 operon, for example, is an engineered operon that contains optional fatty acid biosynthetic genes, including fabV from Vibrio cholera, fabH from Salmonella typhimurium, fabD from S. typhimurium, fabG from S. typhimurium, fabA from S. typhimurium and/or fabF from Clostridium acetobutylicum that can be used to facilitate overexpression of fatty acid derivatives in order to test specific culture conditions. One advantage of such synthetic operons is that the rate of fatty acid derivative production can be further increased or enhanced.

In some embodiments, the host cells or microorganisms that are used to express ACP and other biosynthetic enzymes (e.g., TE, ES, CAR, AAR, ADC, etc.) will further express genes that encompass certain enzymatic activities that can increase the production to one or more particular fatty acid derivative(s) such as fatty esters, fatty alcohols, fatty amines, fatty aldehydes, bifunctional fatty acid derivatives, diacids and the like. In one embodiment, the host cell has thioesterase activity (E.C. 3.1.2.* or E.C. 3.1. 2.14 or E.C. 3.1.1.5) for the production of fatty acids which can be increased by overexpressing the gene. In another embodiment, the host cell has ester synthase activity (E.C. 2.3.1.75) for the production of fatty esters. In another embodiment, the host cell has acyl-ACP reductase (AAR) (E.C. 1.2.1.80) activity and/or alcohol dehydrogenase activity (E.C. 1.1.1.1.) and/or fatty alcohol acyl-CoA reductase (FAR) (E.C. 1.1.1.*) activity and/or carboxylic acid reductase (CAR) (EC 1.2.99.6) activity for the production of fatty alcohols. In another embodiment, the host cell has acyl-ACP reductase (AAR) (E.C. 1.2.1.80) activity for the production of fatty aldehydes. In another embodiment, the host cell has acyl-ACP reductase (AAR) (E.C. 1.2.1.80) activity and decarbonylase (ADC) activity for the production of alkanes and alkenes. In another embodiment, the host cell has acyl-CoA reductase (E.C. 1.2.1.50) activity, acyl-CoA synthase (FadD) (E.C. 2.3.1.86) activity, and thioesterase (E.C. 3.1.2.* or E.C. 3.1. 2.14 or E.C. 3.1.1.5) activity for the production of fatty alcohols. In another embodiment, the host cell has ester synthase activity (E.C. 2.3.1.75), acyl-CoA synthase (FadD) (E.C. 2.3.1.86) activity, and thioesterase (E.C. 3.1.2.* or E.C. 3.1. 2.14 or E.C. 3.1.1.5) activity for the production of fatty esters. In another embodiment, the host cell has OleA activity for the production of ketones. In another embodiment, the host cell has OleBCD activity for the production of internal olefins. In another embodiment, the host cell has acyl-ACP reductase (AAR) (E.C. 1.2.1.80) activity and alcohol dehydrogenase activity (E.C. 1.1.1.1.) for the production of fatty alcohols. In another embodiment, the host cell has thioesterase (E.C. 3.1.2.* or E.C. 3.1. 2.14 or E.C. 3.1.1.5) activity and decarboxylase activity for making terminal olefins. The expression of enzymatic activities in microorganisms and microbial cells is taught by U.S. Pat. Nos. 8,097,439; 8,110,093; 8,110,670; 8,183,028; 8,268,599; 8,283,143; 8,232,924; 8,372,610; and 8,530,221, which are incorporated herein by reference. In other embodiments, the host cells or microorganisms that are used to express ACP and other biosynthetic enzymes will include certain native enzyme activities that are upregulated or overexpressed in order to produce one or more particular fatty acid derivative(s) such as fatty acid derivatives. In one embodiment, the host cell has a native thioesterase (E.C. 3.1.2.* or E.C. 3.1. 2.14 or E.C. 3.1.1.5) activity for the production of fatty acids which can be increased by overexpressing the thioesterase gene.

The present disclosure includes host strains or microorganisms that express genes that code for ACP and other biosynthetic enzymes (supra). The recombinant host cells produce fatty acid derivatives and compositions and blends thereof. The fatty acid derivatives are typically recovered from the culture medium and/or are isolated from the host cells. In one embodiment, the fatty acid derivatives are recovered from the culture medium (extracellular). In another embodiment, the fatty acid derivatives are isolated from the host cells (intracellular). In another embodiment, the fatty acid derivatives are recovered from the culture medium and isolated from the host cells. The fatty acid derivatives composition produced by a host cell can be analyzed using methods known in the art, for example, GC-FID, in order to determine the distribution of particular fatty acid derivatives as well as chain lengths and degree of saturation of the components of the fatty acid derivative composition.

Examples of host cells that function as microorganisms (e.g., microbial cells), include but are not limited to cells from the genus Escherichia, Bacillus, Lactobacillus, Zymomonas, Rhodococcus, Pseudomonas, Aspergillus, Trichoderma, Neurospora, Fusarium, Humicola, Rhizomucor, Kluyveromyces, Pichia, Mucor, Myceliophtora, Penicillium, Phanerochaete, Pleurotus, Trametes, Chrysosporium, Saccharomyces, Stenotrophamonas, Schizosaccharomyces, Yarrowia, or Streptomyces. In some embodiments, the host cell is a Gram-positive bacterial cell. In other embodiments, the host cell is a Gram-negative bacterial cell. In some embodiments, the host cell is an E. coli cell. In some embodiment, the host cell is an E. coli B cell, an E. coli C cell, an E. coli K cell, or an E. coli W cell. In other embodiments, the host cell is a Bacillus lentus cell, a Bacillus brevis cell, a Bacillus stearothermophilus cell, a Bacillus lichenoformis cell, a Bacillus alkalophilus cell, a Bacillus coagulans cell, a Bacillus circulans cell, a Bacillus pumilis cell, a Bacillus thuringiensis cell, a Bacillus clausii cell, a Bacillus megaterium cell, a Bacillus subtilis cell, or a Bacillus amyloliquefaciens cell. In still other embodiments, the host cell is a Trichoderma koningii cell, a Trichoderma viride cell, a Trichoderma reesei cell, a Trichoderma longibrachiatum cell, an Aspergillus awamori cell, an Aspergillus fumigates cell, an Aspergillus foetidus cell, an Aspergillus nidulans cell, an Aspergillus niger cell, an Aspergillus oryzae cell, a Humicola insolens cell, a Humicola lanuginose cell, a Rhodococcus opacus cell, a Rhizomucor miehei cell, or a Mucor michei cell. In yet other embodiments, the host cell is a Streptomyces lividans cell or a Streptomyces murinus cell. In yet other embodiments, the host cell is an Actinomycetes cell. In some embodiments, the host cell is a Saccharomyces cerevisiae cell. In other embodiments, the host cell is a cell from a eukaryotic plant, algae, cyanobacterium, green-sulfur bacterium, green non-sulfur bacterium, purple sulfur bacterium, purple non-sulfur bacterium, extremophile, yeast, fungus, an engineered organism thereof, or a synthetic organism. In some embodiments, the host cell is light-dependent or fixes carbon. In some embodiments, the host cell has autotrophic activity. In some embodiments, the host cell has photoautotrophic activity, such as in the presence of light. In some embodiments, the host cell is heterotrophic or mixotrophic in the absence of light. In certain embodiments, the host cell is a cell from Arabidopsis thaliana, Panicum virgatum, Miscanthus giganteus, Zea mays, Botryococcuse braunii, Chlamydomonas reinhardtii, Dunaliela salina, Synechococcus Sp. PCC 7002, Synechococcus Sp. PCC 7942, Synechocystis Sp. PCC 6803, Thermosynechococcus elongates BP-1, Chlorobium tepidum, Chlorojlexus auranticus, Chromatiumm vinosum, Rhodospirillum rubrum, Rhodobacter capsulatus, Rhodopseudomonas palusris, Clostridium ljungdahlii, Clostridium thermocellum, Penicillium chrysogenum, Pichia pastoris, Saccharomyces cerevisiae, Schizosaccharomyces pombe, Pseudomonas fluorescens, or Zymomonas mobilis. In one particular embodiment, the microbial cell is from a cyanobacteria including, but not limited to, Prochlorococcus, Synechococcus, Synechocystis, Cyanothece, and Nostoc punctiforme. In another embodiment, the microbial cell is from a specific cyanobacterial species including, but not limited to, Synechococcus elongatus PCC7942, Synechocystis sp. PCC6803, and Synechococcus sp. PCC7001.

Recombinant Host Cells and Fermentation

As used herein, the term fermentation broadly refers to the conversion of organic materials into target substances by host cells, for example, the conversion of a carbon source by recombinant host cells into fatty acids or derivatives thereof by propagating a culture of the recombinant host cells in a media comprising the carbon source. The conditions permissive for the production refer to any conditions that allow a host cell to produce a desired product, such as a fatty acid or a fatty acid derivative. Similarly, the condition or conditions in which the polynucleotide sequence of a vector is expressed means any conditions that allow a host cell to synthesize a polypeptide. Suitable conditions include, for example, fermentation conditions. Fermentation conditions can include many parameters including, but not limited to, temperature ranges, levels of aeration, feed rates and media composition. Each of these conditions, individually and in combination, allows the host cell to grow. Fermentation can be aerobic, anaerobic, or variations thereof (such as micro-aerobic). Exemplary culture media include broths or gels. Generally, the medium includes a carbon source that can be metabolized by a host cell directly. In addition, enzymes can be used in the medium to facilitate the mobilization (e.g., the depolymerization of starch or cellulose to fermentable sugars) and subsequent metabolism of the carbon source.

For small scale production, the engineered host cells can be grown in batches of, for example, about 100 μL, 200 μL, 300 μL, 400 μL, 500 μL, 1 mL, 5 mL, 10 mL, 15 mL, 25 mL, 50 mL, 75 mL, 100 mL, 500 mL, 1 L, 2 L, 5 L, or 10 L; fermented; and induced to express a desired polynucleotide sequence, such as a polynucleotide sequence encoding an ACP and/or biosynthetic polypeptide. For large scale production, the engineered host cells can be grown in batches of about 10 L, 100 L, 1000 L, 10,000 L, 100,000 L, and 1,000,000 L or larger; fermented; and induced to express a desired polynucleotide sequence. Alternatively, large scale fed-batch fermentation may be carried out. The fatty acid derivative compositions described herein are found in the extracellular environment of the recombinant host cell culture and can be readily isolated from the culture medium. A fatty acid derivative may be secreted by the recombinant host cell, transported into the extracellular environment or passively transferred into the extracellular environment of the recombinant host cell culture. The fatty acid derivative is isolated from a recombinant host cell culture using routine methods known in the art.

Products Derived from Recombinant Host Cells

As used herein, the fraction of modem carbon or fM has the same meaning as defined by National Institute of Standards and Technology (NIST) Standard Reference Materials (SRMs4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), fM is approximately 1.1. Bioproducts (e.g., the fatty acid derivatives produced in accordance with the present disclosure) include biologically produced organic compounds. In particular, the fatty acid derivatives produced using the fatty acid biosynthetic pathway herein, have not been produced from renewable sources and, as such, are new compositions of matter. These new bioproducts can be distinguished from organic compounds derived from petrochemical carbon on the basis of dual carbon-isotopic fingerprinting or ¹⁴C dating. Additionally, the specific source of biosourced carbon (e.g., glucose vs. glycerol) can be determined by dual carbon-isotopic fingerprinting (see, e.g., U.S. Pat. No. 7,169,588). The ability to distinguish bioproducts from petroleum based organic compounds is beneficial in tracking these materials in commerce. For example, organic compounds or chemicals including both biologically based and petroleum based carbon isotope profiles may be distinguished from organic compounds and chemicals made only of petroleum based materials. Hence, the bioproducts herein can be followed or tracked in commerce on the basis of their unique carbon isotope profile. Bioproducts can be distinguished from petroleum based organic compounds by comparing the stable carbon isotope ratio (¹³C/¹²C) in each sample. The ¹³C/¹²C ratio in a given bioproduct is a consequence of the ¹³C/¹²C ratio in atmospheric carbon dioxide at the time the carbon dioxide is fixed. It also reflects the precise metabolic pathway. Regional variations also occur. Petroleum, C3 plants (the broadleaf), C4 plants (the grasses), and marine carbonates all show significant differences in ¹³C/¹²C and the corresponding δ¹³C values. Furthermore, lipid matter of C3 and C4 plants analyze differently than materials derived from the carbohydrate components of the same plants as a consequence of the metabolic pathway. Within the precision of measurement, ¹³C shows large variations due to isotopic fractionation effects, the most significant of which for bioproducts is the photosynthetic mechanism. The major cause of differences in the carbon isotope ratio in plants is closely associated with differences in the pathway of photosynthetic carbon metabolism in the plants, particularly the reaction occurring during the primary carboxylation (i.e., the initial fixation of atmospheric CO₂). Two large classes of vegetation are those that incorporate the C3 (or Calvin-Benson) photosynthetic cycle and those that incorporate the C4 (or Hatch-Slack) photosynthetic cycle. In C3 plants, the primary CO₂ fixation or carboxylation reaction involves the enzyme ribulose-1,5-diphosphate carboxylase, and the first stable product is a 3-carbon compound. C3 plants, such as hardwoods and conifers, are dominant in the temperate climate zones. In C4 plants, an additional carboxylation reaction involving another enzyme, phosphoenol-pyruvate carboxylase, is the primary carboxylation reaction. The first stable carbon compound is a 4-carbon acid that is subsequently decarboxylated. The CO₂ thus released is refixed by the C3 cycle. Examples of C4 plants are tropical grasses, corn, and sugar cane. Both C4 and C3 plants exhibit a range of ¹³C/¹²C isotopic ratios, but typical values are about −7 to about −13 per mil for C4 plants and about −19 to about −27 per mil for C3 plants (see, e.g., Stuiver et al. (1977) Radiocarbon 19:355). Coal and petroleum fall generally in this latter range. The ¹³C measurement scale was originally defined by a zero set by Pee Dee Belemnite (PDB) limestone, where values are given in parts per thousand deviations from this material. The δ13C values are expressed in parts per thousand (per mil), abbreviated, %, and are calculated as follows:

δ¹3C(%)=[(¹³C/¹²C)sample−(¹³C/¹²C)standard]/(¹³C/¹²C)standard×1000

Since the PDB reference material (RM) has been exhausted, a series of alternative RMs have been developed in cooperation with the IAEA, USGS, NIST, and other selected international isotope laboratories. Notations for the per mil deviations from PDB is δ¹³C. Measurements are made on CO₂ by high precision stable ratio mass spectrometry (IRMS) on molecular ions of masses 44, 45, and 46. The compositions described herein include bioproducts produced by any of the methods described herein, including, for example, fatty acid derivative products. Specifically, the bioproduct can have a δ¹³C of about −28 or greater, about −27 or greater, −20 or greater, −18 or greater, −15 or greater, −13 or greater, −10 or greater, or −8 or greater. For example, the bioproduct can have a δ¹³C of about −30 to about −15, about −27 to about −19, about −25 to about −21, about −15 to about −5, about −13 to about −7, or about −13 to about −10. In other instances, the bioproduct can have a δ¹³C of about −10, −11, −12, or −12.3. Bioproducts produced in accordance with the disclosure herein, can also be distinguished from petroleum based organic compounds by comparing the amount of ¹⁴C in each compound. Because ¹⁴C has a nuclear half-life of 5730 years, petroleum based fuels containing older carbon can be distinguished from bioproducts which contain newer carbon (see, e.g., Currie, Source Apportionment of Atmospheric Particles, Characterization of Environmental Particles, J. Buffle and H. P. van Leeuwen, Eds., 1 of Vol. I of the IUPAC Environmental Analytical Chemistry Series (Lewis Publishers, Inc.) 3-74, (1992)). The basic assumption in radiocarbon dating is that the constancy of ¹⁴C concentration in the atmosphere leads to the constancy of ¹⁴C in living organisms. However, because of atmospheric nuclear testing since 1950 and the burning of fossil fuel since 1850, ¹⁴C has acquired a second, geochemical time characteristic. Its concentration in atmospheric CO₂, and hence in the living biosphere, approximately doubled at the peak of nuclear testing, in the mid-1960s. It has since been gradually returning to the steady-state cosmogenic (atmospheric) baseline isotope rate (¹⁴C/¹²C) of about 1.2×10⁻¹², with an approximate relaxation “half-life” of 7-10 years. This latter half-life must not be taken literally; rather, one must use the detailed atmospheric nuclear input/decay function to trace the variation of atmospheric and biospheric¹⁴C since the onset of the nuclear age. It is this latter biospheric¹⁴C time characteristic that holds out the promise of annual dating of recent biospheric carbon. ¹⁴C can be measured by accelerator mass spectrometry (AMS), with results given in units of fraction of modern carbon (fM). fM is defined by National Institute of Standards and Technology (NIST) Standard Reference Materials (SRMs) 4990B and 4990C. As used herein, fraction of modern carbon or fM has the same meaning as defined by National Institute of Standards and Technology (NIST) Standard Reference Materials (SRMs) 4990B and 4990C, known as oxalic acids standards HOxI and HOxII, respectively. The fundamental definition relates to 0.95 times the ¹⁴C/¹²C isotope ratio HOxI (referenced to AD 1950). This is roughly equivalent to decay-corrected pre-Industrial Revolution wood. For the current living biosphere (plant material), fM is approximately 1.1. The compositions described herein include bioproducts that can have an fM¹⁴C of at least about 1. For example, the bioproduct of the disclosure can have an fM¹⁴C of at least about 1.01, an fM¹⁴C of about 1 to about 1.5, an fM¹⁴C of about 1.04 to about 1.18, or an fM¹⁴C of about 1.111 to about 1.124.

Another measurement of ¹⁴C is known as the percent of modern carbon (pMC). For an archaeologist or geologist using ¹⁴C dates, AD 1950 equals zero years old. This also represents 100 pMC. Bomb carbon in the atmosphere reached almost twice the normal level in 1963 at the peak of thermo-nuclear weapons. Its distribution within the atmosphere has been approximated since its appearance, showing values that are greater than 100 pMC for plants and animals living since AD 1950. It has gradually decreased over time with today's value being near 107.5 pMC. This means that a fresh biomass material, such as corn, would give a ¹⁴C signature near 107.5 pMC. Petroleum based compounds will have a pMC value of zero. Combining fossil carbon with present day carbon will result in a dilution of the present day pMC content. By presuming 107.5 pMC represents the ¹⁴C content of present day biomass materials and 0 pMC represents the ¹⁴C content of petroleum based products, the measured pMC value for that material will reflect the proportions of the two component types. For example, a material derived 100% from present day soybeans would give a radiocarbon signature near 107.5 pMC. If that material was diluted 50% with petroleum based products, it would give a radiocarbon signature of approximately 54 pMC. A biologically based carbon content is derived by assigning 100% equal to 107.5 pMC and 0% equal to 0 pMC. For example, a sample measuring 99 pMC will give an equivalent biologically based carbon content of 93%. This value is referred to as the mean biologically based carbon result and assumes all the components within the analyzed material originated either from present day biological material or petroleum based material. A bioproduct comprising one or more fatty acid derivatives as described herein can have a pMC of at least about 50, 60, 70, 75, 80, 85, 90, 95, 96, 97, 98, 99, or 100. In other instances, a fatty acid derivative described herein can have a pMC of between about 50 and about 100; about 60 and about 100; about 70 and about 100; about 80 and about 100; about 85 and about 100; about 87 and about 98; or about 90 and about 95. In yet other instances, a fatty acid derivative described herein can have a pMC of about 90, 91, 92, 93, 94, or 94.2.

Screening Fatty Acid Derivative Compositions Produced by Recombinant Host Cells

To determine if conditions are sufficient to allow expression, a host cell can be cultured, for example, for about 4, 8, 12, 24, 36, or 48 hours. During and/or after culturing, samples can be obtained and analyzed to determine if the conditions allow expression. For example, the host cells in the sample or the medium in which the host cells were grown can be tested for the presence of a desired product. When testing for the presence of a product, assays, such as, but not limited to, TLC, HPLC, GC/FID, GC/MS, LC/MS, MS, can be used. Recombinant host cell cultures are screened at the 96 well plate level, 1 liter, 5 liter tank level and at a 1000 L pilot plant scale using a GC/FID assay for total Fatty Acid Species (FAS).

Effect of an Increase in ACP on Fatty Alcohol Production

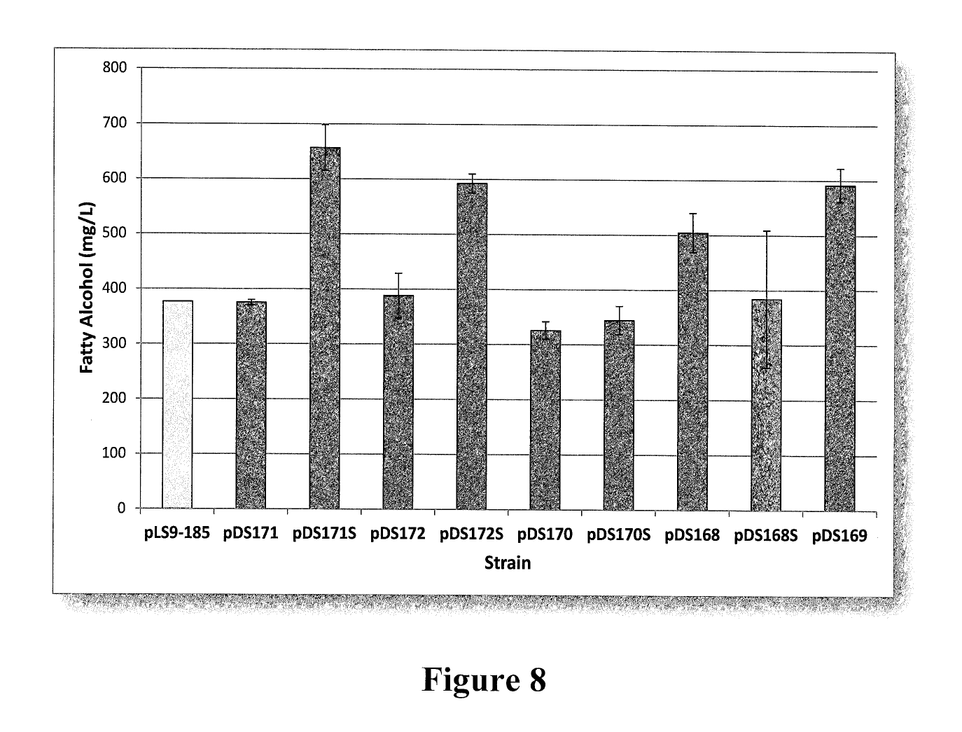

Recombinant host cells can be engineered to overexpress ACP (e.g., cyanobacterial ACPs, see Table 3, infra). In some embodiments, recombinant host cell may be further engineered to include a polynucleotide sequence encoding one or more fatty acid biosynthetic polypeptides, for example, a polypeptide having thioesterase (TE) activity and a polypeptide having carboxylic acid reductase (CAR) activity, wherein the recombinant host cell synthesizes fatty aldehydes and/or fatty alcohols. In other embodiments, the recombinant host cell is further engineered to comprise a polynucleotide sequence encoding TE activity, CAR activity and alcohol dehydrogenase activity wherein the recombinant host cell synthesizes fatty alcohols. In still other embodiments, a recombinant host cell is engineered to include a polynucleotide sequence encoding a polypeptide having acyl-ACP reductase (AAR) activity wherein the recombinant host cell synthesizes fatty aldehydes and fatty alcohols; or to include a polynucleotide sequence encoding a polypeptide having AAR activity and alcohol dehydrogenase activity wherein the recombinant host cell synthesizes fatty alcohols. In some cases the recombinant host cell is engineered to include a polynucleotide sequence encoding a polypeptide having fatty alcohol forming acyl-CoA reductase (FAR) activity wherein the recombinant host cell synthesizes fatty alcohols. Overexpression of the nucleic acid sequences encoding cyanobacterial ACPs (see Table 3, infra) was shown to improve fatty alcohol titer and yield (see Example 1 and FIG. 8, infra).

Effect of an Increase in ACP on Fatty Ester Production

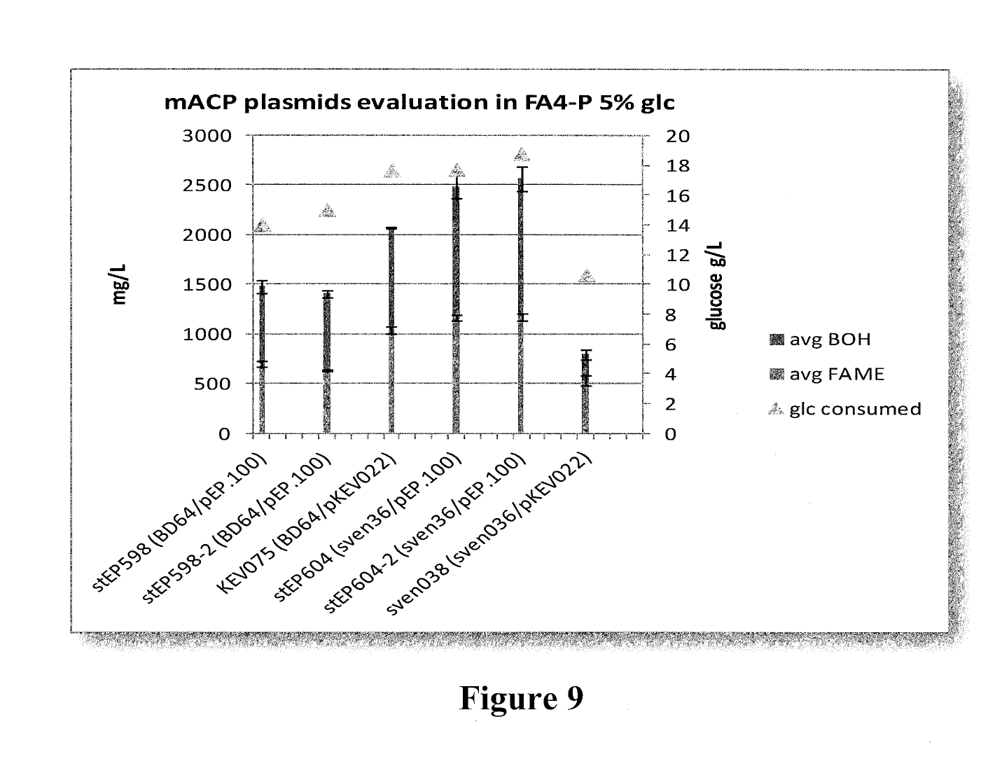

Recombinant host cells can be engineered to overexpress ACP (e.g., M. aquaeolei VT8 ACP (SEQ ID NO: 122, NCBI: YP_{—}959135.1). In some embodiments, recombinant host cell may be further engineered to include a polynucleotide sequence encoding one or more fatty acid biosynthetic polypeptides, for example, a polypeptide having ester synthase (ES) activity; or one or more polypeptides having thioesterase (TE) activity, acyl-CoA synthase/synthetase (fadD) activity and ester synthase activity, wherein the recombinant host cell synthesizes fatty esters (e.g., FAME, FAEE). In some embodiments, a recombinant host cell may be engineered to include a polynucleotide sequence encoding a polypeptide having ester synthase activity wherein the recombinant host cell synthesizes fatty esters (one enzyme system, see FIG. 5); or a polynucleotide sequence encoding a polypeptide having thioesterase activity, acyl-CoA synthase activity and ester synthase activity wherein the recombinant host cell synthesizes fatty esters (three enzyme system, see FIG. 5). Overexpression of the nucleic acid sequence encoding M. aquaeolei VT8 ACP (SEQ ID NO: 122, NCBI: YP_{—}959135.1) was shown to improve fatty acyl methyl ester (FAME) titer and yield (see Examples 2 and 3 and FIGS. 9-15, infra).

Effect of an Increase in ACP on Hydrocarbon Production

Recombinant host cells can be engineered to overexpress ACP (e.g., cyanobacterial ACPs, see Table 3, infra). In some embodiments, recombinant host cell may be further engineered to include a polynucleotide sequence encoding one or more fatty acid biosynthetic polypeptides, for example, a polypeptide having acyl-ACP reductase (AAR) activity and a polypeptide having decarbonylase (ADC) activity, wherein the recombinant host cell synthesizes alkanes. Overexpression of the nucleic acid sequences encoding cyanobacterial ACPs (see Table 3, infra) was shown to improve alkane titer and yield (see Example 4, infra).

In some embodiments, the alkane is a C₃-C₂₅ alkane. For example, the alkane is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅ or C₂₆ alkane. In some embodiments, the alkane is tridecane, methyltridecane, nonadecane, methylnonadecane, heptadecane, methylheptadecane, pentadecane, or methylpentadecane. The alkane may be a straight chain alkane, a branched chain alkane, or a cyclic alkane. In certain embodiments, the method further includes culturing the host cell in the presence of a saturated fatty acid derivative, and the hydrocarbon produced is an alkane or an alkene. In certain embodiments, the saturated fatty acid derivative is a C₆-C₂₆ fatty acid derivative substrate. In particular embodiments, the fatty acid derivative substrate is 2-methylicosanal, icosanal, octadecanal, tetradecanal, 2-methyloctadecanal, stearaldehyde, or palmitaldehyde. In some embodiments, the method further includes isolating the alkane from the host cell or from the culture medium. In other embodiments, the method further includes cracking or refining the alkane.

In other embodiments, the hydrocarbon produced is an alkene. In some embodiments, the alkene is a C₃-C₂₅ alkene. For example, the alkene is a C₃, C₄, C₅, C₆, C₇, C₈, C₉, C₁₀, C₁₁, C₁₂, C₁₃, C₁₄, C₁₅, C₁₆, C₁₇, C₁₈, C₁₉, C₂₀, C₂₁, C₂₂, C₂₃, C₂₄, C₂₅ or C₂₆ alkene. In some embodiments, the alkene is pentadecene, heptadecene, methylpentadecene, or methylheptadecene. The alkene may be a straight chain alkene, a branched chain alkene, or a cyclic alkene. In some embodiments, a recombinant host cell is engineered to include a polynucleotide sequence encoding a polypeptide having acyl-CoA reductase (AAR) activity and aldehyde decarbonylase (ADC) activity, wherein the recombinant host cell synthesizes hydrocarbons (alkanes and alkenes). In other embodiments, the recombinant host cell is engineered to include a polynucleotide sequence encoding a polypeptide having thioesterase activity, carboxylic acid reductase activity and aldehyde decarbonylase activity, wherein the recombinant host cell synthesizes hydrocarbons (alkanes and alkenes). In still other embodiments, the recombinant host cell is engineered to include a polynucleotide sequence encoding a polypeptide having acyl-CoA reductase activity and OleA activity, wherein the recombinant host cell synthesizes aliphatic ketones; a polynucleotide sequence encoding a polypeptide having OleABCD activity, wherein the recombinant host cell synthesizes internal olefins; or a polynucleotide sequence encoding a polypeptide having thioesterase activity and decarboxylase activity, wherein the recombinant host cell synthesizes terminal olefins.

Fatty Acid Derivative Compositions and their Use

A fatty acid is a carboxylic acid with a long aliphatic tail (chain), which is either saturated or unsaturated. Most naturally occurring fatty acids have a chain of an even number of carbon atoms, from 4 to 28. Fatty acids are usually derived from triglycerides. When they are not attached to other molecules, they are known as free fatty acids. Fatty acids are usually produced industrially by the hydrolysis of triglycerides, with the removal of glycerol. Palm, soybean, rapeseed, coconut oil and sunflower oil are currently the most common sources of fatty acids. The majority of fatty acids derived from such sources are used in human food products. Coconut oil and palm kernel oil (are made of mainly of 12 and 14 carbon fatty acids). These are particularly suitable for further processing to surfactants for washing and cleansing agents as well as cosmetics. Palm, soybean, rapeseed, and sunflower oil, as well as animal fats such as tallow, contain mainly long-chain fatty acids (e.g., C18, saturated and unsaturated) which are used as raw materials for polymer applications and lubricants. Ecological and toxicological studies suggest that fatty acid-derived products based on renewable resources have more favorable properties than petrochemical-based substances.

Fatty aldehydes are used to produce many specialty chemicals. For example, aldehydes are used to produce polymers, resins (e.g., BAKELITE resin), dyes, flavorings, plasticizers, perfumes, pharmaceuticals, and other chemicals, some of which may be used as solvents, preservatives, or disinfectants. In addition, certain natural and synthetic compounds, such as vitamins and hormones, are aldehydes, and many sugars contain aldehyde groups. Fatty aldehydes can be converted to fatty alcohols by chemical or enzymatic reduction.

Fatty alcohols have many commercial uses. Worldwide annual sales of fatty alcohols and their derivatives are in excess of U.S. $1 billion. The shorter chain fatty alcohols are used in the cosmetic and food industries as emulsifiers, emollients, and thickeners. Due to their amphiphilic nature, fatty alcohols behave as nonionic surfactants, which are useful in personal care and household products, such as, for example, detergents. In addition, fatty alcohols are used in waxes, gums, resins, pharmaceutical salves and lotions, lubricating oil additives, textile antistatic and finishing agents, plasticizers, cosmetics, industrial solvents, and solvents for fats. The disclosure also provides a surfactant composition or a detergent composition comprising a fatty alcohol produced by any of the methods described herein. One of ordinary skill in the art will appreciate that, depending upon the intended purpose of the surfactant- or detergent composition, different fatty alcohols can be produced and used. For example, when the fatty alcohols described herein are used as a feedstock for surfactant or detergent production, one of ordinary skill in the art will appreciate that the characteristics of the fatty alcohol feedstock will affect the characteristics of the surfactant or detergent composition produced. Hence, the characteristics of the surfactant or detergent composition can be selected for by producing particular fatty alcohols for use as a feedstock. A fatty alcohol-based surfactant and/or detergent composition described herein can be mixed with other surfactants and/or detergents well known in the art. In some embodiments, the mixture can include at least about 10%, at least about 15%, at least about 20%, at least about 30%, at least about 40%, at least about 50%, at least about 60%, or a range bounded by any two of the foregoing values, by weight of the fatty alcohol. In other examples, a surfactant or detergent composition can be made that includes at least about 5%, at least about 10%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of a fatty alcohol that includes a carbon chain that is 8, 9, 10, 11, 12, 13, 14, 15, 16, 17, 18, 19, 20, 21, or 22 carbons in length. Such surfactant or detergent compositions can also include at least one additive, such as a microemulsion or a surfactant or detergent from non-microbial sources such as plant oils or petroleum, which can be present in the amount of at least about 5%, at least about 10%, at least about 15%, at least about 20%, at least about 30%, at least about 40%, at least about 50%, at least about 60%, at least about 70%, at least about 80%, at least about 85%, at least about 90%, at least about 95%, or a range bounded by any two of the foregoing values, by weight of the fatty alcohol.

Esters have many commercial uses. For example, biodiesel, an alternative fuel, is made of esters (e.g., fatty acid methyl esters, fatty acid ethyl esters, etc.). Some low molecular weight esters are volatile with a pleasant odor, which makes them useful as fragrances or flavoring agents. In addition, esters are used as solvents for lacquers, paints, and varnishes. Furthermore, some naturally occurring substances, such as waxes, fats, and oils are made of esters. Esters are also used as softening agents in resins and plasticizers, flame retardants, and additives in gasoline and oil. In addition, esters can be used in the manufacture of polymers, films, textiles, dyes, and pharmaceuticals.

Hydrocarbons have many commercial uses. For example, shorter chain alkanes are used as fuels. Longer chain alkanes (e.g., from five to sixteen carbons) are used as transportation fuels (e.g., gasoline, diesel, or aviation fuel). Alkanes having more than sixteen carbon atoms are important components of fuel oils and lubricating oils. Even longer alkanes, which are solid at room temperature, can be used, for example, as a paraffin wax. In addition, longer chain alkanes can be cracked to produce commercially valuable shorter chain hydrocarbons Like short chain alkanes, short chain alkenes are used in transportation fuels. Longer chain alkenes are used in plastics, lubricants, and synthetic lubricants. In addition, alkenes are used as a feedstock to produce alcohols, esters, plasticizers, surfactants, tertiary amines, enhanced oil recovery agents, fatty acids, thiols, alkenylsuccinic anhydrides, epoxides, chlorinated alkanes, chlorinated alkenes, waxes, fuel additives, and drag flow reducers.

Ketones are used commercially as solvents. For example, acetone is frequently used as a solvent, but it is also a raw material for making polymers. Ketones are also used in lacquers, paints, explosives, perfumes, and textile processing. In addition, ketones are used to produce alcohols, alkenes, alkanes, imines, and enamines.

Lubricants are typically composed of olefins, particularly polyolefins and alpha-olefins. Lubricants can either be refined from crude petroleum or manufactured using raw materials refined from crude petroleum. Obtaining these specialty chemicals from crude petroleum requires a significant financial investment as well as a great deal of energy. It is also an inefficient process because frequently the long chain hydrocarbons in crude petroleum are cracked to produce smaller monomers. These monomers are then used as the raw material to manufacture the more complex specialty chemicals.

### EXAMPLES

The following specific examples are intended to illustrate the disclosure and should not be construed as limiting the scope of the claims.

From an LB culture growing in a 96 well plate, 30 μL of LB culture was used to inoculate 270 μL FA2P media (see Table 2, infra), which was then incubated for approximately 16 hours at 32° C. on a shaker to generate an overnight seed. 30 μL of the overnight seed was used to inoculate 300 μL FA4P media+2% MeOH+1 mM isopropyl β-D-1-thiogalactopyranoside (IPTG) (see Table 2, infra). The cultures were incubated at 32° C. on a shaker for 24 hours, after which they were extracted using the standard extraction protocol detailed below.

TABLE 2
Media Names And Formulations

| Media Name | Formulation | | |
|---|---|---|---|
| FA2P Media | 1 | X | P-lim 5x Salt Soln |
| | 2 | g/L | 100 g/L NH4Cl |
| | 1 | mg/ml | 10 mg/mL Thiamine |
| | 1 | mM | 1M MgSO4 |
| | 0.1 | mM | 1M CaCl2 |
| | 30 | g/L | 500 g/L glucose |
| | 1 | X | 1000x TM2 |
| | 10 | mg/L | 10 g/L Fe Citrate |
| | 100 | mM | 2M BisTris (pH 7.0) |
| FA4P Media | 0.5 | X | P-lim 5x Salt Soln |
| | 2 | g/L | 100 g/L NH4Cl |
| | 1 | mg/ml | 10 mg/mL Thiamine |
| | 1 | mM | 1M MgSO4 |
| | 0.1 | mM | 1M CaCl2 |
| | 50 | g/L | 500 g/L glucose |
| | 1 | X | 1000x TM2 |
| | 10 | mg/L | 10 g/L Fe Citrate |
| | 100 | mM | 2M BisTris (pH 7.0) |

Fatty Acid Species Standard Extraction Protocol:

To each well to be extracted 40 μL of 1M HCl, then 300 μL butyl acetate with 500 mg/L C11-FAME was added as internal standard was added. The 96 well plate was heat-sealed using a plate sealer (ALPS-300; Abgene, ThermoScientific, Rockford, Ill.), and shaken for 15 minutes at 2000 rpm using MixMate (Eppendorf, Hamburg, Germany). After shaking, the plate was centrifuged for 10 minutes at 4500 rpm at room temperature (Allegra X-15R, rotor SX4750A, Beckman Coulter, Brea, Calif.) to separate the aqueous and organic layers. 50 μL of the organic layer was transferred to a 96 well plate (96-well plate, polypropylene, Corning, Amsterdam, The Netherlands). The plate was heat sealed then stored at −20° C. until it was evaluated by GC-FID using the Upstream_Biodiesel_FAME_{—} BOH-FAME-underivitized. method described below (infra).

Upstream Biodiesel FAME BOH FAME Underivitized Method:

1 mL of sample was injected onto a UFM column (cat #: UFMC00001010401, Thermo Fisher Scientific, Waltham, Mass.) in a Trace GC Ultra (Thermo Fisher Scientific, Waltham, Mass.) with a flame ionization detector (FID). The instrument was set up to detect C8 to C18 FAME and C8 to C18 β-OH FAME.

The protocols detailed above represent standard conditions, which may be modified to change the extraction volume or another parameter, as necessary to optimize the analytical results.

### Example 1

### Increased Acyl Carrier Protein (ACP)—Mediated Flux Through the Fatty Acid Synthesis Pathway

The acp genes from several cyanobacteria were cloned downstream from the Synechococcus elongatus PCC7942 acyl-ACP reductase (AAR) in plasmid pLS9-185, which is a pCL1920 derivative (3-5 copies/cell). The sfp gene (Accession No. X63158; SEQ ID NO: 11) from Bacillus subtilis encodes a phosphopantetheinyltransferase which is involved in conversion of the inactive apo-ACP protein to the active holo-ACP protein. This phosphopantetheinyltransferase (SEQ ID NO: 12) with broad substrate specificity was cloned downstream of the respective acp genes. The plasmids listed in Table 3 (infra) were constructed to carry out a number of studies.

TABLE 3
Plasmids Coexpressing Cyanobacterial ACP with and without B. Subtilis
sfp Downstream from S. elongatus PCC7942 AAR
(in base plasmid pLS9-185)

| ACP Source | Without sfp | With sfp |
|---|---|---|
| Synechococcus elongatus 794 | pDS168 | pDS168S |
| Synechocystis sp. 6803 | pDS169 | not available |
| Prochlorococcus marinus MED4 | pDS170 | pDS170S |
| Nostocpunctiforme 73102 | pDS171 | pDS171S |
| Nostoc sp. 7120 | pDS172 | pDS172S |

Fatty Acid Production

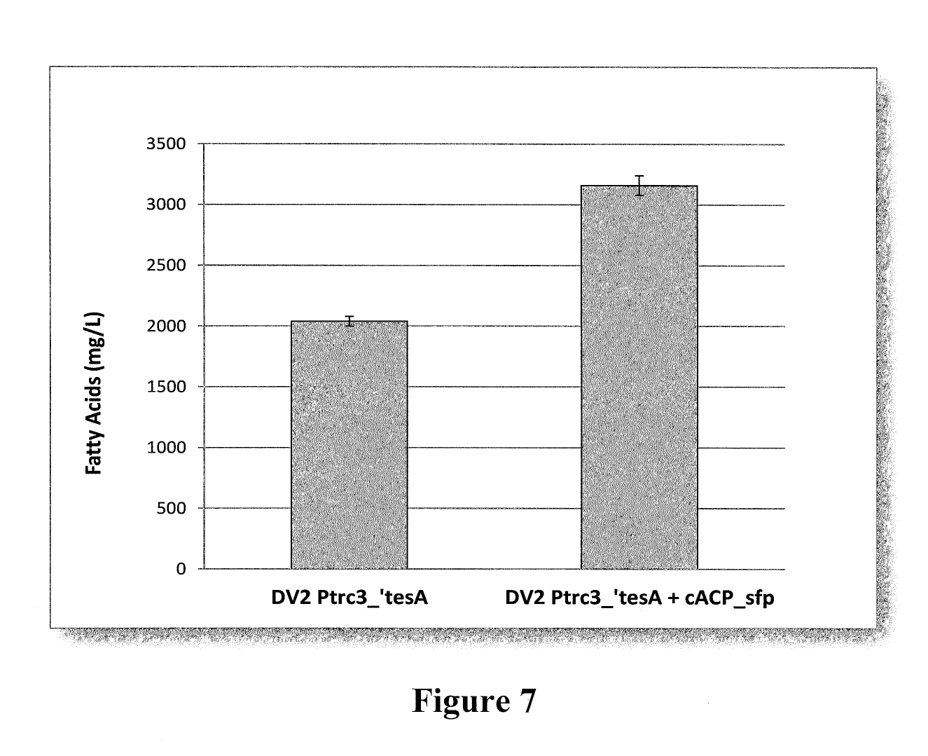

In order to evaluate if the overexpression of an ACP can increase free fatty acid production, one cyanobacterial ACP gene with sfp was amplified from pDS171s (see Table 3, supra) and cloned downstream from ‘tesA (leaderless thioesterase gene) into a pCL vector. The resulting operon was put under the control of the Ptrc3 promoter, which provides slightly lower transcription levels than the Ptrc wildtype promoter. The construct was cloned into E. coli DV2 and evaluated for fatty acid production. The control strain contained the identical plasmid but without cyanobacterial ACP and B. subtilis sfp. The results from a standard microtiter plate fermentation experiment are shown in FIG. 7. As shown, a significant improvement in fatty acid titer was observed in the host strain coexpressing the heterologous ACP demonstrating that ACP overexpression can be beneficial for fatty acid production, in this case presumably by increasing the flux through the fatty acid biosynthetic pathway.

Fatty Alcohol Production

Several cyanobacterial acp genes were cloned downstream of the Nostoc 73102 acyl-ACP reductase (AAR; SEQ ID NO: 80) in pLS9-185. This plasmid is pCL1920-based and is present at about 3-5 copies/cell. In addition, in some plasmids, the sfp gene from Bacillus subtilis, encoding phosphopantetheinyl transferase, was cloned downstream of the respective acp genes. All the acp genes were cloned with a synthetic RBS into the EcoRI site immediately downstream of the aar gene in pLS9-185 using IN-FUSION technology (IN-FUSION HD cloning kit; Clonetech Laboratories, Inc.). The EcoRI site was reconstructed downstream of the acp gene. Similarly, the B. subtilis sfp gene was IN-FUSION cloned into this EcoRI site along with a synthetic RBS.

Synechocystis 7942 acp (SEQ ID NO: 7) was amplified from plasmid pEPO9 with primers 1681FF (SEQ ID NO: 13) and 1681FR (SEQ ID NO: 14). This PCR product was cloned using the IN-FUSION kit (supra) into the EcoRI site of plasmid pLS9-185 to form plasmid pDS168.

Synechocystis 6803 acp (SEQ ID NO: 3) was amplified from plasmid pTB044 using primers 1691FF (SEQ ID NO: 15) and 1691FR (SEQ ID NO: 16). This PCR product was cloned using the IN-FUSION kit (supra) into the EcoRI site of plasmid pLS9-185 to form plasmid pDS169.

Prochlorococcus marinus MED4 acp (SEQ ID NO: 5) was amplified from plasmid pEP07 using primers 1701FF (SEQ ID NO: 17) and 170IFR (SEQ ID NO: 18). This PCR product was cloned using the IN-FUSION kit (supra) into the EcoRI site of plasmid pLS9-185 to form plasmid pDS170.

Nostoc 73102 acp (SEQ ID NO: 1) was amplified from plasmid pEP11 using primers 1711FF (SEQ ID NO: 19) and 171IFR (SEQ ID NO: 20). This PCR product was cloned using the IN-FUSION kit (supra) into the EcoRI site of plasmid pLS9-185 to form plasmid pDS171.

Nostoc 7120 acp (SEQ ID NO: 9) was amplified from plasmid pTB045 using primers 1721FF (SEQ ID NO: 21) and 1721FR (SEQ ID NO: 22). This PCR product was cloned using the IN-FUSION kit (supra) into the EcoRI site of plasmid pLS9-185 to form plasmid pDS172.

The synthetic sfp gene (encoding a modified 4′-phosphopantetheinyltransferase) was amplified and cloned into the EcoRI site of plasmids pDS168-pDS172. The sfp+synthetic RBS was amplified with one of the following forward primers: 168SIFF (SEQ ID NO: 23) 170S1FF, (SEQ ID NO: 24) 171SIFF (SEQ ID NO: 25). The same reverse primer was used for each amplification, as follows: 168SIFR (SEQ ID NO: 26). The 168S PCR product was cloned into EcoRI-restricted pDS168 using IN-FUSION technology (supra) to form pDS168S. The 170S PCR product was cloned into EcoRI-restricted pDS170 using IN-FUSION technology (supra) to form pDS170S. The 171S PCR product was cloned into EcoRI-restricted pDS171 using IN-FUSION technology (supra) to form pDS171S. The 172S PCR product was cloned into EcoRI-restricted pDS172 using IN-FUSION technology (supra) to form pDS172S.

The results from standard shake flask fermentation experiments are shown in FIG. 8. As shown, significant improvement in fatty alcohol titers were observed in host strains containing the plasmids pDS168 and pDS169 (see Table 3, supra), demonstrating that ACP overexpression can be beneficial to fatty alcohol production, in this case presumably by aiding in the recognition, affinity and/or turnover of acyl-ACPs by the heterologous terminal pathway enzyme. In addition, significant improvement in titer was observed in host strains containing the plasmids pDS171S and pDS172S. These plasmids contain the Nostoc7120 or 73102_acp genes followed by the sfp gene. Host strains containing pDS169 (Synechocystis 6803_acp) also exhibited improvement in titer. This was shown to be reproducible in several independent experiments. Native alcohol dehydrogenases converted aldehyde to alcohols in vivo.

### Example 2

### Increased Acyl Carrier Protein (ACP)-Mediated Flux Through the Fatty Acid Synthesis Pathway—Fatty Ester Production

Herein, methyl ester production was shown to be improved by overexpression of the M. aquaeolei VT8 acyl carrier protein (mACP). The protein sequence of ACP from Marinobacter aquaeolei VT8 (SEQ ID NO: 122) is identical to the protein sequence of ACP from Marinobacter hydrocarbonoclasticus (DSM8798; ATCC49840; SEQ ID NO: 124). However, the nucleic acid sequence for M. aquaeolei VT8 (SEQ ID NO: 121) differs from the nucleic acid sequence for DSM8798 (SEQ ID NO: 123) by one base pair (i.e., silent mutation).

Host cell strains (i.e., sven.036, based on MG1655 with DfadE, DtonA, rph+ and ilvG+T5_ifab138 T5_fadR) previously engineered to produce fatty esters (i.e., FAME) were further modified to carry a production plasmid, designated pKEV022 (carrying genes for ester synthase, ACC from Corynebacterium glutamicum, and birA from Corynebacterium glutamicum), in which mACP was cloned behind the birA gene (i.e., pEP.100 which is the same as pKEV022-mACP). Here, birA was used to enhance ACC activity, as it ligates biotin to AccB (biotin carboxyl carrier protein).

These strains produced higher fatty acid methyl ester (FAME) yields and titers in plate fermentation and in 5 L bioreactor fermentation as compared to fatty ester host cell production strains which do not contain mACP. M. aquaeolei VT8 acyl carrier protein (mACP) which is also referred to as Marinobacter ACP was amplified from plasmid pNH153L using primers EP343 (SEQ ID NO: 27) and EP345 (SEQ ID NO: 28) and then cloned via the IN-FUSION kit (supra) into pKEV022 plasmid behind the birA gene. pNH153L was generated by amplifying mACP from a genomic DNA preparation of the M. aquaeolei VT8.

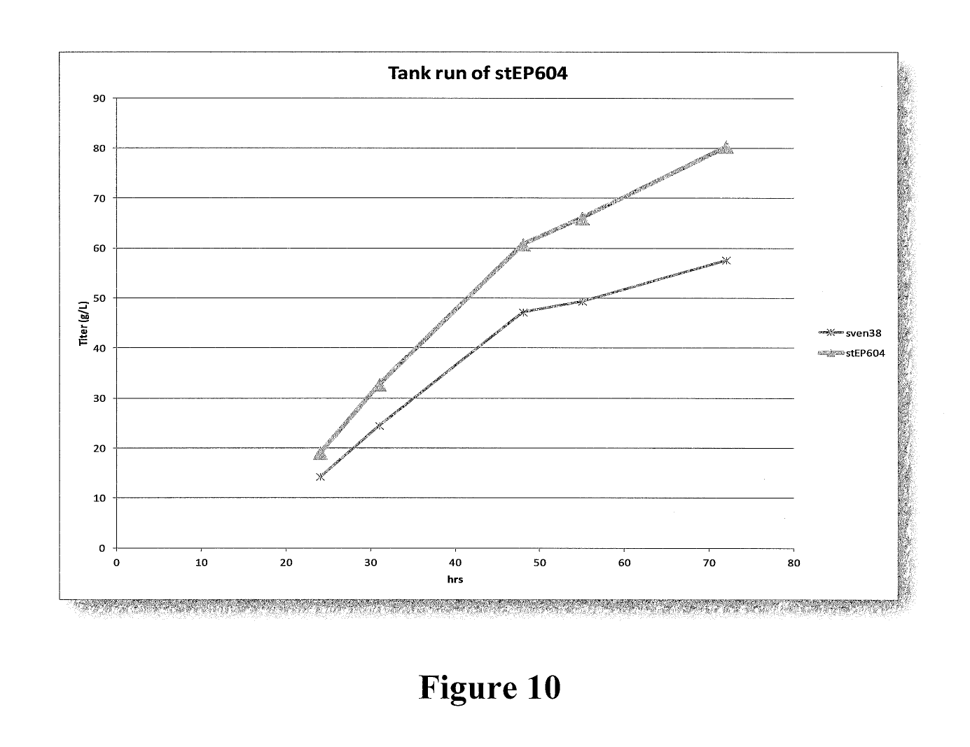

An optimized IGR sequence [birA-TAAtagaggaggataactaaATG-mACP (SEQ ID NO: 29)] was used in front of the mACP. The pKEV022 plasmid backbone for the infusion cloning was amplified with primers EP342 (SEQ ID NO: 30) and EP344 (SEQ ID NO: 31). The sequences of the ester synthase, the ACC-birA and the mACP genes in the pEP.100 plasmid were sequence verified. Plasmid pEP.100 was transformed into BD64 and sven036 E. coli strains. The resulting strains were named stEP598 and stEP604, respectively. The Sven036 strain is isogenic to BD64 with the additional feature of rph+ and ilvG+ corrections and a T5 promoter in front of the ifab138 operon (see PCT/US13/35037). Two colonies from each strain along with the appropriate controls (KEV075=BD64/pKEV022 and sven038=sven036/pKEV022) were tested in triplicates using the Protocol Ester Screening in Plates described above. FIG. 9 shows the results of plate fermentation of strains containing THE pEP.100 plasmid. As depicted, the stEP604 strains show a surprisingly high titer improvement (3 fold) over the control sven038 strain. The same plasmid in the BD64 strain background results in slightly lower titers than the control KEV075 strain. Based on these fermentation results, stEP604 was evaluated next in 5 L bioreactors. FIG. 10 illustrates the tank data for stEP604. As shown, StEP604 had consistently higher titer over the control (sven38) throughout the run.

These results show that cloning M. aquaeolei VT8 ACP behind birA in the pKEV022 plasmid and expressing it in the sven036 background resulted in a 10% yield improvement and greater than a 35% increase in titer when compared to the control sven038 strain. These results suggest that overexpression of ACP, including ACPs from other microorganisms, can effectively increase the yield of fatty acid derivatives. The expression level of M. aquaeolei VT8 ACP may be further optimized through RBS or promoter libraries resulting in even greater yield improvements and greater increases in titer.

### Example 3

### Overexpression of Escherichia coli or Marinobacter aquaeolei VT8 ACP Increases Flux Through the Fatty Acid Synthesis Pathway—Fatty Ester Production

FAME produced by recombinant host cells can be used in the production of commercial biodiesel, however; optimization of fermentation processes on an economically viable commercial scale requires maximizing the titer and yield of FAME production. Candidate commercial strains can be identified in high throughput screens, as well as by culture in 5 L bioreactors. In this study, overexpression of E. coli ACP or M. aquaeolei VT8 ACP, respectively, was shown to increase the fatty acyl methyl ester (FAME) titer and yield from recombinant host cells. It has been shown above that host cell strains genetically modified to express M. aquaeolei VT8 ACP (mACP), for example, plasmid pKEV022, produce higher titers of FAME (see Example 2, supra). In this example, E. coli ACP was evaluated under similar conditions. E. coli ACP (ecACP) and M. aquaeolei VT8s ACP (mACP) were tested in combination with different ester synthase variants to see if they were compatible with enzyme variants.

Plasmid Construction

Plasmid pSven.036 includes pKEV022-ecACP; and plasmid pSven.037 includes pSHU18-ecACP. The ecACP was amplified from production host strain sven.036 using primers oSV44 (SEQ ID NO: 32) and oSV45 (SEQ ID NO: 33). The gene sequence was then cloned into pKEV022 and pSHU18 plasmid behind the birA gene via IN-FUSION kit (supra) cloning. An optimized IGR sequence (underlined in primer oSV44) was used in front of the Escherichia coli ACP. The pKEV022/pSHU18 plasmid backbone for the infusion cloning was amplified with primers EP342 (SEQ ID NO: 30) and EP344 (SEQ ID NO: 31). The cloning reaction was first transformed in STELLAR chemically competent cells and then sequence was verified before purification of the new plasmids pSven.036 and pSven.037. A similar strategy was also used to clone mACP into different ester synthase variants where the plasmid pEP100 was used as a template to amplify the mACP using primers EP343 (SEQ ID NO: 27) and EP345 (SEQ ID NO: 28). The resulting plasmids are shown in Table 4 below (infra). D+ refers to the presence of the accDA, accBC and birA genes downstream of the ester synthase (the accDA, accBC and birA genes came from Corynebacterium glutamicum).

TABLE 4
Description of Plasmids

| Plasmid | Description |
|---|---|
| pSven.025 | pSHU18_macp |
| pSven.034 | pKEV018_mACP |
| pSven.035 | pSven.023_mACP |
| pSven.038 | pKASH010_D+_mACP |
| pSven.039 | pKASH011_D+_mACP |
| pSven.040 | pKEV028_mACP |
| pSven.041 | pKASH5_D+_mACP |

Fermentation Results

All plasmids shown in Table 4 were transformed into the production host, GLPH-077. The strains GLPH-077 and GLPH-009 were derived from sven.036 by selecting for resistance to phage. Sven.036 contains corrections for frame-shift mutation of ilvG and rph naturally present in WT MG1655 strains and also has a T5 promoter driving the ifab138 operon (supra) which facilitates overexpression of the genes involved in fatty acid biosynthesis. Four individual transformants were picked and compared against appropriate controls using the Protocol Ester Screening in plates described above. The titer and β-OH content of the FAME produced by production host GLPH-077 transformed with plasmids shown in Table 4 was compared to the titer and β-OH content of the FAME produced by production host GLPH-077 expressing the same ester synthase variants without overexpression of ACP. The strains used in this study are listed in Table 5, containing ester synthase variants from Marinobacter hydrocarbonoclasticus.

TABLE 5
Strain Descriptions

| Strain Moniker | Description |
|---|---|
| sven.312 | GLPH-077 pSven.034 |
| sven.313 | GLPH-077 pSven.035 |
| sven.314 | GLPH-077 pSven.036 |
| sven.315 | GLPH-077 pSven.037 |
| sven.316 | GLPH-077 pSven.038 |
| sven.317 | GLPH-077 pSven.039 |
| sven.318 | GLPH-077 pSven.040 |
| sven.320 | GLPH-077 pKEV018 |
| sven.321 | GLPH-077 pSven.023 |
| sven.322 | GLPH-077 pSHU018 |
| sven.323 | GLPH-077 pKASH010_D+ |
| sven.324 | GLPH-077 pKASH011_D+ |
| sven.325 | GLPH-077 pKEV028 |
| sven.205 | GLPH-077 pKEV022 |
| sven.209 | GLPH-077 pSHU018 |
| stEP.604 | sven.036 pEP100 |
| sven.340 | GLPH-077 pEP100 |
| shu129 | sven36 pSHU18 |
| sven241 | GLPH-009 pSven.025 |
| sven.227 | GLPH-009 pSven.023 |

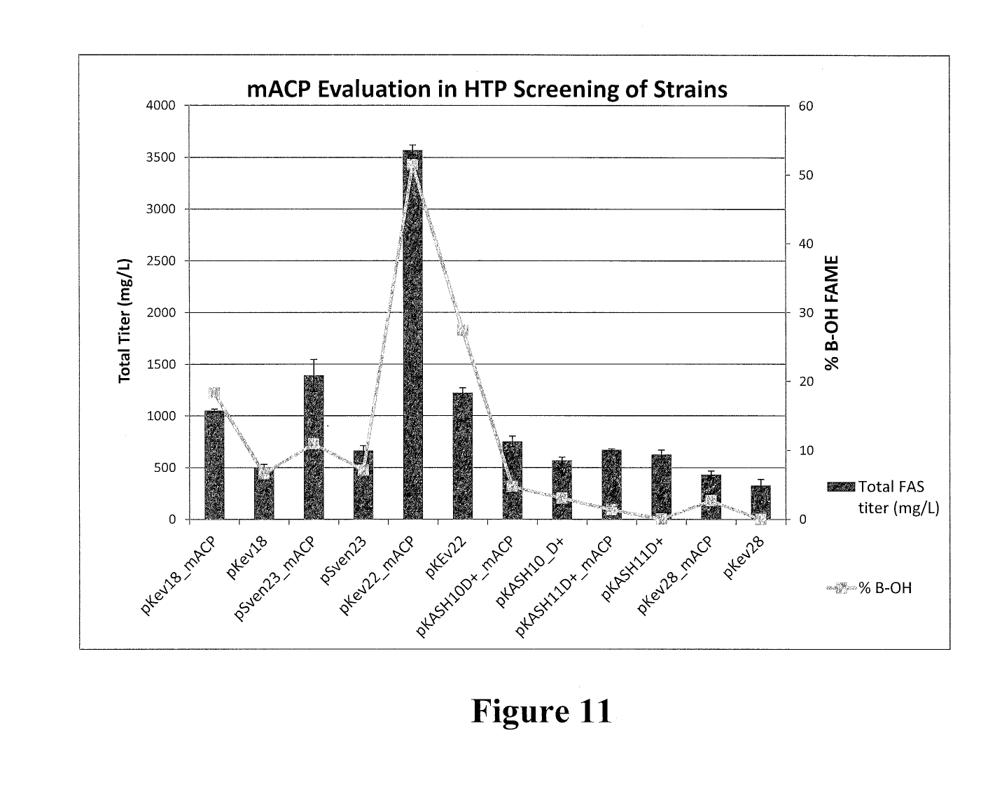

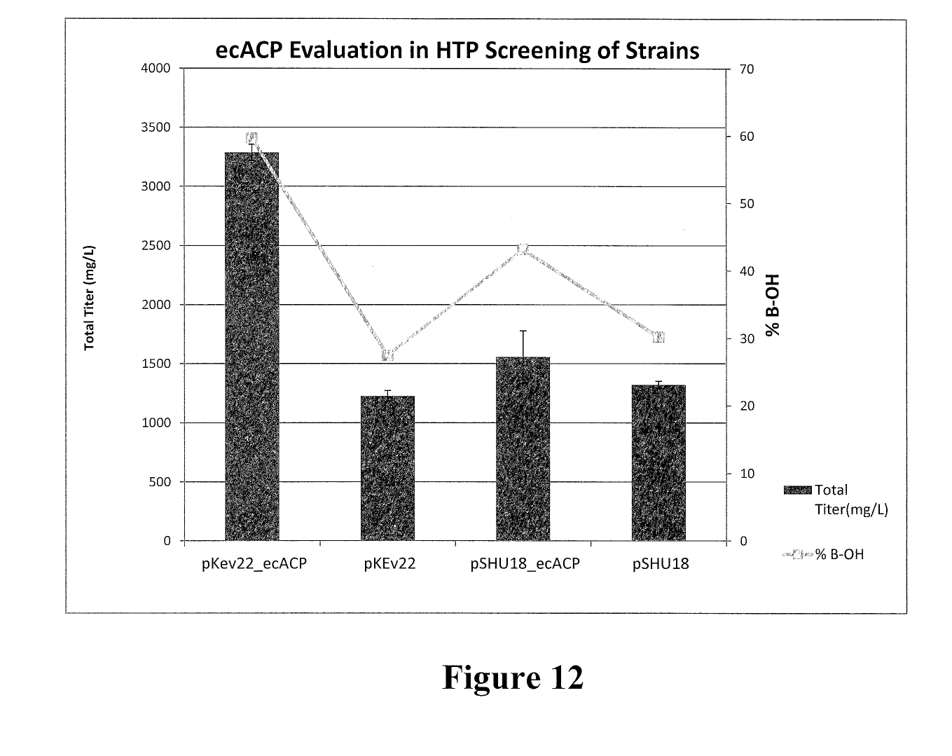

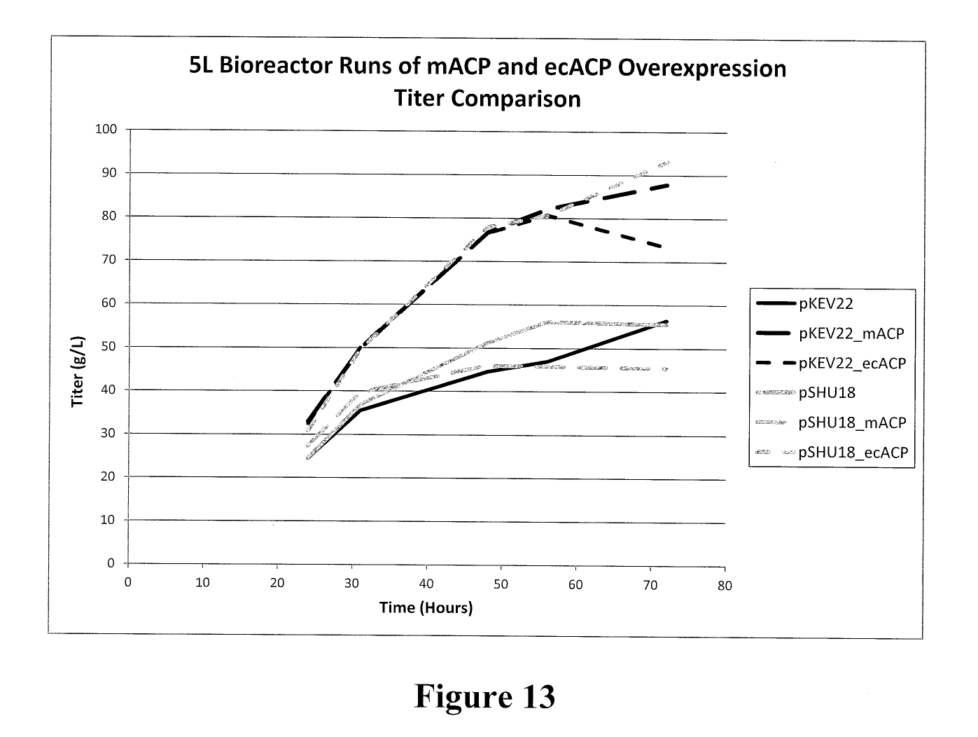

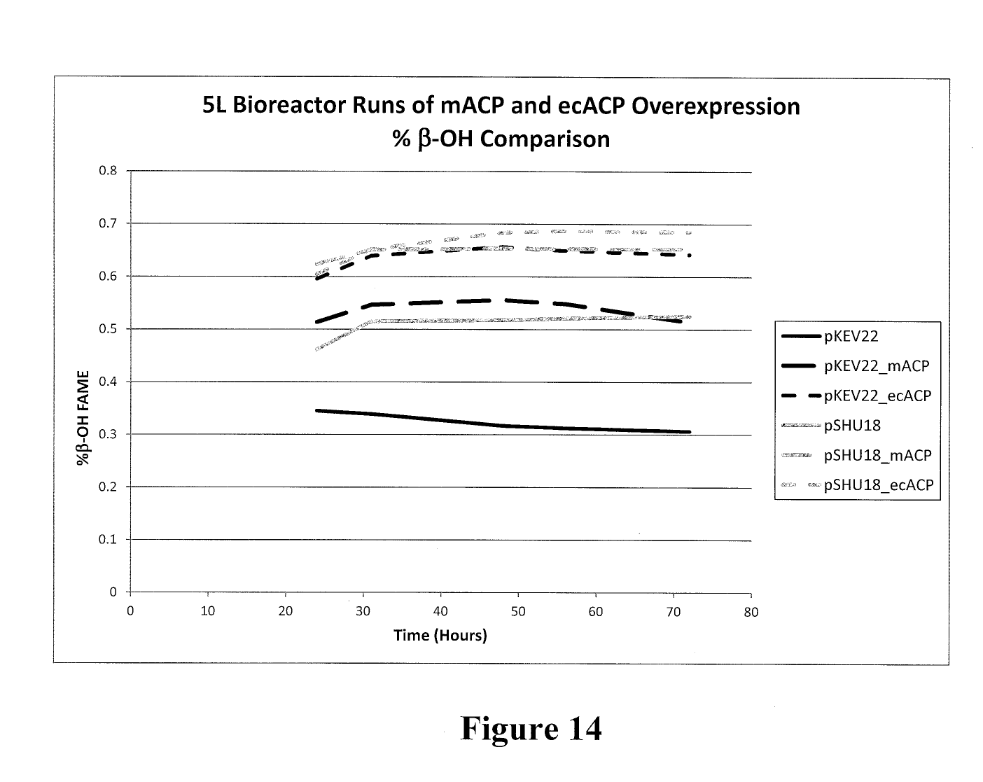

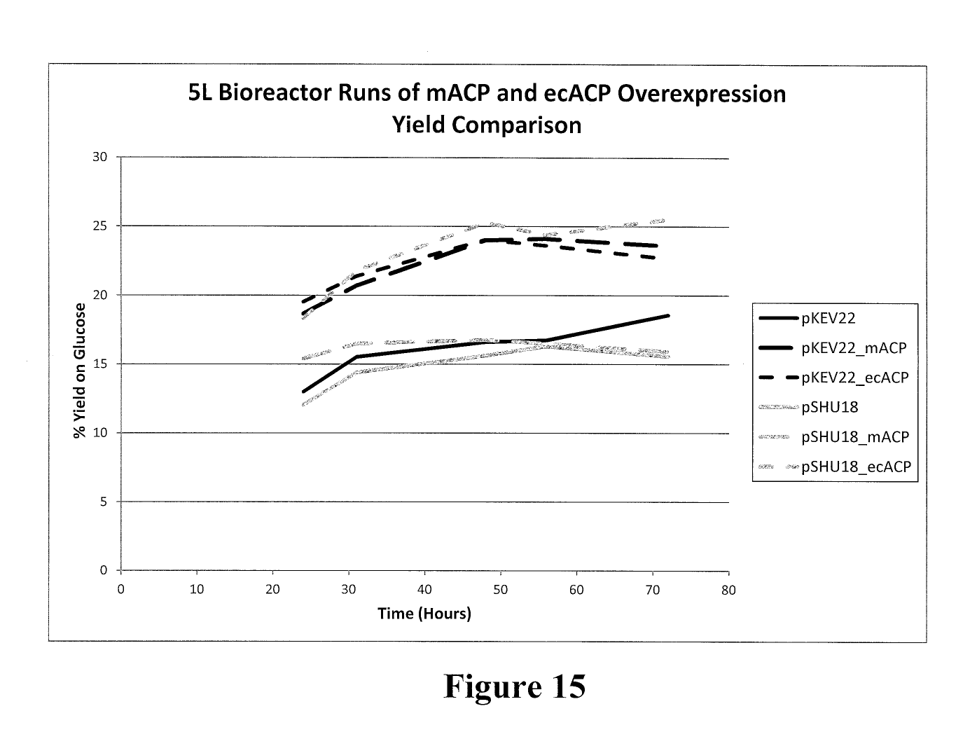

As can be seen in FIG. 11, the strains with mACP overexpression showed a significant increase of total FAME titer over the respective controls, in particular, when using the pKEV022 plasmid (pSven.037 includes pKEV022-mACP), which produced a titer that was approximately 3-fold that of the control strain (sven.315 and sven.205). FIG. 12 illustrates the overexpression of ecACP in pKEV022 and pSHU018. Based on this fermentation results, the strains were run in 5 L bioreactors. FIG. 13 shows the bioreactor titer data of mACP and ecACP overexpression. pSHU18 with the ecACP was shown to out-perform other ester synthase variants in terms of total Fatty Acid Species (FAS) produced. FIG. 14 illustrates β-OH FAME production in bioreactors. pSHU18 with overexpression of ecACP produced approximately 68% β-OH FAME. FIG. 15 illustrates bioreactor data comparing yield on glucose. pSHU18 with overexpression of ecACP clearly exhibited a higher yield than the other strains tested in this study. This data shows that cloning ecACP behind birA in pSHU18 plasmid and expressing it in GLPH77 background (sven.315), resulted in an 8% improvement in yield and a 6% improvement in FAS titer compared to step604. Also sven.315 had a two-fold improvement in titer and 60% in terms of yield relative to sven.241 (which has mACP overexpressed in the pSHU18 plasmid). Strain sven.315 exhibits a 64% greater yield and a 67% increase in titer for FAS. When sven.313 (which has mACP overexpressed in the pSven.023 plasmid) was compared to sven.227, there was an observed improvement of 36% in titer and 32% in yield. This data indicates that the presence of ecACP or mACP results in a large increase in yield and titer of FAS.

The sequences of mACP and ecACP were compared using the NCBI tool “BLASTp”. The results were as follows: Query 1 sequence (77 amino acids in length).

Method: Compositional Matrix Adjust
Identities = 62/76 (82%), Positives = 68/76
(89%), Gaps = 0/76 (0%)
Query1 MSTIEERVKKIIGEQLGVKQEEVTNNASFVEDLGADSLDTVEL
VMALEEEFDTEIPDEEA 60
MST + EERVKKI + EQLGVK + EV N +
SFVEDLGADSLDTVELVMALEEEF + TEIPDEEA
Subject 1 MSTVEERVKKIVCEQLGVKESEVQNTSSFVEDLGADSLDT
VELVMALEEEFLTEIPDEEA 60
Query 61 EKITTVQAAIDYINGH 76
EK + TVQ AIDYI H
Subject61 EKLGTVQDAIDYIVAH76

The sequence alignment results indicate that the mACP and ecACP proteins are 82% identical and 89% similar to each other in terms of amino acid residues. This suggests that ACPs from other organisms (that have a certain sequence similarity) may have a similar effect (as exemplified mACP and ecACP sequences) in enhancing production of fatty acid derivatives such as fatty alcohols and fatty esters. The expression level of ACP in the cell can be further optimized through IGR libraries. Further improvements in yield may be obtained by integration of the ACP gene in the E. coli chromosome and/or by expression under the control of a medium to strong promoter. Promoter libraries may be built using these strains. Alternatively, ACPs from other organisms may be tested.

### Example 4

### Overexpression of Escherichia coli or Marinobacter hydrocarbonoclasticus ACP Increases Flux Through the Fatty Acid Synthesis Pathway—Alkane Production

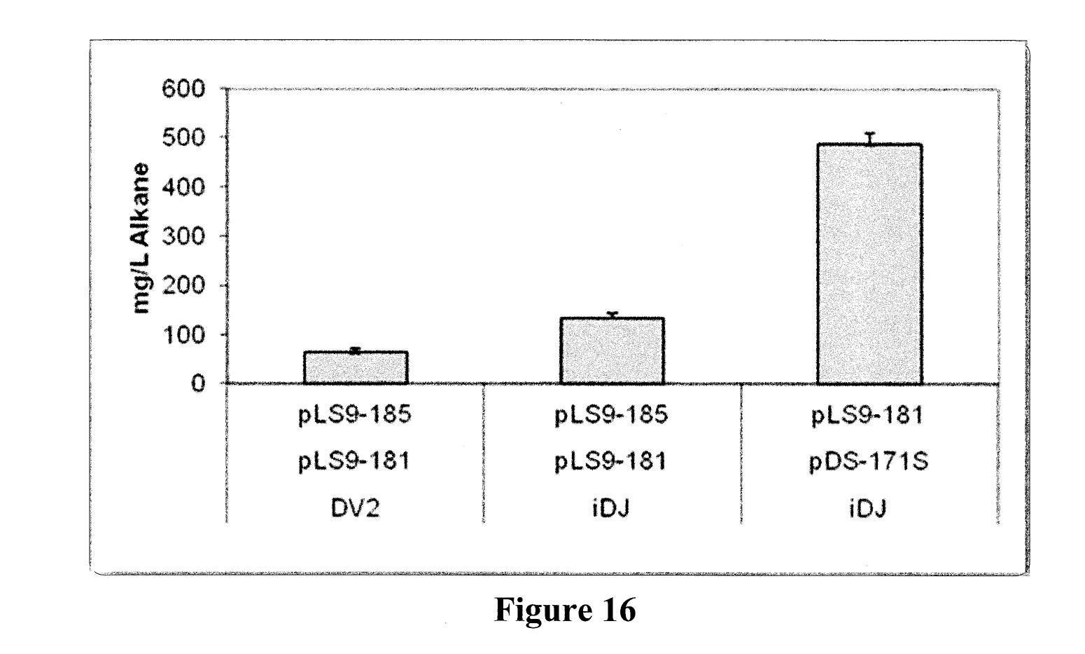

A number of cyanobacterial acp genes were cloned downstream from the Nostoc 73102 acyl-ACP reductase (SEQ ID NO: 80) present in pLS9-185. Plasmid pDS171S contains Nostoc 73102 acp cloned with a synthetic RBS into the EcoRI site immediately downstream of the aar gene in pLS9-185. The sfp gene from Bacillus subtilis, was cloned downstream of the respective acp genes. These plasmids were co-expressed with plasmid pLS9-181, which contains the ADC (aldehyde decarbonylase from Nostoc PCC73102; SEQ ID NO: 38). The strain containing both plasmids was subjected to a standard fermentation protocol at 32° C. with the addition of 25 mM Mn²⁺. FIG. 16 shows the average amount of alkane that was produced 24 hours post-induction (triplicates+/−standard error). A significant 5 fold improvement (see column 3 in FIG. 16) in alkane titer was observed in the strain containing the plasmid pDS171S. The control (no acp/sfp) was pLS9-185. The results indicate that expression of Nostoc 73102 acp+sfp improved alkane production.

The cyanobacterial acp+sfp genes can be supplied in several forms, e.g., integration at a site in the chromosome either associated with the alkane operon or present as a separate unit. The expression of acp and sfp may be varied by manipulation of the promoter and/or ribosome binding site. The results suggest that expression of active cyanobacterial ACP may facilitate an increased titer/yield of alkanes by recombinant host production strains.

The results of Examples 1 through 4 illustrate the creation of new recombinant host cell strains with enhanced and altered abilities to convert raw materials such as glucose into fatty acids, fatty esters, fatty alcohols, and fatty alkanes. Thus, it has been shown herein that the overexpression of ACPs improves the production of fatty acid derivatives via recombinant host cells and results in higher titer, higher yield and higher productivity when compared to corresponding wild type cells. All sequence identifying numbers (SEQ ID NOS) are listed in Table 6 below (see Sequence Listing for complete sequences information).

TABLE 6
Table of Sequences

| SEQ ID NO.: | Type | Name |
|---|---|---|
| 1 | nucleic acid seq. | Nostoc punctiforme PCC 73102_acp Accession# YP_001867863 |
| 2 | amino acid seq. | Nostoc punctiforme PCC 73102_acp Accession# YP_001867863 |
| 3 | nucleic acid seq. | Synechocystis sp. PCC 6803_acp Accession # NP_440632.1 |
| 4 | amino acid seq. | Synechocystis sp. PCC 6803_acp Accession # NP_440632.1 |
| 5 | nucleic acid seq. | Prochlorococcus marinus subsp. pastoris str. CCMP1986_acp Accession# NP_893725.1 |
| 6 | amino acid seq. | Prochlorococcus marinus subsp. pastoris str. CCMP1986_acp Accession# NP_893725.1 |
| 7 | nucleic acid seq. | Synechococcus elongatus PCC 7942_acp Accession# YP_399555 |
| 8 | amino acid seq. | Synechococcus elongatus PCC 7942_acp Accession# YP_399555 |
| 9 | nucleic acid seq. | Nostoc sp. PCC 7120_acp Accession# NP_487382.1 |
| 10 | amino acid seq. | Nostoc sp. PCC 7120_acp Accession# NP_487382.1 |
| 11 | nucleic acid seq. | B. subtilis sfp (synthesized) as in accession# X63158.1 |
| 12 | amino acid seq. | B. subtilis sfp (synthesized) as in accession# X63158.1 |
| 13 | primer seq. | 168IFF |
| 14 | primer seq. | 168IFR |
| 15 | primer seq. | 169IFF |
| 16 | primer seq. | 169IFR |
| 17 | primer seq. | 170IFF |
| 18 | primer seq. | 170IFR |
| 19 | primer seq. | 171IFF |
| 20 | primer seq. | 171IFR |
| 21 | primer seq. | 172IFF |
| 22 | primer seq. | 172IFR |
| 23 | primer seq. | 168SIFF |
| 24 | primer seq. | 170S1FF |
| 25 | primer seq. | 171SIFF |
| 26 | primer seq. | 168SIFR |
| 27 | primer seq. | EP343 |
| 28 | primer seq. | EP345 |
| 29 | primer seq. | optimized IGR seq. in front of Marinobacter ACP |
| 30 | primer seq. | EP342 |
| 31 | primer seq. | EP344 |
| 32 | primer seq. | oSV044 |
| 33 | primer seq. | oSV045 |
| 34 | nucleic acid seq. | Synechococcus elongatus PCC7942 YP.sub.--400610 (Synpcc7942.sub.--1593) aldehyde |
| | | decarbonylase |
| 35 | amino acid seq. | Synechococcus elongatus PCC7942 YP.sub.--400610 (Synpcc7942.sub.--1593) aldehyde |
| | | decarbonylase |
| 36 | nucleic acid seq. | Synechocystis sp. PCC6803 sll0208 (NP_442147) aldehyde decarbonylase |
| 37 | amino acid seq. | Synechocystis sp. PCC6803 sll0208 (NP_442147) aldehyde decarbonylase |
| 38 | nucleic acid seq. | Nostoc punctiforme PCC73102 Npun02004178 (ZP_00108838) aldehyde decarbonylase |
| 39 | amino acid seq. | Nostoc punctiforme PCC73102 Npun02004178 (ZP_00108838) aldehyde decarbonylase |
| 40 | nucleic acid seq. | Nostoc sp. PCC7120 alr5283 (NP.sub.--489323) aldehyde decarbonylase |
| 41 | amino acid seq. | Nostoc sp. PCC7120 alr5283 (NP.sub.--489323) aldehyde decarbonylase |
| 42 | nucleic acid seq. | Acaryochloris marina MBIC11017 AM1_4041 aldehyde decarbonylase |
| 43 | amino acid seq. | Acaryochloris marina MBIC11017 AM1_4041 aldehyde decarbonylase |
| 44 | nucleic acid seq. | Thermosynechococcus elongatus BP-1 tll1313 aldehyde decarbonylase |
| 45 | amino acid seq. | Thermosynechococcus elongatus BP-1 tll1313 aldehyde decarbonylase |
| 46 | nucleic acid seq. | Synechococcus sp. JA-3-3A CYA_0415 aldehyde decarbonylase |
| 47 | amino acid seq. | Synechococcus sp. JA-3-3A CYA_0415 aldehyde decarbonylase |
| 48 | nucleic acid seq. | Gloeobacter violaceus PCC7421 gll3146 aldehyde decarbonylase |
| 49 | amino acid seq. | Gloeobacter violaceus PCC7421 gll3146 aldehyde decarbonylase |
| 50 | nucleic acid seq. | Prochlorococcus marinus MIT9313 PMT1231 (NP_895059) aldehyde decarbonylase |
| 51 | amino acid seq. | Prochlorococcus marinus MIT9313 PMT1231 (NP_895059) aldehyde decarbonylase |
| 52 | nucleic acid seq. | Prochlorococcus mariunus CCMP1986 PMM0532 aldehyde decarbonylase |
| 53 | amino acid seq. | Prochlorococcus mariunus CCMP1986 PMM0532 aldehyde decarbonylase |
| 54 | nucleic acid seq. | Prochlorococcus marinus str. NATL2A PMN2A_1863 aldehyde decarbonylase |
| 55 | amino acid seq. | Prochlorococcus marinus str. NATL2A PMN2A_1863 aldehyde decarbonylase |
| 56 | nucleic acid seq. | Synechococcus sp. RS9917_09941 aldehyde decarbonylase |
| 57 | amino acid seq. | Synechococcus sp. RS9917_09941 aldehyde decarbonylase |
| 58 | nucleic acid seq. | Synechococcus sp. RS9917_12945 aldehyde decarbonylase |
| 59 | amino acid seq. | Synechococcus sp. RS9917_12945 aldehyde decarbonylase |
| 60 | nucleic acid seq. | Cyanothece sp. ATCC51142 cce_0778 (YP_001802195) aldehyde decarbonylase |
| 61 | amino acid seq. | Cyanothece sp. ATCC51142 cce_0778 (YP_001802195) aldehyde decarbonylase |
| 62 | nucleic acid seq. | Cyanothece sp. PCC7425 Cyan7425_0398 (YP_002481151) aldehyde decarbonylase |
| 63 | amino acid seq. | Cyanothece sp. PCC7425 Cyan7425_0398 (YP_002481151) aldehyde decarbonylase |
| 64 | nucleic acid seq. | Cyanothece sp. PCC7425 Cyan7425_2986 (YP_002483683) aldehyde decarbonylase |
| 65 | amino acid seq. | Cyanothece sp. PCC7425 Cyan7425_2986 (YP_002483683) aldehyde decarbonylase |
| 66 | nucleic acid seq. | Anabaena variabilis ATCC29413 YP_323043 (Ava_2533) aldehyde decarbonylase |
| 67 | amino acid seq. | Anabaena variabilis ATCC29413 YP_323043 (Ava_2533) aldehyde decarbonylase |
| 68 | nucleic acid seq. | Synechococcus elongatus PCC6301 YP_170760 aldehyde decarbonylase |
| 69 | amino acid seq. | Synechococcus elongatus PCC6301 YP_170760 aldehyde decarbonylase |
| 70 | nucleic acid seq. | Synechococcus elongatus PCC7942 YP_400611 (Synpcc7942_1594) Acyl-CoA |
| | | Reductase |
| 71 | amino acid seq. | Synechococcus elongatus PCC7942 YP_400611 (Synpcc7942_1594) Acyl-CoA |
| | | Reductase (AAR) |
| 72 | nucleic acid seq. | Synechocystis sp. PCC6803 sll0209 (NP_442146) AAR |
| 73 | amino acid seq. | Synechocystis sp. PCC6803 sll0209 (NP_442146) AAR |
| 74 | nucleic acid seq. | Cyanothece sp. ATCC51142 cce_1430 (YP_001802846) AAR |
| 75 | amino acid seq. | Cyanothece sp. ATCC51142 cce_1430 (YP_001802846) AAR |
| 76 | nucleic acid seq. | Prochlorococcus marinus CCMP1986 PMM0533 (NP_892651) AAR |
| 77 | amino acid seq. | Prochlorococcus marinus CCMP1986 PMM0533 (NP_892651) AAR |
| 78 | nucleic acid seq. | Gloeobacter violaceus PCC7421 NP_96091 (gll3145) AAR |
| 79 | amino acid seq. | Gloeobacter violaceus PCC7421 NP_96091 (gll3145) AAR |
| 80 | nucleic acid seq. | Nostoc punctiforme PCC73102 ZP_00108837 (Npun02004176) AAR |
| 81 | amino acid seq. | Nostoc punctiforme PCC73102 ZP_00108837 (Npun02004176) AAR |
| 82 | nucleic acid seq. | Anabaena variabilis ATCC29413 YP_323044 (Ava_2534) AAR |
| 83 | amino acid seq. | Anabaena variabilis ATCC29413 YP_323044 (Ava_2534) AAR |
| 84 | nucleic acid seq. | Synechococcus elongatus PCC6301 YP_170761 (syc0051_d) AAR |
| 85 | amino acid seq. | Synechococcus elongatus PCC6301 YP_170761 (syc0051_d) AAR |
| 86 | nucleic acid seq. | Nostoc sp. PCC7120 alr5284 (NP_489324) AAR |
| 87 | amino acid seq. | Nostoc sp. PCC7120 alr5284 (NP_489324) AAR |
| 88 | nucleic acid seq. | Mycobacterium smegmatis (YP_889972.1; CarB) |
| 89 | nucleic acid seq. | Mycobacterium smegmatis (CarB60) |
| 90 | amino acid seq. | Mycobacterium smegmatis (YP_889972.1; CarB) |
| 91 | nucleic acid seq. | CarA; ABK75684 |
| 92 | amino acid seq. | CarA; ABK75684 |
| 93 | nucleic acid seq. | wild type ester synthase, ES9/DSM8798 from Marinobacter hydrocarbonoclasticus, |
| | | GenBank Accession No. ABO21021) |
| 94 | amino acid seq. | wild type ester synthase, ES9/DSM8798 from Marinobacter hydrocarbonoclasticus, |
| | | GenBank Accession No. ABO21021) |
| 95 | nucleic acid seq. | 9B12 variant of SEQ ID NO: 94 |
| 96 | amino acid seq. | 9B12 variant - D7N, A179V, V381F |
| 97 | nucleic acid seq. | 9B12* variant of SEQ ID NO: 93 |
| 98 | amino acid seq. | 9B12* variant - D7N, A179V, Q348R, V381F |
| 99 | nucleic acid seq. | pKEV018 (KEV040) ester synthase |
| 100 | amino acid seq. | pKEV018 (KEV040) ester synthase |
| 101 | nucleic acid seq. | pKEV022 (KEV075) ester synthase |
| 102 | amino acid seq. | pKEV022 (KEV075) ester synthase |
| 103 | nucleic acid seq. | pKEV028 (KEV085) ester synthase |
| 104 | amino acid seq. | pKEV028 (KEV085) ester synthase |
| 105 | nucleic acid seq. | pSHU10 (variant of SEQ ID NO: 1) T5S, S15G, P111S, V171R, P188R, F317W, S353T, |
| | | V409L, S442G |
| 106 | amino acid seq. | pSHU10 (variant of SEQ ID NO: 1) T5S, S15G, P111S, V171R, P188R, F317W, S353T, |
| | | V409L, S442G |
| 107 | nucleic acid seq. | KASH8 (variant of SEQ ID NO: 33) T5S, S15G, K78F, P111S, V171R, P188R, S192V, |
| | | A243R, F317W, K349H, S353T, V409L, S442G |
| 108 | amino acid seq. | KASH8 (variant of SEQ ID NO: 18; SHU10) T5S, S15G, K78F, P111S, V171R, P188R, |
| | | S192V, A243R, F317W, K349H, S353T, V409L, S442G |
| 109 | nucleic acid seq. | KASH32 (variant of SEQ ID NO: 18; SHU10) T5S, S15G, V76L, P111S, V171R, P188R, |
| | | K258R, S316G, F317W, S353T, M360R, V409L, S442G |
| 110 | amino acid seq. | KASH32 (variant of SEQ ID NO: 18; SHU10) T5S, S15G, V76L, P111S, V171R, P188R, |
| | | K258R, S316G, F317W, S353T, M360R, V409L, S442G |
| 111 | nucleic acid seq. | KASH40 (variant of SEQ ID NO: 18; SHU10) T5S, S15G, P111S, V171R, P188R, |
| | | Q244G, S267G, G310V, F317W, A320C, S353T, Y366W, V409L, S442G |
| 112 | amino acid seq. | KASH40 (variant of SEQ ID NO: 18; SHU10) T5S, S15G, P111S, V171R, P188R, |
| | | Q244G, S267G, G310V, F317W, A320C, S353T, Y366W, V409L, S442G |
| 113 | nucleic acid seq. | KASH60 (variant of SEQ ID NO: 18; SHU10) S15G, P111S, V155G, P166S, V171R, |
| | | P188R, F317W, Q348A, S353T, V381F, V409L, S442G |
| 114 | amino acid seq. | KASH60 (variant of SEQ ID NO: 18; SHU10) S15G, P111S, V155G, P166S, V171R, |
| | | P188R, F317W, Q348A, S353T, V381F, V409L, S442G |
| 115 | nucleic acid seq. | KASH61 (variant of SEQ ID NO: 18; SHU10) S15G, L39S, D77A, P111S, V171R, |
| | | P188R, T313S, F317W, Q348A, S353T, V381F, V409L, I420V, S442G |
| 116 | amino acid seq. | KASH61 (variant of SEQ ID NO: 18; SHU10) S15G, L39S, D77A, P111S, V171R, |
| | | P188R, T313S, F317W, Q348A, S353T, V381F, V409L, I420V, S442G |
| 117 | nucleic acid seq. | KASH78 (variant of SEQ ID NO: 18; SHU10) T5S, S15G, T24W, T44F, P111S, I146L, |
| | | V171R, P188R, D307N, F317W, S353T, V409L, S442G |
| 118 | amino acid seq. | KASH78 (variant of SEQ ID NO: 18; SHU10) T5S, S15G, T24W, T44F, P111S, I146L, |
| | | V171R, P188R, D307N, F317W, S353T, V409L, S442G |
| 119 | amino acid seq. | ABO21020: 376 seq. |
| 120 | nucleic acid seq. | ABO21020: 376 seq. |
| 121 | nucleic acid seq. | Marinobacter aquaeolei VT8 ACP (YP_959135.1) |
| 122 | amino acid seq. | Marinobacter aquaeolei VT8 ACP (YP_959135.1) |
| 123 | nucleic acid seq. | Marinobacter hydrocarbonoclasticus acp (YP_005429338.1) |
| 124 | amino acid seq. | Marinobacter hydrocarbonoclasticus acp (YP_005429338.1) |

As is apparent to one with skill in the art, various modifications and variations of the above aspects and embodiments can be made without departing from the spirit and scope of this disclosure. Such modifications and variations are within the scope of this disclosure.

What is claimed is:

1. A recombinant host cell, comprising:
(a) a polynucleotide sequence encoding an exogenous acyl carrier protein (ACP); and
(b) a polynucleotide sequence encoding an exogenous fatty acid derivative biosynthetic protein, wherein the recombinant host cell produces a fatty acid derivative composition.

2. The recombinant host cell of claim 1, wherein said recombinant host cell produces said fatty acid derivative composition with a higher titer, a higher yield or a higher productivity when cultured in medium containing a carbon source under conditions effective to overexpress said polynucleotide sequence of (a) and (b), as compared to a corresponding wild type host cell propagated under the same conditions as the recombinant host cell.

3. The recombinant host cell of claim 1, wherein the fatty acid derivative composition comprises a fatty acid derivative selected from the group consisting of a fatty acid, a fatty alcohol, a fatty ester, a fatty aldehyde, an alkane, an alkene, an olefin, and a ketone.

4. The recombinant host cell of claim 1, wherein the fatty acid derivative biosynthetic protein has thioesterase activity and the fatty acid derivative composition comprises a fatty acid.

5. The recombinant host cell of claim 4, further comprising a protein that has carboxylic acid reductase (CAR) activity, wherein the fatty acid derivative composition comprises a fatty alcohol.

6. The recombinant host cell of claim 1, wherein the fatty acid derivative biosynthetic protein has acyl ACP reductase (AAR) activity and the fatty acid derivative composition comprises a fatty alcohol.

7. The recombinant host cell of claim 1, wherein the fatty acid derivative biosynthetic polypeptide has ester synthase activity and the fatty acid derivative composition comprises a fatty ester.

8. The recombinant host cell of claim 2, wherein said higher titer of the recombinant host cell is from at least about 10% to at least about 90% greater compared to the corresponding wild type host cell.

9. The recombinant host cell of claim 2, wherein said higher yield of the recombinant host cell is from at least about 5% to at least about 80% greater compared to the corresponding wild type host cell.

10. The recombinant host cell of claim 2, wherein the fatty acid derivative composition is produced at a titer of from about 100 mg/L to about 300 g/L.

11. The recombinant host cell of claim 10, wherein the fatty acid derivative composition is produced at a titer of from about 1 g/L to about 250 g/L.

12. The recombinant host cell of claim 10, wherein the fatty acid derivative composition is produced at a titer of at least about 30 g/L.

13. The recombinant host cell of claim 2, wherein the fatty acid derivative composition is produced at a productivity of from about 0.7 mg/L/hr to about 2.5 g/L/hr.

14. The recombinant host cell of claim 1, wherein the ACP is a cyanobacterial acyl carrier protein (cACP).

15. The recombinant host cell of claim 1, wherein the ACP is a Marinobacter aquaeolei VT8 acyl carrier protein (mACP).

16. The recombinant host cell of claim 1, wherein the ACP is an E. coli acyl carrier protein (ecACP).

17. The recombinant host cell of claim 1, further comprising an sfp gene encoding a 4′-phosphopantetheinyl transferase protein.

18. The recombinant host cell of claim 17, wherein the sfp gene is a B. subtilis sfp gene.

19. The recombinant host cell of claim 1, wherein the fatty acid derivative composition is produced extracellularly or intercellularly.

20. A cell culture comprising the recombinant host cell of claim 1.

21. The cell culture of claim 20, wherein the fatty acid derivative composition is found in a culture medium.

22. The cell culture of claim 21, wherein the fatty acid derivative composition comprises at least one fatty acid derivative selected from the group consisting of a fatty acid, a fatty alcohol and a fatty ester.

23. The cell culture of claim 22, wherein the fatty acid derivative is a C₆, C₈, C₁₀, C₁₂, C₁₃, C₁₄, C₁₅, C₁₆, C₁₇, or C₁₈ fatty acid derivative.

24. The cell culture of claim 23, wherein the fatty acid derivative is a C_{10:1}, C_{12:1}, C_{14:1}, C_{16:1}, or C_{18:1} unsaturated fatty acid derivative.

25. The cell culture of claim 22, wherein the fatty acid derivative composition comprises a fatty acid.

26. The cell culture of claim 22, wherein the fatty acid derivative composition comprises a fatty alcohol.

27. The cell culture of claim 22, wherein the fatty acid derivative composition comprises a fatty ester.

28. The cell culture of claim 22, wherein the fatty acid derivative composition comprises a fatty acid derivative having a double bond between the 7th and 8th carbon from the reduced end of the fatty acid, the fatty ester, or the fatty alcohol.

29. The cell culture of claim 22, wherein the fatty acid derivative composition comprises an unsaturated fatty acid derivative.

30. The cell culture of claim 22, wherein the fatty acid derivative composition comprises a saturated fatty acid derivative.

31. The cell culture of claim 22, wherein the fatty acid derivative composition comprises a branched chain fatty acid derivative.

32. The cell culture of claim 22, wherein the fatty acid derivative has a fraction of modern carbon of about 1.003 to about 1.5.

33. The cell culture of claim 22, wherein the fatty acid derivative has a δ¹³C of from about −10.9 to about −15.4.

34. A method of making a fatty acid derivative composition, comprising the steps of:
(a) culturing the recombinant host cell of claim 1 in the presence of a carbon source in order to produce a fatty acid derivative composition; and
(b) collecting the fatty acid derivative composition from the culture medium.

35. The method of claim 34, wherein a yield, titer or productivity of the fatty acid derivative composition is at least about 10% greater than the yield, titer or productivity of a fatty acid derivative composition produced by a corresponding wild type host cell cultured under the same conditions.

36. The method of claim 34, further comprising optionally isolating the fatty acid derivative composition from the recombinant host cell.

37. The method of claim 34, wherein the fatty acid derivative composition is selected from the group consisting of a fatty acid, a fatty alcohol, a fatty ester, a fatty aldehyde, an alkane, an alkene, an olefin, and a ketone.

38. The method of claim 37, wherein the fatty acid derivative composition is a combination of any one or more fatty acid derivatives.

39. The method of claim 34, wherein the fatty acid derivative biosynthetic protein expressed in the recombinant host cell has thioesterase activity and the fatty acid derivative composition comprises a fatty acid.

40. The method of claim 34, wherein the recombinant host cell is further engineered to express a protein with carboxylic acid reductase (CAR) activity and the fatty acid derivative composition comprises a fatty alcohol.

41. The method of claim 34, wherein the fatty acid derivative biosynthetic protein expressed in the recombinant host cell has acyl ACP reductase (AAR) activity and the fatty acid derivative composition comprises a fatty alcohol.

42. The method of claim 34, wherein the fatty acid derivative biosynthetic protein expressed in the recombinant host cell has ester synthase activity and the fatty acid derivative composition comprises a fatty ester.

43. The method of claim 34, wherein the ACP is a cyanobacterial acyl carrier protein (cACP).

44. The method of claim 43, wherein the cACP is a Marinobacter aquaeolei VT8 acyl carrier protein (mACP).

45. The method of claim 34, wherein the ACP is an E. coli acyl carrier protein (ecACP).

46. The method of claim 34, wherein the phosphopantetheinyltransferase protein is a 4′-phosphopantetheinyl transferase protein encoded by an sfp gene.

47. The method of claim 46, wherein the sfp gene is a B. subtilis sfp gene.

48. The method of claim 34, wherein the fatty acid derivative composition is found in the culture medium.

49. The method of claim 34, wherein the fatty acid derivative composition comprises a C₆, C₈, C₁₀, C₁₂, C₁₃, C₁₄, C₁₅, C₁₆, C₁₇, or C₁₈ fatty acid derivative.

50. The method of claim 49, wherein the fatty acid derivative composition comprises a C_{10:1}, C_{12:1}, C_{14:1}, C_{16:1}, or C_{18:1} unsaturated fatty acid derivative.

51. The method of claim 34, wherein the fatty acid derivative composition comprises a fatty acid.

52. The method of claim 34, wherein the fatty acid derivative composition comprises a fatty alcohol.

53. The method of claim 34, wherein the fatty acid derivative composition comprises a fatty ester.

54. The method of claim 34, wherein the fatty acid derivative composition comprises a fatty acid derivative having a double bond between the 7th and 8th carbon from the reduced end of the fatty acid, the fatty ester, or the fatty alcohol.

55. The method of claim 34, wherein the fatty acid derivative composition comprises an unsaturated fatty acid derivative.

56. The method of claim 34, wherein the fatty acid derivative composition comprises a saturated fatty acid derivative.

57. The method of claim 34, wherein the fatty acid derivative composition comprises branched chain fatty acid derivative.

58. The method of claim 34, wherein the fatty acid derivative has a fraction of modern carbon of about 1.003 to about 1.5.

59. The method of claim 34, wherein the fatty acid derivative has a δ¹³C of from about −10.9 to about −15.4.
